(19) United States
(12) Patent Application Publication
(10) Pub. No.: US 2016/0122825 A1
(43) Pub. Date: May 5, 2016

(54) EFFICIENT FUNCTIONAL GENOMICS PLATFORM

(71) Applicant: Board of Regents, The University of Texas System, Austin, TX (US)

(72) Inventors: Gordon B. MILLS, Houston, TX (US); Yiling LU, Houston, TX (US); Han LIANG, Houston, TX (US); Wai Ting CHEUNG, Houston, TX (US)

(73) Assignee: Board of Regents, The University of Texas System, Austin, TX (US)

(21) Appl. No.: 14/409,726

(22) PCT Filed: Jun. 26, 2013
(86) PCT No.: PCT/US2013/047982

§ 371 Date: Dec. 19, 2014

Related U.S. Application Data
(60) Provisional application No. 61/664,497, filed on Jun. 26, 2012.

(51) Int. Cl. C12Q 1/68; A61K 38/17

(52) U.S. Cl. CPC C12Q 1/6886; A61K 38/1709; C12Q 2600/158; C12Q 2600/136; C12Q 2600/156

(57) ABSTRACT

Methods for identifying oncogenic biomarkers specific to a patient's cancer. Methods are also provided for identifying candidate therapeutic agents for treating a patient's cancer based on the oncogenic biomarkers. Methods for treating patient's having cancers that express mutant PIK3R1 and ras genes are also disclosed.

The present application claims the priority benefit of U.S. provisional application No. 61/664,497, filed Jun. 26, 2012, the entire contents of which are incorporated herein by reference.

### BACKGROUND OF THE INVENTION

1. Field of the Invention

The present invention relates generally to the field of molecular biology and oncology. More particularly, it concerns cancer genetics and cancer cell specific diagnostics and therapeutics. In some aspects, disclosed methods are most optimally applied to provide individualized patient therapy (also known as personalized cancer therapy or stratified patient therapy).

2. Description of Related Art

Recent advances in next-generation sequencing technology have enabled the unprecedented characterization of a full spectrum of somatic alterations in cancer genomes (Mardis 2011). In particular, through target-enrichment, whole-exome sequencing represents a cost-effective strategy to identify mutations in protein-coding exons in the human genome. Further with continued decreases in costs, whole genome sequencing and RNASeq is providing additional information on copy number, rearrangements and alternate splicing. In the past several years, this approach has been successfully applied to several human cancers lineages (TCGA 2008; Jones et al. 2010; Gui et al. 2011; TCGA 2011; Wang et al. 2011). Given the large numbers of somatic mutations typically detected by these approaches, a key challenge and to date unresolved problem in the downstream analysis is to distinguish “drivers” that functionally contribute to tumorigenesis from “passengers” that occur as the consequence of genomic instability.

### SUMMARY OF THE INVENTION

In a first embodiment there is provided a method of identifying an oncogenic biomarker in a patient having a cancer, comprising (a) obtaining genomic sequences (e.g., genomic DNA sequences or expressed RNA sequences) of a patient's cancer; (b) identifying a plurality of genes that are mutated in the patient's cancer; (c) analyzing the plurality of mutant genes to determine the presence of one or more genes having an oncogenic biomarker. In some aspects, analyzing the plurality of mutant genes comprises (i) expressing the plurality of genes, or inhibitory nucleic acids targeted to the genes, in cells; and (ii) analyzing the effect the expression in cells to determine the presence of one or more genes having an oncogenic biomarker.

In a further embodiment there is provided a method of selecting a drug to treat a patient having a cancer, comprising (a) obtaining genomic sequences (e.g., genomic DNA sequences or expressed RNA sequences) of a patient's cancer; (b) identifying a plurality of genes that are mutated in the patient's cancer; (c) analyzing the plurality of mutant genes to determine the presence of one or more genes having an oncogenic biomarker; and (d) selecting one or more candidate agents to treat the patient on the basis of said analysis. Accordingly, in some aspects, analyzing the plurality of mutant genes comprises (i) expressing the plurality of genes, or inhibitory nucleic acids targeted to the plurality of genes, in cells; and (ii) analyzing the effect the expression in the cells to determine the presence of one or more genes having an oncogenic biomarker. In further aspects, selecting one or more candidate agents comprises (iv) screening for an agent effective against cells that express a gene having an oncogenic biomarker, or that express an inhibitory nucleic acid targeted to a gene having an oncogenic biomarker. In some aspects, a method of selecting a drug in accordance with the embodiments is carried out in about or not more than about 1, 2, 3, 4, 5 or 6 months.

In yet a further embodiment there is provided a method for identifying an oncogenic biomarker in a patient having a cancer, the method comprising: (a) obtaining expressed RNA sequences of the patient's cancer; (b) identifying from said sequences a plurality of genes that are mutated (e.g., have undergone RNA editing) in the patient's cancer; and (c) analyzing the plurality of mutant genes to determine the presence of one or more genes having an oncogenic biomarker. In a related embodiment a method is provided for selecting a drug to treat a patient having a cancer, the method comprising: (a) obtaining expressed RNA sequences of the patient's cancer; (b) identifying from said sequences a plurality of genes that are mutated (e.g., have undergone RNA editing) in the patient's cancer; (c) analyzing the plurality of mutant genes to determine the presence of one or more genes having an oncogenic biomarker; and (d) selecting one or more candidate agents to treat the patient on the basis of said analysis. Thus, in some aspects, the plurality of genes comprise genes encoding RNAs that are edited in cancer cells (resulting in one or more C->U or A->I mutations). Accordingly, identifying from said sequence a plurality of genes that are mutated in the patient's cancer can comprise comparing the expressed RNA sequences to a reference sequence or to a genomic DNA sequence from the patient to identify positions of RNA editing (or an elevated level of RNA editing). In certain aspects, multiple reads of the expressed RNA sequences are obtained and, in some aspects, identifying a plurality of genes that are mutated in the patient's cancer further comprises quantifying the proportion of C->U or A->I mutations in RNA sequences. Thus, in certain aspects, a method of the embodiments comprises one or more of the steps outlined in FIG. 17.

In a further embodiment there is provided a method of selecting a target for treatment of a patient having a cancer (or for development of a targeted therapeutic), comprising (a) obtaining genomic sequences of a patient's cancer; (b) identifying a plurality of genes that are mutated in the patient's cancer; (c) analyzing the plurality of mutant genes to determine the presence of one or more genes having an oncogenic biomarker; and (d) selecting one or more candidate agents to treat the patient on the basis of said analysis. Accordingly, in some aspects, analyzing the plurality of mutant genes comprises (i) expressing the plurality of genes, or inhibitory nucleic acids targeted to the plurality of genes, in cells; and (ii) analyzing the effect the expression in the cells to determine the presence of one or more genes having an oncogenic biomarker. In further aspects, selecting one or more candidate targets comprises (iv) screening for an agent effective against cells that express a gene having an oncogenic biomarker, or that express an inhibitory nucleic acid targeted to a gene having an oncogenic biomarker. A target is any gene or protein in a pathway targeted by any agent effective against cells that express a gene having an oncogenic biomarker, or that express an inhibitory nucleic acid targeted to a gene having an oncogenic biomarker. In some aspects, a method of selecting a target in accordance with the embodiments is carried out in about or not more than about 1, 2, 3, 4, 5 or 6 months.

In certain aspects of the embodiments, selecting one or more candidate agents comprises (iv) determining changes in signaling pathway activation in cells that express a gene having an oncogenic biomarker, or that express an inhibitory nucleic acid targeted to a gene having an oncogenic biomarker; and (v) selecting one or more candidate agents to treat the patient based on the changes in signaling pathway activation. For example, determining changes in signaling pathway activation can comprise performing test using an RNA expression array, quantitative RNA sequencing (e.g., RNA-Seq), reverse-phase protein array or other method for measuring RNA or protein levels or function. In some cases, signaling pathway activation is determined by assessing a change in overall protein or RNA expression. In still further aspects, signaling pathway activation is determined by assessing a change in protein phosphorylation, methylation, acetylation, glycosylation or localization. For example, in some aspects, cells comprising an oncogenic biomarker will exhibit increased activity in a specific signaling pathway (e.g., JNK, MEK or p38MAPK) and a drug or prodrug that targets various components of the upregulated pathway is selected.

In yet further aspects, selecting one or more candidate agents according to the embodiments comprises (iv) screening for an agent effective against cells that express a gene having an oncogenic biomarker, or express an inhibitory nucleic acid targeted to a gene having an oncogenic biomarker. For example, an effective agent can be an agent that decrease cell proliferation, increases cell death, decreases motility or invasion, increases anchorage dependence, increases apoptosis, changes gene expression (e.g., changes expression of a reporter gene), changes RNA or protein expression, or changes growth factor dependence in the expressing cells. Agents for use in screening in accordance with the embodiments include, without limitation, small molecules (e.g., kinase inhibitors), antibodies, inhibitory nucleic acids, polypeptides or hormones.

In still further aspects, selecting one or more candidate agents in accordance with the embodiments further comprises reporting the identity of the selected agent(s). For example, the reporting can comprise providing an oral, written or electronic report. In some cases, the report is provided to the patient. In further cases the report is provided to a health care worker (e.g., a patient's doctor), a hospital or an insurance company.

In a further aspect, a method of the embodiments can further comprise administering the candidate agent or a prodrug thereof to the patient. Thus, in some aspects, there is provided a method of treating a patient having a cancer comprising (a) obtaining the results of an analysis in accordance with embodiments and (b) causing the patient to be treated with the one or more agents (or prodrugs thereof) selected by the analysis. In some aspects, the one or more agents is administered to the patient two, three, four, five or more times. The method for administering a selected agent will vary depending upon the specific agent selected, but can include without limitation, administration intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, via inhalation (e.g. aerosol inhalation), by injection or by infusion.

In still further aspects, a selected agent(s) is administered in conjunction with at least a second anti-cancer therapy. For instance, the selected agent(s) can be administered before, after or essentially simultaneously with said second therapy. Examples of a second anticancer therapy include, without limitation a surgical, radiation, hormonal, cancer cell-targeted or chemotherapeutic anticancer therapy.

In some aspects, identifying a plurality of genes that are mutated in the patient's cancer (step (b)) further comprises identifying genes based on an algorithm. For example, the algorithm can be a computational algorithm that predicts mutations that are most likely to contribute to oncogenesis and therefore most likely to serve as oncogenic biomarkers.

Certain aspects of the embodiments refer to genes that are mutated in the patient's cancer. As use herein a “mutant” gene refers to a gene or an expressed RNA that comprises at least a first altered nucleotide position in a cell of interest (such as a cancer cell) relative to a control cell. Such alterations may include deletions, insertions, inversions, rearrangements, nucleic acid substitutions, and/or epigenetic modifications (e.g., alteration in DNA methylation or hydroxymethylation) in an RNA coding region or in gene expression control elements such promoters or enhancers. In the case of an expressed RNA, a mutation may also encompass a position that undergoes aberrant RNA editing or alternative splicing relative to RNA in a control cell. Thus, mutated genes can refer to genes that comprise a mutation in the coding as well as non-coding sequences. Likewise, a gene that is dysregulated, for example, by amplification, complete deletion (e.g., loss of heterozygosity) or by genetic changes in the promoter or enhancer sequences or that is that is rearranged are included in the term mutant gene. In some aspects, mutant genes can encode polypeptides or functional RNAs (e.g., miRNAs). In certain cases a mutant gene is identified by comparison with a gene from a non-cancer cell (e.g., from the same patient) or with a reference genome.

Certain aspects of the embodiments concern determining the presence of one or more genes having an oncogenic biomarker by analysis of the effect of expression of the gene, or of inhibitory nucleic acid targeted to the gene, in a cell. For example, such analysis can comprises determining a change in gene expression, cell proliferation, anchorage dependent growth, apoptosis or growth factor dependence upon expression of the gene, overexpression of the gene or expression of an inhibitory nucleic acid targeted to the gene. In some cases, determining the presence of a gene comprising an oncogenic biomarker can comprise use of cells in vitro or in vivo (e.g., cells growing in a lab animal, such as a mouse). The effect of expression of the gene, or of inhibitory nucleic acid targeted to the gene, can be assessed in a cell in culture and then introduced into a mouse or by direct introduction of the gene, or of inhibitory nucleic acid targeted to the gene into a mouse.

Cells for use according to embodiments include, without limitation, primary cells or tissue culture cells. In some aspects, the cells are cancer cells that are from the same type of cancer as the patient's cancer. For example, in some aspects the cells are epithelial cancer cells or endometrial cancer cells (e.g., EFE184, SK-UT2, SNG-II or KLE cells). In some aspects, the cells are mammalian cells such as human, nonhuman primate or murine cells. In still further aspects, the cells are growth factor dependent cells, such as cells that require one or more exogenous growth factors (e.g., cells that require a growth factor unless transformed with an oncogene). In some specific examples, the cells are IL-3 dependent myeloid cells, such as Ba/F3 cells or a derivative thereof. In some cases, the cells are EGF and/or adherence dependent (such as in MCF10A cells). In still further aspects, cells for use according to the embodiments can be modified to alter their ability to undergo apoptosis, autophagy or senescence (e.g., such as by altering p21 expression).

Accordingly, in some specific aspects, determining the presence of one or more genes having an oncogenic biomarker comprises identifying one or more genes that, upon expression of the gene, or an inhibitory nucleic acid to the gene, allow IL-3 dependent myeloid cells to proliferate in the absence of IL-3.

Accordingly, in some specific aspects, determining the presence of one or more genes having an oncogenic biomarker comprises identifying one or more genes that, upon expression of the gene, or an inhibitory nucleic acid to the gene, allow growth factor or anchorage dependent epithelial cells either derived from normal tissue or tumors to grow in the absence of the growth factor or in anchorage independent conditions.

Certain aspects of the embodiments concern obtaining genomic sequences of the patient's cancer. In some case, obtaining a sequence comprises obtaining a sequence of genomic DNA, expressed RNA or exon sequences. In further aspects, the genomic sequences can comprise a sequence of, about or at least about, 10×, 20×, 30×, 40×, 50×, 60×, 70×, 80× or more coverage of the cancer genome or exome. Thus, in some aspects, a method of the embodiments comprises quantifying the prevalence of a mutation in a set of sequences. In certain aspects, obtaining genomic sequences comprises obtaining both sequence of genomic DNA and expressed RNAs. Thus, in some aspects, a method of the embodiments can comprise comparing the genomic DNA and expressed RNA sequences (e.g., to identify positions that are subject to alternate splicing or RNA editing). In still further aspects, a method comprises comparing obtained sequences with genomic sequences of non-cancer cells (e.g., non-cancer cells in the patient).

In some aspects, obtaining the sequence in accordance with the embodiments comprises sequencing nucleic acid (i.e., DNA or RNA) in a biological sample from the patient. In some aspects the biological sample is cancer cell sample, such as a tumor biopsy or aspirate sample. In further aspects, the sample can be a tissue sample (e.g., a urine, serum or plasma sample) from the patient that comprises nucleic acid from cancer cells. Thus, in some aspects, a method of the embodiments comprises obtaining a biological sample from the patient.

In yet a further embodiment a method is provided for identifying a patient as having an oncogenic biomarker. For instance, such a method can comprise (a) determining whether a cancer or precancerous lesion in the patient comprises: (i) increased activity of, or mutation in, erbB3, PIK3R1, AMOTL2, CopA, ANKRD10, or RPS6KC1 relative to normal tissue; and/or (ii) decreased activity of, or mutation in, ARID1A, INHBA, KMO, TTLL5, GRM8, IGFBP3, AKTIP, PKHA2, TRPS1 or WNT11 relative to normal tissue, wherein increased activity of, or mutation in, erbB3, PIK3R1, CopA, AMOTL2, ANKRD10 or RPS6KC1 or decreased activity of, or mutation in, ARID1A, INHBA, KMO, TTLL5, GRM8, IGFBP3, AKTIP, PKHA2, TRPS1 or WNT11 indicates that the patient has an oncogenic biomarker. In yet a further embodiment, a method is provided for identifying a patient as having an oncogenic biomarker comprising (a) determining whether a cancer or precancerous lesion in the patient comprises (i) increased activity of, or mutation in, erbB3, PIK3R1, CopA, AMOTL2, ANKRD10 or RPS6KC1 relative to normal tissue; and/or (ii) decreased activity of, or mutation in, ARID1A, INHBA, KMO, TTLL5, GRM8, IGFBP3, AKTIP, PKHA2, TRPS1 or WNT11 relative to normal tissue; and (b) identifying the patient as having an oncogenic biomarker if the cancer or precancerous lesion comprises increased activity of, or mutation in, erbB3, PIK3R1, CopA, AMOTL2, ANKRD10 or RPS6KC1 or decreased activity of, or mutation in, ARID1A, INHBA, KMO, TTLL5, GRM8, IGFBP3, AKTIP, PKHA2, TRPS1 or WNT11; or identifying the patient as not having an oncogenic biomarker if the cancer or precancerous lesion in the patient does not comprise increased activity of, or mutation in, erbB3, PIK3R1, CopA, AMOTL2, ANKRD10 or RPS6KC1 or decreased activity of, or mutation in, ARID1A, INHBA, KMO, TTLL5, GRM8, IGFBP3, AKTIP, PKHA2, TRPS1 or WNT11.

Thus, certain aspects of the embodiments concern determining whether a patient's cancer has increased activity of, or mutation in (e.g., resulting from RNA editing or alternate splicing), erbB3, PIK3R1, AMOTL2, CopA, ANKRD10 or RPS6KC1 relative to normal tissue; and/or (ii) decreased activity of, or mutation in, ARID1A, INHBA, KMO, TTLL5, GRM8, IGFBP3, AKTIP, PKHA2, TRPS1 or WNT11. In some aspects, increased or decreased activity can be assessed by measuring protein or RNA expression from a gene. Accordingly, in some aspects, an increased or decreased activity can be an increased or decreased expression of a gene. In further aspects, a mutation is determined in a gene by obtaining all or part of the sequence of the gene in the patient's cancer. Mutations in the gene that can be determined in accordance with the embodiments include, without limitation, substitutions (at one or multiple nucleotides), deletions, insertions, inversions, amplifications or rearrangements. For example, in some aspects, the mutation is an amplification or deletion of an entire gene or the coding sequences thereof.

In still further aspects, a method of the embodiments further comprises administering an anti-cancer therapy to a patient identified as having an oncogenic biomarker. In some cases, a method comprises administering an aggressive anti-cancer therapy (e.g., a combination therapy or a therapy with greater potential side effects) to a patient identified as having an oncogenic biomarker. For instance, the anticancer therapy can include, without limitation, a surgical, radiation, hormonal, cancer cell-targeted or chemotherapeutic anticancer therapy.

Some aspects of the embodiments concern determining an increased activity of, or mutation in, PIK3R1 (e.g., to identify the presence of an oncogenic biomarker). For example, in some aspects, a mutation in PIK3R1 comprises introduction of a premature stop codon that results in a truncated protein coding sequence. For example, in some aspects, the mutation is the introduction of a stop codon between sequences encoding amino acid 50 and 450 (e.g., between amino acid positions 100 and 400, 150 and 400, 200 and 400, 250 and 400 or 300 and 400). Examples of specific PIK3R1 mutations that indicate the presence of an oncogenic biomarker include, without limitation, an E160*, R348*, R503W, R574fs (frame shift) or T576de1 mutation (positions indicated relative to the PIK3R1 protein coding sequence).

Further aspects of the embodiments concern determining a decreased activity of, or mutation in, AKTIP (e.g., to identify the presence of an oncogenic biomarker). For example, in some aspects, a reduced activity of AKTIP is determined by detecting a reduced expression of an AKTIP RNA or polypeptide. In further aspects, a mutation in AKTIP is determined by obtaining all or part of AKTIP gene sequence from a patient's cancer. For example, the mutation in AKTIP can be an inactivating deletion. Examples of specific AKTIP mutations that indicate the presence of an oncogenic biomarker include, without limitation, a Q281K mutation (indicated relative to the AKTIP protein coding sequence).

Certain aspects of the embodiments concern determining a decreased activity of, or mutation in, INHBA (e.g., to identify the presence of an oncogenic biomarker). For example, in some aspects, a reduced activity of INHBA is determined by detecting a reduced expression of an INHBA RNA or polypeptide. In further aspects, a mutation in INHBA is determined by obtaining all or part of INHBA gene sequence from a patient's cancer. For example, the mutation in INHBA can be an inactivating deletion. Examples of specific INHBA mutations that indicate the presence of an oncogenic biomarker include, without limitation, a R310Q mutation (indicated relative to the INHBA protein coding sequence).

In still a further embodiment a method for treating a cancer patient is provided, wherein it was determined that cancer cells in the patient comprise a mutation in a PIK3R1 that truncates the PIK3R1 open reading frame (ORF), the method comprising administering a MEK or JNK inhibitor therapy to the patient. For example, the mutation in a PIK3R1 can be a mutation that truncates the PIK3R1 ORF and results in a truncation between amino acid positions 50 and 450 of the PIK3R1 polypeptide (e.g., between amino acid positions 100 and 400, 150 and 400, 200 and 400, 250 and 400 or 300 and 400). In some specific aspects the PIK3R1 mutation results in a stop codon at position R348* of the PIK3R1 polypeptide.

In yet a further embodiment a method is provided for identifying a cancer patient having a biomarker for response to a MEK or JNK inhibitor therapy comprising (a) determining whether cancer cells in the patient comprise a mutation in a PIK3R1 gene that truncates the PIK3R1 ORF (e.g., between amino acid positions 50 and 450), wherein the presence of the mutation in PIK3R1 indicates the patient has a biomarker for response to a MEK or JNK inhibitor therapy. In a further aspect, a method of the embodiments further comprises (b) identifying the patient as having a biomarker for response to a MEK or JNK inhibitor therapy if the patient comprises a cancer with the mutation in PIK3R1; or identifying the patient as not having a biomarker for response to a MEK or JNK inhibitor therapy if the patient does not comprise a cancer with the mutation in PIK3R1.

In still yet a further embodiment a method for treating a cancer patient is provided, wherein it was determined the cancer cells in the patient comprise a KRAS oncogene, the method comprising administering a MEK or p38MAPK inhibitor therapy to the patient. Thus, in still a further embodiment, a method for identifying a cancer patient having a biomarker for response to a MEK or p38MAPK inhibitor therapy is provided comprising (a) determining whether cancer cells in the patient comprise a KRAS oncogene, wherein the presence of a KRAS oncogene indicates that the patient has a biomarker for response to a MEK or p38MAPK inhibitor therapy. In a further aspects, a method of the embodiments further comprises (b) identifying the patient as having a biomarker for response to a MEK or p38MAPK inhibitor therapy if the patient comprises a cancer with KRAS oncogene; or identifying the patient as not having a biomarker for response to a MEK or p38MAPK inhibitor therapy if the patient does not comprise a cancer with a KRAS oncogene.

Certain aspects of the embodiments concern a patient having a cancer. For example the patient can have an oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer. In some aspects, the patient has an epithelial cancer. In yet further aspects, the patient has an endometrial cancer, an ovarian cancer or a melanoma. In further aspects, the patient is a patient that has previously received one or more anti-cancer therapy or has pervious failed to adequately respond to one or more anti-cancer therapy. Thus, in some aspects, the cancer is a cancer that is resistant to at least a first anti-cancer therapy.

Certain aspects of the embodiments concerning administering a MEK or JNK inhibitor therapy to a patient, such as a patient having a mutation in a PIK3R1 gene. For example, the MEK or JNK inhibitor therapy can comprise administering a small molecule MEK or JNK inhibitor or a prodrug thereof. Examples of MEK inhibitors include, without limitation, PD0325901, PD98059, AZD6244 (Selumetinib), CI1040 (PD 184352), U0126-EtOH, AS703026, GSK1120212 (JTP-74057), TAK-733, AZD8330, PD318088, RDEA119 (BAY 869766), XL518 and/or GDC-0973. Non-limiting examples of JNK inhibitors include SP600125, JNK Inhibitor X, AEG3482, AS601245 (1,3-benzothiazol-2-yl(2-{[2-(3-pyridinyl)ethyl]amino}-4 pyrimidinyl) acetonitrile), Dicoumarol, and/or 5-Nitro-2-(3-phenylpropylamino)benzoic acid. In some aspects a MEK or JNK inhibitor therapy can be administered in conjunction with a second anti-cancer therapy such as any of the anticancer therapies detailed herein.

Still further aspects of the embodiments concern administering p38MAPK inhibitor therapy to a patient (e.g., a patient determined to have a cancer with KRAS oncogene). For example, the p38MAPK inhibitor therapy can comprise administering a small molecule p38MAPK inhibitor or a prodrug thereof. Examples of p38MAPK inhibitors include, without limitation, SB203580, SB202190 or AEG3482. In some aspects a p38MAPK inhibitor therapy can be administered in conjunction with a second anti-cancer therapy such as any of the anticancer therapies detailed herein.

In still further aspects, a method of the embodiments may be automated or performed by a computer. Thus, in a further embodiment there is provided a tangible computer-readable medium comprising a data base of driver mutations found in cancer cells. In some aspects, the database further comprises a corresponding candidate agent (for the driver mutations) predicted to be effective to inhibiting cancer cells that comprise the driver genetic mutation (or combination of driver mutations). For example, a database can comprising one or more (e.g., two, three, four or more) of the driver genetic mutations selected from: PIK3R1 E160*; PIK3R1 R348*; PIK3R1 R503W; PIK3R1 R574fs; PIK3R1 T576de1; AKTIP Q281K; AMOTL2 E507G; ANKRD10 D152G; and INHBA R310Q. In certain aspects, the database comprises at least a first driver genetic mutation that is a mutation resulting for an RNA editing event (e.g., rather than mutation of genomic DNA).

In a further embodiment there is provided a tangible computer-readable medium comprising computer-readable code that, when executed by a computer, causes the computer to perform operations comprising: (a) receiving information corresponding to a plurality of genetic mutations identified in a sample from a cancer patient; (b) comparing the a plurality of genetic mutations to a data base of driver mutations found in cancer cells; and (c) identifying a one or more driver mutations from the plurality of genetic mutations identified in the sample. In further aspects, the media further comprises computer-readable code that, when executed by a computer, causes the computer to perform operations comprising: (d) providing at least a first candidate agent predicted to be effective to inhibiting cancer cells in the patient based on the one or more identified driver mutations; or (d) calculating a ranked list of candidate agents predicted to be effective to inhibiting cancer cells in the patient based on the one or more identified driver mutations. In further aspects, receiving information comprises receiving from tangible data storage device information corresponding to a plurality of genetic mutations identified in a sample from a cancer patient. In still further aspects, a media comprises computer-readable code that, when executed by a computer, causes the computer to perform one or more additional operations comprising: sending information corresponding to one or more identified driver mutations and/or information corresponding to at least a first candidate agent predicted to be effective to inhibiting cancer cells in the patient to a tangible data storage device.

In certain aspects, a data base of driver mutations according to the embodiments comprises at least a first driver mutation that results from RNA editing of C->U or A->I in a RNA sequence. In further aspects, data base of driver mutations comprises one or more of the driver genetic mutations selected from: PIK3R1 E160*; PIK3R1 R348*; PIK3R1 R503W; PIK3R1 R574fs; PIK3R1 T576de1; AKTIP Q281K; AMOTL2 E507G; ANKRD10 D152G; and INHBA R310Q.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Overview of an example systems-biology approach to identifying cancer driver genes (e.g., in endometrial cancers).

FIG. 2: An example analytic pipeline for detecting somatic mutations in the exomes of endometrial tumors.

FIG. 3: Optimization of various parameters for tumor SNV calling. Additional filters were applied to boost tumor SNV calling accuracy; and the fraction of tumor SNV positions that are called as normal SNVs as an index for optimization.

FIG. 4: Mutation profile of somatic mutations in the exomes of endometrial cancer. Frequency of six classes of mutations is shown for all mutations in the exomes, non-silent coding mutations and silent mutations, respectively from left to right in each case.

FIG. 5: Novel candidate driver cancer genes identified by shRNA screening in Ba/F3 viability assay. (a) Ba/F3 cells were transfected with short-hairpin RNAs (shRNA) targeting indicated genes. Empty vector (pGIPZ) and non-specific shRNA served as the control. Cells were cultured without IL-3 for 4 weeks and harvested for viability assay. Cell viability relative to Ba/F3 parental cells was shown. * P<0.05, compared with Ba/F3 parental control. (b) Whole cell lysates were also collected for Western blotting with indicated antibodies, and ERK2 was used as the loading control.

FIG. 6: Western blots showing shRNA knock-down efficiency.

FIG. 7: Candidate driver cancer genes confirmed by overexpression of wild-type genes or mutants in Ba/F3 viability assay. Ba/F3 cells were transfected with wild-type (WT) or corresponding mutant(s) (mutation sites indicated) of (a) 6 positive genes in the shRNA screen and (b) 11 genes inactive in the screen. Cells transfected with shRNA were included in the assay as reference. pGIPZ vector is the empty vector carrying shRNA while LacZ corresponds to β-galactosidase in the pLenti6.3 vector. Cells were cultured without IL-3 for 4 weeks and harvested for viability assay. Cell viability relative to Ba/F3 parental cells was shown. * P<0.05, compared with Ba/F3 parental control. # indicates a significant difference in cell viability between WT- and mutant-transfected cells (P<0.05).

FIG. 8: Functional effect of candidate driver cancer genes by siRNA-mediated gene silencing in KLE endometrial cancer cell line. KLE cells were transfected with siRNAs targeting the indicated genes. Mock, risc-free siRNA and non-specific siRNA served as controls. (a) Efficacy of PTEN siRNA on AKT phosphorylation was determined by Western blotting. Cells transfected with indicated siRNAs were assayed for cell viability (b) 7 days or (c) 5 days post-transfection. Cell viability relative to mock transfected cells was shown. * P<0.05, compared with mock control.

FIG. 9a-c: Functional effect of candidate driver cancer genes by siRNA-mediated gene silencing in three additional endometrial cancer cell lines. Studies were performed as detailed in FIG. 8 using the EFE184 (a); SK-UT2 (b); or SNG-II (c) cells.

FIG. 10a-d: Mutational and functional analysis of ARID1A on the activation of PI3K pathway. (a) Co-mutation patterns of ARID1A and key genes related to the PI3K pathway. (Upper panel) Mutation diagram in the full set of endometrial tumor samples (n=222). Each column represents a tumor and each row corresponds to a single gene. (Lower panel) Mutation or co-mutation frequencies are expressed as a percentage of all the samples, and the co-mutation frequencies from random expectation are shown in parenthesis for comparison. Genes with statistically significant co-mutations are shown in dark gray, accompanied with Bonferroni-corrected P values. (b) The functional effect of ARID1A mutation on protein expression of the PI3K pathway. Each arrow represents a protein marker with significant differential expression between ARID1A wild-type and mutated samples: dark grey and light gray arrows are for phosphorylated and total proteins with P<0.05 (two-sided t-test, FDR<0.1), respectively; light grey arrows next to S6 represent phosphorylated proteins with marginal significance P<0.07 (FDR<0.13). Activated genes are shown in dark grey, and genes without available protein expression data are shown in light grey. (c) The functional effect of ARID1A mutation on the phosphorylation of AKT and p70S6K in tumor samples in which both PTEN and PIK3CA genes are wild-type, and also PTEN expression is retained (n=47). P-values were calculated based on two-sided t test. The boxes represents the distribution of individual values from the lower 25th percentile to upper 75 percentile; solid line in the middle, median values; lower and upper whisker, 5th and 95th percentiles; small circles, outlier data points. (d) Four endometrial cancer cell lines were transfected with 20 nM ARID1A siRNA or non-specific siRNA and harvested after 72 hours for western blotting with indicated antibodies. Numerical values below each lane of the immunoblots represent the quantification of the relative protein level by densitometry.

FIG. 11: A schematic representing the mutation distribution along the ARID1A gene.

FIG. 12: Upper panel represents a schematic map of the PIK3R1 polypeptide coding sequence. Identified mutations are mapped onto the schematic. Mutations identified as oncogenic biomarkers are indicated with a dark grey arrow. Lower panel is graph indicating the relative survival Ba/F3 cells expressing the indicated PIK3R1 mutants relative to cells expressing WT PIK3R1 (in the absence of IL3). PIK3R1 mutants providing a significant increase in cell survival are indicated with an asterisk and dark grey arrow. The light grey arrow indicates a known PIK3R1 SNP.

FIG. 13a-b: Figure shows a Western blots to assess ERK1/2, p38MAPK and JNK activation (phosphorylation) in cells expressing various PIK3R1 or KRAS mutants. Samples were from Ba/F3 cells (a) or SKUT2 endometrial cancer cells (b). PIK3R1 R348*, R274 and KRAS expressing cells show increased activation of the ERK pathway. PIK3R1 R348* expressing cells also show increased activation of the JNK pathway. Only KRAS expressing cells have elevated p38MAPK pathway activity.

FIG. 14: To determine potential therapeutic liability of cells having PIK3R1 or KRAS mutations, cells are cultured with and without IL3, as indicated, and treated with various MEK, JNK or p38MAPK inhibitors. As indicated in p85 wild type cells, and E160* p85, there is no difference in the presence or absence of IL3, indicating that the cells are not dependent on MAPK pathway for survival. In contrast, KRAS is dependent on MEK and p38 (but not JNK). R348* p85 is highly dependent on MEK and JNK indicating unexpected therapeutic liabilities of cancer expressing this mutant gene.

FIG. 15: Kaplan-Meier survival curves illustrating the correlation between RNA editing enzymes and patient survival in endometrial cancer. Left: ADAR. Right: APOBEC1. Top curves are normal samples; bottom curves are overexpression/amplification samples.

FIG. 16: Differential expression of RNA editing enzymes among endometrial tumor subtypes. Left: ADAR. Right: ADARB1.

FIG. 17: Computation pipeline for inferring RNA editing events.

FIG. 18: Analysis of representative RNA editing sites with a potential functional role. (a) Editing level by normal versus tumor sample group. (b) Kaplan-Meier survival curves for with editing and without editing. Top curve is without editing; bottom curve is with editing. (c) Editing level by endometrioid versus serous histological subtype. (d) Editing level by tumor stage.

FIG. 19: AMOTL2 edited variant E507G was expressed along with control constructs in two sensor cell lines Ba/F3 and MCF10A (as indicated). As shown in FIG. 19, AMOTL2 E507G (RNA edited variant) increased proliferation in both sensor cell lines, and effect that could be reversed by expression of AMOTL2-specific shRNA.

FIG. 20: The figure shows results of a typical screen of editing events in the Ba/F3 cells. The first two lanes (pDest GFP and pGIPZ shRNA) are controls. The remaining constructs are in groups of three with the normal wild type (WT) sequence, the RNA edited sequence (MUT) and a shRNA knockdown control.

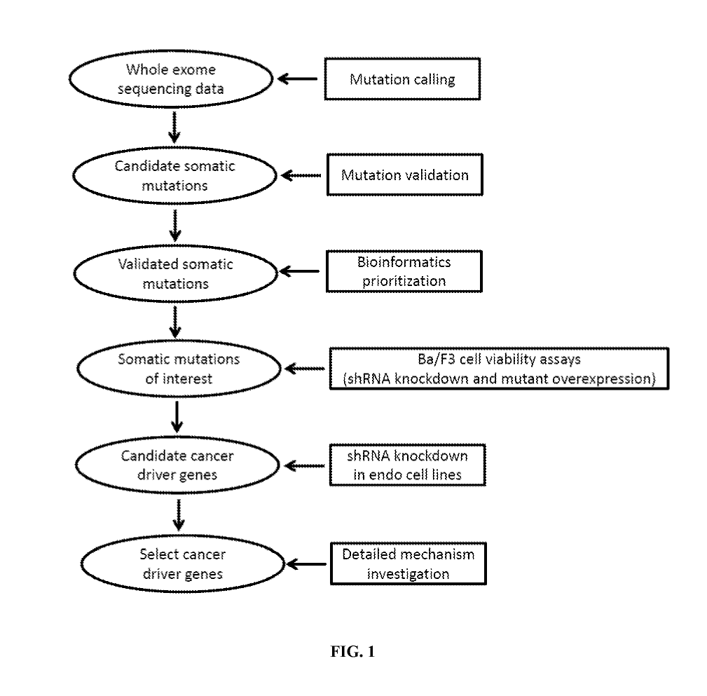

### DESCRIPTION OF ILLUSTRATIVE EMBODIMENTS

### I. The Present Invention

With the advent of cost effective next generation sequencing the amount of information that can be obtained concerning the particular genetic makeup of a cell has rapidly expanded. For example, a patient with a cancer could conceivably obtain a complete genetic sequence for their cancer along with, or shortly after, receiving a cancer diagnosis. Unfortunately, currently this information is of limited value because the possible effect of any observed genetic changes will be largely unknown and most genetic changes are meaningless. Thus, a central challenge to the field involves determining which aberrations in a given tumor represent “drivers” that determine tumor behavior (and can serve as oncogenic biomarkers) versus “passengers” resultant from the inherent instability of cancer genomes. Targeting driver aberrations should improve outcomes for patients, while targeting passenger aberrations would be without benefit and may instead result in unnecessary toxicity and delay implementation of effective therapies. It is critical to select the drug or drugs most likely to benefit each patient based on underlying aberrations in their own tumor. Unfortunately, previously there has been a complete lack of practical high-throughput platforms able to identify and select optimal therapies targeting driver aberrations.

In contrast, embodiments of the instant invention provide an efficient, high throughput, system to identify “driver” mutations that can serve as an oncogenic biomarker in individual cancers. First genomic sequences, such as expressed exon sequences (exome sequences) of the patient's cancer are obtained. From the sequence a plurality of mutations (relative to non-cancer cell sequence) are identified. In some cases the identified mutations can be computationally analyzed using an algorithm that identifies the most likely candidates as driver mutations. The effect of the mutations is then assessed by expression the mutant gene in cells. Alternatively or additionally, inhibitory RNA (e.g., siRNA) to the mutated gene is expressed in cells. The expressing cells are then observed for markers of a more or less transformed phenotype. The effect of such expression on the cells is then analyzed. For example, the growth characteristic, growth factor dependence, anchorage dependent growth or gene or protein expression can be observed in the cells. Based on these observations mutant genes that serve as oncogenes can be identified by observing a “more transformed” phenotype in cells that express the mutant gene. Conversely, tumor suppressor genes can be identified by observing a more transformed phenotype in cells expressing an inhibitory nucleic acid to the gene. Thus, using a rapid cell-based assay the oncogenic markers of an individual's particular cancer can be identified.

Once such oncogenic biomarkers have been identified in a cancer, methods are also provided for rapidly identifying effective therapeutics that target the particular oncogenic biomarker. For example, high throughput expression analysis (e.g., RNA or protein), such as studies that employ gene expression arrays or reverse-phase protein arrays, can be performed on cells that express an identified mutant gene (or an inhibitory nucleic acid to such a gene). These analyses can be used to identify one or more drugable pathway that is dysregulated in cells having a given biomarker and thereby provide a candidate drug for therapy (i.e., a drug that targets the dysregulated pathway). Alternatively or additionally, cells that express an identified mutant gene (or an inhibitory nucleic acid to such a gene) can be used to directly screen for agents that alter the phenotype of the expressing cells (e.g., agents that reduce proliferation, increase apoptosis or otherwise result in a “less transformed” phenotype in the transformed cells). Again, the result of the screen is to provide a candidate agent that can be used for individualized treatment of a cancer patient.

Importantly, because of the unique, high throughput cell based assays provided herein, a wide range of genetic alterations of each individual cancer can be assessed in parallel such that driver mutations can be determined in a time frame that can provide relevant guidance for therapy (e.g., with months). For example, using the current methodology, a patient having a cancer could have genes of the cancer sequenced and oncogenic biomarkers identified as they receive a first line cancer therapy. If, as all too often occurs, the patient fails to respond to the first line therapy then, by the time the patient is reassessed, an individualized therapeutic protocol based on the oncogenic biomarker harbored by the patient's cancer will be available. Such individualized therapy would be expected to be far more effective than merely guessing at a second or third line of anti-cancer therapy. Of equal importance the individualized therapy is far less likely to have severe side effects such unguided therapies. Thus, for the first time, the methods provided here offer the opportunity to identify the therapeutics that will be most effective for not only a specific individual, but for the specific cancer in the individual.

Using these new protocols, a range of oncogenic biomarkers is provided that can be used to assess cancer or precancerous lesions in a patient. In particular, the methods identified novel oncogenes and tumor suppressors that can serve as prognostic markers for the oncogenic transformation of cells. For example, increased activity of, or mutation (an activating mutation, e.g., from alternate splicing or RNA editing) in, erbB3, PIK3R1, AMOTL2, CopA, ANKRD10 or RPS6KC1 can serve as an oncogenic biomarker. Conversely, decreased activity of, or mutation (an inactivating mutation e.g., from alternate splicing or RNA editing) in, ARID1A, INHBA, KMO, TTLL5, GRM8, IGFBP3, AKTIP, PKHA2, TRPS1 or WNT11 is indicative of an oncogenic biomarker. For example, in the case of PIK3R1 mutation at E160*, R348*, R503W, R574fs or T576de1 is shown to serve as an oncogenic biomarker. Accordingly, the presence of such oncogenic biomarkers can be used to determine whether to provide and anticancer therapy to a patient and/or to determine how aggressive an anticancer therapy should be administered.

These new protocols have also succeeded in identifying new oncogenic biomarkers that can be used to determine which therapeutics cancer cells will respond to. Using these methods patient's are selected for a given cancer therapy based upon the oncogenic biomarker(s) in the patient's cancer. For example, a patient having a cancer that comprises a truncatation in the PIK3R1 gene open reading frame (e.g., R348*) can be selected for treatment with a JNK or MEK inhibitor. Similarly, a patient having a cancer comprising the KRAS oncogene can be selected for a MEK or p38MAPK inhibitor therapy.

### II. Detecting Oncogenic Biomarkers

Certain embodiments of the invention concern detecting an oncogenic biomarker in a patient's cancer. As used herein an oncogenic biomarker refers to a biomarker, such as genetic mutation (in genomic DNA or expressed RNA sequences) or alteration in gene expression in a cancer cell relative to a non-cancer cell that contributes to the transformed state of the cancer (i.e., a “driver” mutation). Thus, an oncogenic biomarker may be indicative the aggressiveness, metastatic potential or grade of the cancer. Likewise, and as demonstrated here oncogenic biomarkers can identify the agents that serve as candidate therapeutics for treatment of the cancer.

Thus, in some aspects, determining the presence of an oncogenic biomarker can comprise detecting changes in nucleic acid content (e.g., mutated nucleic acid sequence, such as by alternate splicing or RNA editing) or expression level in a cancer. In further aspects, determining the presence of an oncogenic biomarker can comprise detecting changes in polypeptide sequence, expression level or activity (e.g., detecting active or phosphorylated polypeptides).

A. Nucleic Acid Detection

Certain aspects of the embodiments concern obtaining as nucleic acid sequence of a cancer cell genome or a portion thereof by analysis of RNA or DNA. Methods for sequencing and quantifying amounts of DNA as well as cDNA are well known in the art and are commercially available. DNA sequencing, including single molecule sequencing, such as pyrosequencing or sequencing by ligation (e.g., SOLiD™), may be used to detect the presence, absence, or amount mutant gene. Such next generation sequencing methods may be particularly effective for high-throughput screening, for examining large regions of genomic DNA or for providing deep sequencing of a cancer cell genome or exome. In some embodiments, allele-specific primers may be used that incorporate single-nucleotide polymorphisms into the sequence of the sequencing primer (Wong et al., 2006).

Likewise, in some embodiments, assessing expression of an oncogenic biomarker, can involve quantifying mRNA expression. Northern blotting techniques are well known to those of skill in the art. Northern blotting involves the use of RNA as a target. Briefly, a probe is used to target an RNA species that has been immobilized on a suitable matrix, often a filter of nitrocellulose. The different species should be spatially separated to facilitate analysis. This often is accomplished by gel electrophoresis of nucleic acid species followed by “blotting” on to the filter. Subsequently, the blotted target is incubated with a probe (such as a labeled probe) under conditions that promote denaturation and rehybridization. Because the probe is designed to base pair with the target, the probe will bind a portion of the target sequence under renaturing conditions. Unbound probe is then removed, and detection is accomplished.

In some embodiments, nucleic acids are quantified following gel separation and staining with ethidium bromide and visualization under UV light. In some embodiments, if the nucleic acid results from a synthesis or amplification using integral radio- or fluorometrically-labeled nucleotides, the products can then be exposed to x-ray film or visualized under the appropriate stimulating spectra, following separation.

In some embodiments, visualization is achieved indirectly. Following separation of nucleic acids, a labeled nucleic acid is brought into contact with the target sequence. The probe is conjugated to a chromophore or a radiolabel. In another embodiment, the probe is conjugated to a binding partner, such as an antibody or biotin, and the other member of the binding pair carries a detectable moiety. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present embodiments.

In some embodiments, reverse transcription (RT) of RNA to cDNA followed by relative quantitative PCR™ (RT-PCR™) can be used to determine the relative concentrations of specific gene or gene product as well as mutations, rearrangement, insertions and deletions. By determining that the concentration of a specific mRNA or species of mRNA varies, it is shown that the gene encoding the specific mRNA species is differentially expressed. In certain aspects mRNA expression can be quantified relative to the expression of a control mRNA.

In some embodiments, the amplification products described above may be subjected to sequence analysis to identify specific kinds of variations or amounts of RNA or variants using standard sequence analysis techniques. Within certain methods, exhaustive analysis of genes is carried out by sequence analysis using primer sets designed for optimal sequencing. The present embodiments provide methods by which any or all of these types of analyses may be used. Using the sequences disclosed herein, oligonucleotide primers may be designed to permit the amplification of sequences throughout a cancer cell genome that may then be analyzed by direct sequencing. As discussed above, methods for such sequencing include, but are not limited to, reversible terminator methods (e.g., used by Illumina® and Helicos® BioSciences), pyrosequencing (e.g., 454 sequencing from Roche) and sequencing by ligation (e.g., Life Technologies™ SOLiD™ sequencing)

B. Protein Biomarker Detection

In some aspects, methods of the embodiments concern detection of the expression or activity of oncogenic biomarkers, by analysis of polypeptide expression. For example, immunodetection methods for binding, purifying, removing, quantifying and/or otherwise generally detecting protein components can be employed. Antibodies prepared in accordance with the present embodiments may be employed to detect biomarker expression and/or biomarker activation. Some immunodetection methods include enzyme linked immunosorbent assay (ELISA), radioimmunoassay (RIA), immunoradiometric assay, fluoroimmunoassay, chemiluminescent assay, bioluminescent assay, reverse and forward phase protein arrays, mass spectroscopy and Western blot to mention a few. The steps of various useful immunodetection methods have been described in the scientific literature, such as, e.g., Doolittle M H and Ben-Zeev O, 1999; Gulbis B and Galand P, 1993; De Jager R et al., 1993; and Nakamura et al., 1987, each incorporated herein by reference.

In general, the immunobinding methods include obtaining a sample suspected of containing a biomarker protein, polypeptide and/or peptide, and contacting the sample with a first anti-biomarker antibody in accordance with the present embodiments, under conditions effective to allow the formation of immunocomplexes.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

### III. Anti-Cancer Therapies

Certain aspects of the embodiments concern administering an anticancer therapy to a patient. In some aspects, anticancer therapies are identified by the methods detailed herein, such as therapies that target specific metabolic or signaling pathways. For example, the anticancer therapy can be a therapy that targets the JNK kinase, MEK kinase or p38MAPK pathway.

Compounds of the present embodiments may also exist in prodrug form. Since prodrugs are known to enhance numerous desirable qualities of pharmaceuticals (e.g., solubility, bioavailability, manufacturing, etc.), the compounds employed in some methods of the invention may, if desired, be delivered in prodrug form. In general, such prodrugs will be functional derivatives of the metabolic pathway inhibitors of the embodiments, which are readily convertible in vivo into the active inhibitor. Conventional procedures for the selection and preparation of suitable prodrug derivatives are described, for example, in “Design of Prodrugs”, ed. H. Bundgaard, Elsevier, 1985; Huttunen et al., 2011; and Hsieh et al., 2009, each of which is incorporated herein by reference in its entirety.

A prodrug may be a pharmacologically inactive derivative of a biologically active inhibitor (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulphate ester, or reduction or oxidation of a susceptible functionality. Thus, prodrugs of the compounds employed in the embodiments may be prepared by modifying functional groups present in the compound in such a way that the modifications are cleaved, either in routine manipulation or in vivo, to the parent compound. Prodrugs include, for example, compounds described herein in which a hydroxy, amino, or carboxy group is bonded to any group that, when the prodrug is administered to a subject, cleaves to form a hydroxy, amino, or carboxylic acid, respectively. Thus, the invention contemplates prodrugs of compounds of the present invention as well as methods of delivering prodrugs.

In order to increase the effectiveness of a therapy of the embodiments (e.g., a MEK, JNK or p38MAPK inhibitor therapy), it may be desirable to combine these therapies with other agents effective in the treatment of cancer. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This may be achieved by contacting the cell (or administering a subject) with a single composition or pharmacological formulation that includes both agents, or by contacting the cell with two distinct compositions or formulations, at the same time, wherein one composition includes, e.g., a kinase inhibitor inhibitor and the other includes the second agent(s).

Treatment with a therapy or agent of the embodiments may precede or follow the other agent or treatment by intervals ranging from minutes to weeks. In embodiments where the agents are applied separately to the cell, one would generally ensure that a significant period of time did not expire between the time of each delivery, such that the agents would still be able to exert an advantageously combined effect on the cell. In such instances, it is contemplated that one may contact the cell with both modalities within about 12-24 hours of each other and, more preferably, within about 6-12 hours of each other. In some situations, it may be desirable to extend the time period for treatment significantly where several days (e.g., 2, 3, 4, 5, 6 or 7 days) to several weeks (e.g., 1, 2, 3, 4, 5, 6, 7 or 8 weeks) lapse between the respective administrations.

Various combinations may be employed, where the MEK, JNK or p38MAPK inhibitor therapy is “A” and the secondary agent or treatment, such as radiotherapy, chemotherapy or targeted therapeutic, is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

In certain embodiments, administration of an agent of the present embodiments to a patient will follow general protocols for the administration of chemotherapeutics, taking into account the toxicity, if any, of the agent. It is expected that the treatment cycles would be repeated as necessary. It also is contemplated that various standard therapies, as well as surgical intervention, may be applied in combination with the described hyperproliferative cell therapy.

A. Chemotherapy

In some aspects, an anticancer therapy comprises administration of a chemotherapeutic agent. Likewise, combination chemotherapies may be used including, for example, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; carboplatin, procarbazine, plicomycin, gemcitabien, navelbine, farnesyl-protein tansferase inhibitors, transplatinum, and pharmaceutically acceptable salts, acids or derivatives of any of the above. In certain embodiments, the compositions provided herein may be used in combination with gefitinib. In certain embodiments, one or more chemotherapeutic may be used in combination with the compositions provided herein.

B. Radiotherapy

Other factors effective for cancer therapy and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic composition and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with an agent or therapy of the present embodiments. The general approach for combined therapy is discussed below. Generally, the tumor cell must bear some marker that is amenable to targeting, i.e., is not present on the majority of other cells. Many tumor markers exist and any of these may be suitable for targeting in the context of the present embodiments. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Gene Therapy

In yet another embodiment, the secondary treatment is a gene therapy in which a therapeutic polynucleotide is administered before, after, or at the same time as the therapeutic composition. Viral vectors for the expression of a gene product are well known in the art, and include such eukaryotic expression systems as adenoviruses, adeno-associated viruses, retroviruses, herpesviruses, lentiviruses, poxviruses including vaccinia viruses, and papiloma viruses, including SV40. Alternatively, the administration of expression constructs can be accomplished with lipid-based vectors such as liposomes or DOTAP:cholesterol vesicles. All of these method are well known in the art (see, e.g. Sambrook et al., 1989; Ausubel et al., 1998; Ausubel, 1996).

Delivery of a vector encoding one of the following gene products will have a combined anti-hyperproliferative effect on target tissues. A variety of proteins are encompassed within the present embodiments, some of which are described below.

As noted above, the tumor suppressor oncogenes function to inhibit excessive cellular proliferation. The inactivation of these genes destroys their inhibitory activity, resulting in unregulated proliferation.

Genes that may be employed as secondary treatment in accordance with the present embodiments include p53, p16, Rb, APC, DCC, NF-1, NF-2, FUS1, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors), MCC and other genes listed in Table IV.

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatments provided herein, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present embodiments may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other Agents

It is contemplated that other agents may be used in combination with the compositions provided herein to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-lbeta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the compositions provided herein by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the compositions provided herein to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the compositions provided herein to improve the treatment efficacy.

In certain embodiments, hormonal therapy may also be used in conjunction with the present embodiments or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Landscape of Somatic Mutations in the Exomes of Endometrial Cancer

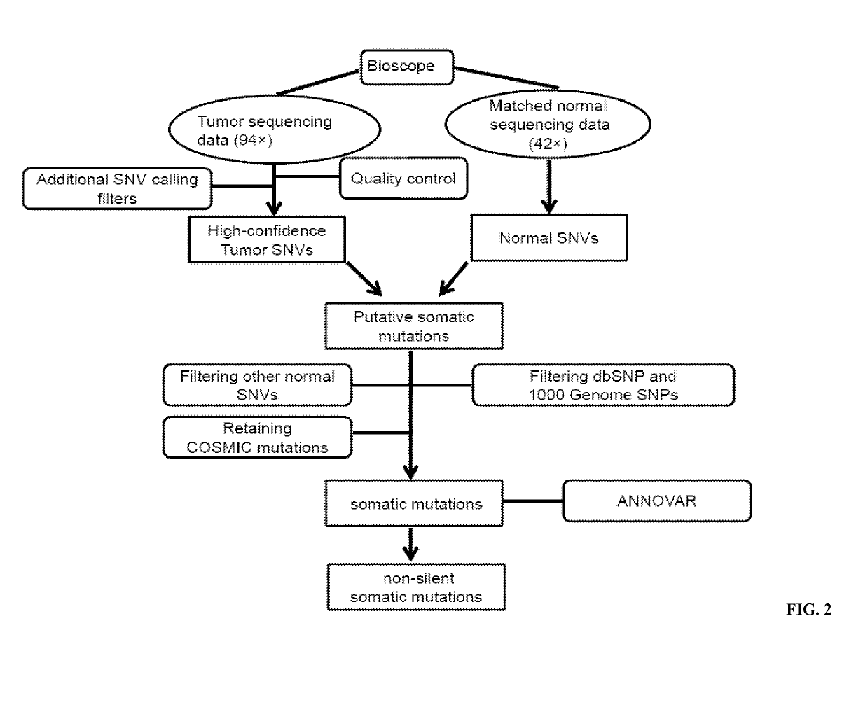

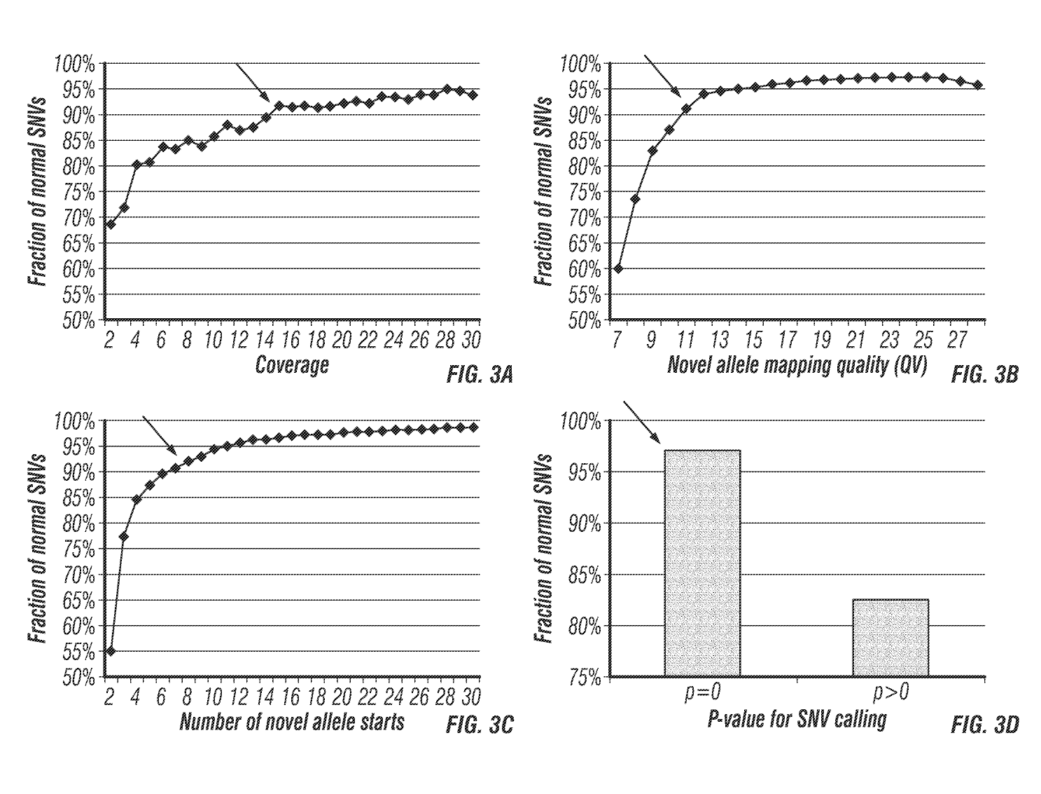

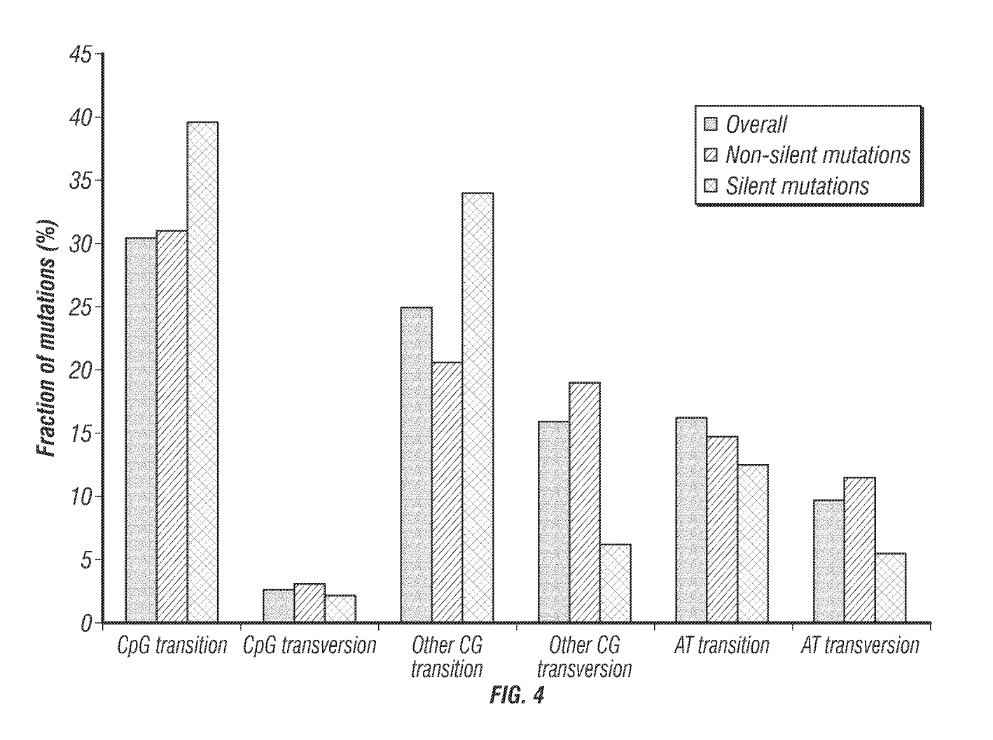

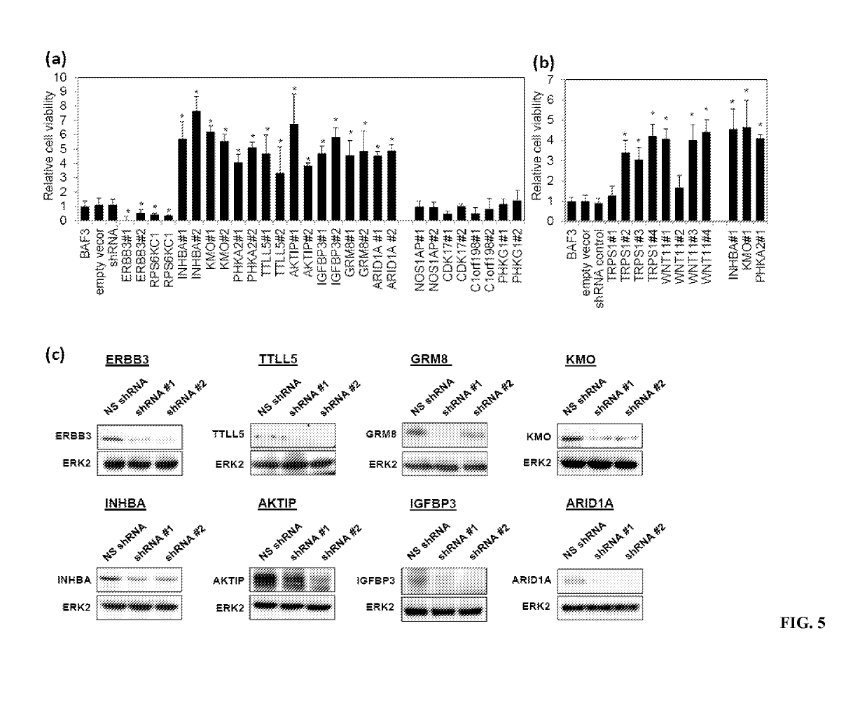

To gain an unbiased view of somatic alterations that contribute to the pathogenesis of endometrial cancer, whole-exome sequencing of 14 endometrial tumor samples (one tumor with unusually high mutation frequency and low estimated tumor content was excluded from further analysis) was performed and normal DNA samples were matched using the SOLiD platform. Table 1 provides the histologic information and other clinical characteristics of these samples. On average, 185 million 50-nucleotide (nt) single reads were obtained for tumor samples and 62 million reads for normal samples (Table 2). The Agilent SureSelect™ capture reagent used for enrichment targets 170,843 exons (˜1.2% of the human genome); for the targeted regions, the average coverage was 94× and 42× for tumor and normal samples, respectively. A computational pipeline was developed to detect somatic mutations by comparing single nucleotide variations in tumor and normal samples (See, e.g., FIGS. 2-3). At the depth of sequencing employed and with the majority of tumors being low grade (grade 2), a mean of 89.5 somatic point mutations (range 34-263) per tumor was identified corresponding to an average of 3.7 mutations per megabase (range 0.98-12.0) (Table 2). As shown in FIG. 4, the most common mutations were transitions in the CpG context (that is, G>A/C>T), which parallels several other cancer lineages (Ding et al. 2010; Chapman et al. 2011; Puente et al. 2011). In addition, the tumors contained a mean of 14.2 somatic coding small insertion/deletions (indels) (See, e.g., Tables 1 and 3).

TABLE 1
Clinical characteristics of patients providing
samples for exome sequencing

| | Histology | | | | |
|---|---|---|---|---|---|
| Sample ID | Subtype | Grade | Stage | Age | Ethnicity |
| E27 | Endometrioid | 3 | IIIc | 66 | Caucasian |
| | & pap serous | | | | |
| E28 | Endometrioid | 3 | IIIc | 83 | Caucasian |
| E35 | Endometrioid | 2 | IIIa | 71 | Caucasian |
| | & pap serous | | | | |
| E58 | Endometrioid | 2 | IIa | 46 | Caucasian |
| E62 | Endometrioid | 2 | IIIc | 71 | Caucasian |
| E70 | Endometrioid | 2 | IIb | 71 | Hispanic |
| E82 | Endometrioid | 2 | Ic | 64 | Hispanic |
| E99 | Endometrioid | 2 | IIIa | 41 | Caucasian |
| E101 | Endometrioid | 2 | IVb | 65 | Caucasian |
| E114 | Endometrioid | 2 | Ib | 73 | Caucasian |
| E161 | Endometrioid | 2 | IVb | 53 | African |
| E170 | Endometrioid | 2 | Ib | 45 | Hispanic |
| E172 | Endometrioid | 2 | Ia | 42 | Hispanic |

TABLE 2
Landscape of somatic mutations in the exomes of 13 endometrial tumors

| | Tumor | Normal | Tumor | Normal | | | | Mutation | |
|---|---|---|---|---|---|---|---|---|---|
| Patient | bases | bases | exome | exome | Callable | All point | Non-silent | rate | Coding |
| ID | sequenced | sequenced | coverage | coverage | positions | mutations | mutations | (per Mb) | Indels |
| E27 | 9.75 × 10⁹ | 4.90 × 10⁹ | 93.0x | 55.0x | 30.9 × 10⁶ | 91 | 49 | 2.95 | 15 |
| E28 | 8.84 × 10⁹ | 4.18 × 10⁹ | 86.3x | 35.8x | 25.1 × 10⁶ | 75 | 48 | 2.99 | 7 |
| E35 | 8.75 × 10⁹ | 4.60 × 10⁹ | 92.0x | 47.4x | 29.0 × 10⁶ | 35 | 19 | 1.21 | 10 |
| E58 | 9.17 × 10⁹ | 4.69 × 10⁹ | 94.1x | 41.7x | 29.1 × 10⁶ | 44 | 24 | 1.51 | 6 |
| E62 | 9.55 × 10⁹ | 2.72 × 10⁹ | 99.6x | 18.7x | 21.9 × 10⁶ | 263 | 146 | 12.0 | 52 |
| E70 | 9.76 × 10⁹ | 4.35 × 10⁹ | 88.3x | 45.2x | 20.7 × 10⁶ | 79 | 43 | 3.82 | 8 |
| E82 | 9.22 × 10⁹ | 4.01 × 10⁹ | 87.1x | 44.8x | 31.2 × 10⁶ | 30 | 13 | 0.96 | 14 |
| E99 | 8.84 × 10⁹ | 4.74 × 10⁹ | 89.2x | 48.5x | 29.9 × 10⁶ | 36 | 17 | 1.20 | 10 |
| E101 | 9.13 × 10⁹ | 4.60 × 10⁹ | 95.7 | 52.8x | 31.3 × 10⁶ | 34 | 18 | 1.09 | 14 |
| E114 | 8.97 × 10⁹ | 4.24 × 10⁹ | 99.6x | 39.0x | 25.9 × 10⁶ | 66 | 43 | 2.54 | 6 |
| E161 | 9.21 × 10⁹ | 4.22 × 10⁹ | 91.6x | 43.3x | 18.7 × 10⁶ | 182 | 81 | 9.74 | 10 |
| E170 | 9.10 × 10⁹ | 4.09 × 10⁹ | 106x | 39.6x | 28.1 × 10⁶ | 188 | 98 | 6.69 | 24 |
| E172 | 9.67 × 10⁹ | 3.53 × 10⁹ | 99.5x | 30.2x | 28.3 × 10⁶ | 41 | 26 | 1.45 | 9 |

Mutations rate was calculated by dividing the total number of somatic mutations by the total number of callable nucleotide positions (≧15x in tumor and ≧8x matched normal samples).

TABLE 3
List of coding indels and mutations in the exomes of 13
endometrial tumors

| | | # | |
|---|---|---|---|
| | # | mut. | Sample |
| Gene | mut | Smpls. | ID |
| ABCC3 | 1 | 1 | E161 |
| ABHD5 | 1 | 1 | E161 |
| ACAD8 | 1 | 1 | E114 |
| ACCN1 | 1 | 1 | E114 |
| ACOT11 | 1 | 1 | E62 |
| ACOT8 | 1 | 1 | E35 |
| ACSS1 | 1 | 1 | E62 |
| ACTN1 | 1 | 1 | E58 |
| ADAD1 | 1 | 1 | E27 |
| ADAM19 | 1 | 1 | E161 |
| ADAM21 | 1 | 1 | E62 |
| ADAMTSL1 | 1 | 1 | E62 |
| ADCY1 | 2 | 2 | E62; |
| | | | E58 |
| ADCY8 | 1 | 1 | E62 |
| ADHFE1 | 1 | 1 | E28 |
| ADNP | 1 | 1 | E161 |
| AFG3L2 | 1 | 1 | E70 |
| AGT | 1 | 1 | E70 |
| AIM1 | 1 | 1 | E114 |
| AKR1C4 | 1 | 1 | E62 |
| AKTIP | 1 | 1 | E27 |
| ALDH1A1 | 1 | 1 | E170 |
| ALOXE3 | 1 | 1 | E58 |
| AMAC1L2 | 1 | 1 | E35 |
| AMDHD1 | 2 | 2 | E170; |
| | | | E27 |
| AMPD3 | 1 | 1 | E35 |
| AMPH | 1 | 1 | E70 |
| AMY2B | 1 | 1 | E114 |
| ANGPT2 | 1 | 1 | E62 |
| ANKMY2 | 1 | 1 | E28 |
| ANKRD30A | 1 | 1 | E28 |
| ANKRD5 | 1 | 1 | E62 |
| ANKZF1 | 1 | 1 | E70 |
| APBA1 | 1 | 1 | E27 |
| APOBEC3H | 1 | 1 | E99 |
| ARFGEF2 | 1 | 1 | E35 |
| ARHGAP20 | 1 | 1 | E62 |
| ARHGEF38 | 1 | 1 | E62 |
| ARID1A | 2 | 2 | E82; E161 |
| ARID3A | 1 | 1 | E62 |
| ARL6IP6 | 1 | 1 | E170 |
| ARSB | 1 | 1 | E62 |
| ASB3 | 1 | 1 | E170 |
| ASB5 | 1 | 1 | E170 |
| ATAD2 | 1 | 1 | E170 |
| ATF6B | 1 | 1 | E170 |
| ATP10D | 1 | 1 | E62 |
| ATRX | 1 | 1 | E161 |
| BCAR1 | 1 | 1 | E82 |
| BCL2L2 | 1 | 1 | E62 |
| BCL9 | 1 | 1 | E170 |
| BCLAF1 | 1 | 1 | E27 |
| BCS1L | 1 | 1 | E70 |
| BEND3 | 1 | 1 | E62 |
| BET1L | 1 | 1 | E170 |
| BRI3BP | 1 | 1 | E99 |
| BTBD7 | 1 | 1 | E170 |
| BTG3 | 1 | 1 | E170 |
| BTN2A1 | 1 | 1 | E170 |
| BTNL2 | 1 | 1 | E161 |
| BUB1 | 1 | 1 | E161 |
| C11orf30 | 1 | 1 | E62 |
| C11orf65 | 1 | 1 | E170 |
| C12orf63 | 1 | 1 | E62 |
| C14orf106 | 1 | 1 | E114 |
| C14orf145 | 1 | 1 | E28 |
| C15orf44 | 1 | 1 | E27 |
| C16orf72 | 1 | 1 | E62 |
| C17orf49 | 1 | 1 | E101 |
| C1orf158 | 1 | 1 | E161 |
| C1orf175 | 1 | 1 | E28 |
| C1orf198 | 1 | 1 | E62 |
| C20orf165 | 1 | 1 | E172 |
| C20orf30 | 1 | 1 | E27 |
| C2orf16 | 1 | 1 | E28 |
| C2orf51 | 1 | 1 | E114 |
| C2orf67 | 1 | 1 | E114 |
| C2orf71 | 1 | 1 | E70 |
| C5 | 1 | 1 | E62 |
| C6 | 1 | 1 | E170 |
| C6orf15 | 1 | 1 | E114 |
| C7orf60 | 1 | 1 | E28 |
| C8A | 1 | 1 | E62 |
| C8B | 1 | 1 | E161 |
| C9 | 1 | 1 | E170 |
| C9orf100 | 1 | 1 | E62 |
| C9orf91 | 1 | 1 | E28 |
| CALCR | 1 | 1 | E170 |
| CAMK2G | 1 | 1 | E101 |
| CAPN9 | 1 | 1 | E170 |
| CASP5 | 1 | 1 | E70 |
| CCDC142 | 1 | 1 | E170 |
| CCDC18 | 1 | 1 | E70 |
| CCT2 | 1 | 1 | E114 |
| CD209 | 1 | 1 | E70 |
| CD63 | 1 | 1 | E170 |
| CDC27 | 1 | 4 | E27; E114; |
| | | | E101; |
| | | | E70 |
| CDC42BPA | 1 | 1 | E28 |
| CDK17 | 1 | 1 | E27 |
| CDKN2AIP | 1 | 1 | E172 |
| CDX4 | 1 | 1 | E62 |
| CEACAM5 | 1 | 1 | E27 |
| CEP120 | 1 | 1 | E161 |
| CEP97 | 1 | 1 | E62 |
| CHD4 | 1 | 1 | E99 |
| CHRM5 | 2 | 2 | E62; E70 |
| CMTM5 | 2 | 2 | E172; E28 |
| CNNM3 | 1 | 1 | E161 |
| CNOT1 | 1 | 1 | E170 |
| CNOT3 | 1 | 1 | E62 |
| CNTN4 | 1 | 1 | E114 |
| CNTN6 | 1 | 1 | E27 |
| CNTNAP1 | 1 | 1 | E161 |
| COG3 | 1 | 1 | E62 |
| COL11A2 | 1 | 1 | E170 |
| COL5A2 | 1 | 1 | E62 |
| COL6A3 | 1 | 1 | E70 |
| CPA3 | 1 | 1 | E70 |
| CPAMD8 | 1 | 1 | E161 |
| CPNE1 | 1 | 1 | E62 |
| CPVL | 1 | 1 | E170 |
| CR2 | 1 | 1 | E62 |
| CSDE1 | 1 | 1 | E170 |
| CTNNB1 | 3 | 5 | E172; |
| | | | E99; |
| | | | E170; E161; |
| | | | E58 |
| CTTN | 1 | 1 | E161 |
| CYLC1 | 1 | 1 | E161 |
| CYP2A6 | 1 | 1 | E62 |
| CYP4B1 | 1 | 1 | E28 |
| CYP4F12 | 1 | 1 | E114 |
| CYP7B1 | 1 | 1 | E27 |
| DBF4B | 1 | 1 | E62 |
| DBX2 | 1 | 1 | E62 |
| DCAF8L1 | 1 | 1 | E161 |
| DCBLD1 | 1 | 1 | E62 |
| DCDC2 | 1 | 1 | E27 |
| DCHS2 | 1 | 1 | E27 |
| DDX54 | 1 | 1 | E27 |
| DENND4C | 1 | 1 | E62 |
| DHX8 | 1 | 1 | E62 |
| DIAPH2 | 1 | 1 | E82 |
| DLGAP3 | 1 | 1 | E172 |
| DMBX1 | 1 | 1 | E161 |
| DMD | 1 | 1 | E161 |
| DNAH3 | 1 | 1 | E62 |
| DNAH5 | 1 | 1 | E170 |
| DNAJC8 | 1 | 1 | E62 |
| DOCK8 | 2 | 2 | E161; E35 |
| DOK2 | 1 | 1 | E101 |
| DPYD | 1 | 1 | E27 |
| DSEL | 1 | 1 | E62 |
| DUSP10 | 1 | 1 | E170 |
| DUSP18 | 1 | 1 | E62 |
| DYNC1LI2 | 1 | 1 | E161 |
| EIF4B | 1 | 1 | E170 |
| EIF4ENIF1 | 1 | 1 | E62 |
| EIF4G2 | 1 | 1 | E170 |
| ELOVL7 | 1 | 1 | E172 |
| EP300 | 1 | 1 | E114 |
| EPHA2 | 1 | 1 | E161 |
| ERBB3 | 1 | 1 | E101 |
| ERBB4 | 1 | 1 | E114 |
| ESRRG | 1 | 1 | E62 |
| EXOC2 | 1 | 1 | E28 |
| EXOC6 | 1 | 1 | E99 |
| EZH1 | 1 | 1 | E170 |
| F5 | 1 | 2 | E28; E70 |
| FAM123A | 1 | 1 | E70 |
| FAM135B | 1 | 1 | E58 |
| FAM155A | 1 | 1 | E170 |
| FAM65A | 1 | 1 | E170 |
| FAM71B | 1 | 1 | E58 |
| FAM89B | 1 | 1 | E170 |
| FAP | 1 | 1 | E62 |
| FARP1 | 1 | 1 | E170 |
| FAT2 | 2 | 2 | E161; E99 |
| FBXO3 | 1 | 1 | E62 |
| FCN1 | 2 | 2 | E62; E28 |
| FCRLA | 1 | 1 | E70 |
| FER1L6 | 1 | 1 | E27 |
| FGF13 | 1 | 1 | E62 |
| FLAD1 | 1 | 1 | E161 |
| FLG | 1 | 1 | E62 |
| FN1 | 1 | 1 | E58 |
| FOXO1 | 1 | 1 | E161 |
| FREM2 | 1 | 1 | E62 |
| FRG2 | 1 | 1 | E62 |
| FRYL | 1 | 1 | E62 |
| FSCB | 1 | 1 | E28 |
| FURIN | 1 | 1 | E62 |
| GABRB1 | 1 | 1 | E27 |
| GAGE12C, GAGE12D, | 1 | 1 | E62 |
| GAGE12E, | | | |
| GAGE12H | | | |
| GALNT5 | 1 | 1 | E28 |
| GDA | 1 | 1 | E114 |
| GEN1 | 1 | 1 | E101 |
| GJB3 | 1 | 1 | E27 |
| GJC3 | 1 | 1 | E62 |
| GLI3 | 1 | 1 | E161 |
| GLRA1 | 1 | 1 | E161 |
| GLRA4 | 1 | 1 | E161 |
| GMEB2 | 1 | 1 | E82 |
| GOLGA3 | 1 | 1 | E170 |
| GOLM1 | 1 | 1 | E99 |
| GPR148 | 1 | 1 | E170 |
| GPR39 | 1 | 1 | E62 |
| GPR61 | 1 | 1 | E62 |
| GPR85 | 1 | 1 | E62 |
| GRIA1 | 1 | 1 | E70 |
| GRIP1 | 1 | 1 | E161 |
| GRM8 | 2 | 2 | E70; E28 |
| GSTA2 | 1 | 1 | E114 |
| GTF3C3 | 1 | 1 | E62 |
| GYG2 | 1 | 1 | E172 |
| HADH | 1 | 1 | E62 |
| HDAC11 | 1 | 1 | E62 |
| HDX | 2 | 2 | E170; E62 |
| HERC2 | 1 | 1 | E170 |
| HHIPL2 | 1 | 1 | E27 |
| HINFP | 1 | 1 | E62 |
| HIST1H3C | 1 | 1 | E28 |
| HIVEP1 | 1 | 1 | E170 |
| HJURP | 1 | 1 | E101 |
| HK2 | 1 | 1 | E62 |
| HLA-C | 1 | 1 | E114 |
| HOXC11 | 1 | 1 | E62 |
| HPSE2 | 1 | 1 | E161 |
| HPX | 1 | 1 | E62 |
| HUWE1 | 1 | 1 | E114 |
| HYDIN | 2 | 1 | E161 |
| ICT1 | 1 | 1 | E70 |
| IFNA4 | 2 | 2 | E114; E58 |
| IFT122 | 1 | 1 | E28 |
| IFT172 | 1 | 1 | E161 |
| IGDCC3 | 1 | 1 | E58 |
| IGF2R | 1 | 1 | E70 |
| IGFBP3 | 1 | 1 | E62 |
| INHBA | 1 | 1 | E62 |
| INTS9 | 1 | 1 | E170 |
| IQCE | 1 | 1 | E170 |
| IREB2 | 1 | 1 | E35 |
| IRF4 | 1 | 1 | E27 |
| IRGC | 1 | 1 | E161 |
| ITGA7 | 1 | 1 | E62 |
| ITGA8 | 1 | 1 | E28 |
| ITGAL | 1 | 1 | E27 |
| ITIH2 | 1 | 1 | E62 |
| JMJD1C | 1 | 1 | E114 |
| KANK1 | 1 | 1 | E28 |
| KCNH1 | 1 | 1 | E62 |
| KCNH6 | 1 | 1 | E58 |
| KCNMB1 | 1 | 1 | E62 |
| KCNT1 | 1 | 1 | E170 |
| KEL | 1 | 1 | E62 |
| KIAA1609 | 1 | 1 | E161 |
| KIAA2022 | 1 | 1 | E82 |
| KIF15 | 1 | 1 | E35 |
| KIF18A | 1 | 1 | E58 |
| KIF2B | 1 | 1 | E70 |
| KIF7 | 1 | 1 | E28 |
| KIFC3 | 1 | 1 | E170 |
| KIR2DL3 | 3 | 3 | E114; E35; |
| | | | E101 |
| KIR3DL1 | 2 | 1 | E27 |
| KIR3DL3 | 1 | 1 | E27; E70 |
| KLK13 | 1 | 1 | E27 |
| KMO | 1 | 1 | E161 |
| KRT75 | 1 | 1 | E161 |
| KRTAP21-2 | 1 | 1 | E27 |
| LAMA3 | 1 | 1 | E99 |
| LAMB4 | 1 | 1 | E27 |
| LAMC2 | 2 | 2 | E114; E62 |
| LATS2 | 1 | 1 | E62 |
| LCT | 1 | 1 | E70 |
| LHFP | 1 | 1 | E28 |
| LPP | 1 | 1 | E82 |
| LRP1B | 1 | 1 | E62 |
| LRRC31 | 1 | 1 | E161 |
| LRRC37B | 1 | 1 | E62 |
| LRRC43 | 1 | 1 | E62 |
| LRRN4 | 1 | 1 | E161 |
| LRTOMT | 2 | 2 | E170; E62 |
| MADD | 1 | 1 | E101 |
| MAEL | 1 | 1 | E62 |
| MAML1 | 1 | 1 | E62 |
| MAP1A | 1 | 1 | E161 |
| MAP2 | 1 | 1 | E172 |
| MAP3K1 | 1 | 1 | E170 |
| MAP4K3 | 1 | 1 | E28 |
| MAVS | 1 | 1 | E99 |
| MB | 1 | 1 | E58 |
| MDC1 | 2 | 2 | E28; E172 |
| MED26 | 1 | 1 | E99 |
| MEFV | 1 | 1 | E27 |
| MEGF10 | 1 | 1 | E172 |
| MFAP5 | 1 | 1 | E27 |
| MFHAS1 | 1 | 1 | E170 |
| MICALCL | 1 | 1 | E62 |
| MID2 | 1 | 1 | E58 |
| MMRN1 | 1 | 1 | E27 |
| MPZL3 | 1 | 1 | E170 |
| MRE11A | 1 | 1 | E161 |
| MTUS1 | 1 | 1 | E28 |
| MYH7 | 1 | 1 | E62 |
| MYH8 | 1 | 1 | E170 |
| MYLK | 1 | 1 | E170 |
| MYLK4 | 1 | 1 | E161 |
| MYO1E | 1 | 1 | E35 |
| MYO5A | 1 | 1 | E62 |
| N4BP3 | 1 | 1 | E172 |
| NAA35 | 1 | 1 | E170 |
| NAPEPLD | 1 | 1 | E170 |
| NBPF10 | 1 | 1 | E99 |
| NBPF9 | 1 | 1 | E161 |
| NCAM2 | 1 | 1 | E28 |
| NCCRP1 | 1 | 1 | E62 |
| NCOA6 | 1 | 1 | E62 |
| NEDD9 | 1 | 1 | E161 |
| NFAT5 | 1 | 1 | E170 |
| NHS | 1 | 1 | E62 |
| NLRP12 | 1 | 1 | E161 |
| NLRP13 | 1 | 1 | E170 |
| NOM1 | 1 | 1 | E170 |
| NOS1AP | 1 | 1 | E27 |
| NRAP | 1 | 1 | E170 |
| NSD1 | 1 | 1 | E62 |
| NUAK2 | 1 | 1 | E161 |
| NUP188 | 1 | 1 | E62 |
| ODZ1 | 1 | 1 | E27 |
| OGDHL | 1 | 1 | E101 |
| OGT | 1 | 1 | E62 |
| OLFM3 | 1 | 1 | E170 |
| OMG | 1 | 1 | E172 |
| OPHN1 | 1 | 1 | E62 |
| OR10A2 | 1 | 1 | E170 |
| OR10G4 | 1 | 1 | E28 |
| OR10G9 | 1 | 1 | E70 |
| OR2B6 | 1 | 1 | E161 |
| OR2G2 | 1 | 1 | E27 |
| OR2T11 | 1 | 1 | E101 |
| OR2T3 | 1 | 1 | E161 |
| OR4A5 | 1 | 1 | E161 |
| OR4D5 | 1 | 1 | E62 |
| OR51B2 | 1 | 1 | E101 |
| OR51E1 | 1 | 1 | E62 |
| OR51T1 | 1 | 1 | E114 |
| OR52A4 | 1 | 1 | E170 |
| OR56B1 | 1 | 1 | E28 |
| OR56B4 | 1 | 1 | E70 |
| OR5AP2 | 1 | 1 | E62 |
| OR5M3 | 1 | 1 | E172 |
| OR6C3 | 1 | 1 | E161 |
| OR6C68 | 1 | 1 | E114 |
| OR7D4 | 1 | 1 | E62 |
| OR8H2 | 1 | 2 | E28; E62 |
| OR8J3 | 1 | 1 | E70 |
| OR9Q1 | 1 | 1 | E161 |
| PABPC1L | 1 | 1 | E58 |
| PADI3 | 1 | 1 | E70 |
| PAN2 | 1 | 1 | E170 |
| PAPOLA | 1 | 1 | E170 |
| PAPPA2 | 1 | 1 | E28 |
| PASD1 | 2 | 1 | E58 |
| PBRM1 | 1 | 1 | E170 |
| PCDH18 | 1 | 1 | E28 |
| PCDH7 | 1 | 1 | E161 |
| PCDHA8 | 1 | 1 | E62 |
| PCDHAC1 | 1 | 1 | E62 |
| PCDHB16 | 1 | 1 | E170 |
| PCSK1 | 1 | 1 | E62 |
| PCYOX1 | 1 | 1 | E27 |
| PDE11A | 1 | 1 | E62 |
| PDE4DIP | 2 | 3 | E35; E70; |
| | | | E161 |
| PDGFRB | 1 | 2 | E35; E58 |
| PDIA2 | 1 | 1 | E101 |
| PEG3,ZI M2 | 1 | 1 | E35 |
| PFKFB3 | 1 | 1 | E62 |
| PHF19 | 1 | 1 | E62 |
| PHKA2 | 1 | 1 | E27 |
| PHKG1 | 1 | 1 | E62 |
| PHLDB2 | 1 | 1 | E27 |
| PIBF1 | 1 | 1 | E170 |
| PIK3CA | 4 | 4 | E170; E114; |
| | | | E172; |
| | | | E28 |
| PIK3CG | 1 | 1 | E170 |
| PIK3R1 | 1 | 1 | E28 |
| PIK3R2 | 1 | 1 | E58 |
| PKM2 | 1 | 1 | E82 |
| PLA2R1 | 2 | 2 | E70; E35 |
| PLAC1 | 1 | 1 | E58 |
| PLCE1 | 1 | 1 | E62 |
| PLXNA1 | 1 | 1 | E170 |
| PLXNA4 | 1 | 1 | E28 |
| PM20D1 | 1 | 1 | E170 |
| PNMAL1 | 1 | 1 | E70 |
| PNP | 1 | 1 | E62 |
| POLD3 | 1 | 1 | E161 |
| POU1F1 | 1 | 1 | E27 |
| PRAMEF1 | 1 | 1 | E114 |
| PRAMEF2 | 1 | 1 | E114 |
| PRB4 | 2 | 6 | E35; E58; |
| | | | E70; |
| | | | E27; E28; |
| | | | E62; |
| PRLR | 1 | 1 | E70 |
| PRSS2 | 1 | 3 | E170; E114; |
| | | | E35 |
| PSD4 | 1 | 1 | E35 |
| PSG2 | 1 | 1 | E161 |
| PSG6 | 2 | 1 | E161 |
| PSMC4 | 1 | 1 | E170 |
| PTEN | 1 | 1 | E170 |
| PTK2 | 1 | 1 | E62 |
| PVRL2 | 1 | 1 | E170 |
| PVRL4 | 1 | 1 | E101 |
| QARS | 1 | 1 | E62 |
| QSER1 | 1 | 1 | E28 |
| R3HDM1 | 1 | 1 | E62 |
| RALGAPB | 1 | 1 | E58 |
| RAVER2 | 1 | 1 | E62 |
| RBM12 | 1 | 1 | E161 |
| RBM12B | 1 | 1 | E28 |
| RFPL3 | 1 | 1 | E161 |
| RFWD3 | 1 | 1 | E161 |
| RGL1 | 1 | 1 | E62 |
| RGS7 | 1 | 1 | E114 |
| RIMS2 | 1 | 1 | E99 |
| RIPK1 | 1 | 1 | E62 |
| RNF10 | 1 | 1 | E170 |
| RNF130 | 1 | 1 | E161 |
| RNF145 | 1 | 1 | E62 |
| RNF17 | 1 | 1 | E99 |
| RNF219 | 1 | 1 | E70 |
| ROPN1B | 1 | 1 | E161 |
| RP1L1 | 1 | 1 | E82 |
| RPE65 | 1 | 1 | E70 |
| RPL18 | 1 | 1 | E170 |
| RPS6KC1 | 1 | 1 | E161 |
| RPTN | 2 | 5 | E28; E114; |
| | | | E172; |
| | | | E82; |
| | | | E62 |
| RSPO1 | 1 | 1 | E161 |
| RTCD1 | 1 | 1 | E161 |
| RTN1 | 1 | 1 | E170 |
| RXFP4 | 1 | 1 | E62 |
| SACS | 1 | 1 | E62 |
| SALL2 | 1 | 1 | E114 |
| SALL3 | 1 | 1 | E27 |
| SAMD4A | 1 | 1 | E27 |
| SCNN1B | 1 | 1 | E161 |
| SEC24D | 1 | 1 | E170 |
| SELPLG | 1 | 1 | E172 |
| SEMA5A | 1 | 1 | E161 |
| SEPT6 | 1 | 1 | E114 |
| SERPINA4 | 1 | 1 | E62 |
| SERPINB10 | 1 | 1 | E170 |
| SFI1 | 1 | 1 | E27 |
| SFTPA1 | 1 | 1 | E62 |
| SGCE | 1 | 1 | E170 |
| SHCBP1 | 1 | 1 | E170 |
| SKIV2L | 1 | 1 | E62 |
| SLC16A5 | 1 | 1 | E62 |
| SLC22A3 | 1 | 1 | E161 |
| SLC30A7 | 1 | 1 | E62 |
| SLC34A3 | 1 | 1 | E172 |
| SLC35A3 | 1 | 1 | E62 |
| SLC38A9 | 1 | 1 | E170 |
| SLC39A5 | 1 | 1 | E58 |
| SLC45A2 | 1 | 1 | E27 |
| SLC4A11 | 1 | 1 | E170 |
| SLC9A7 | 1 | 1 | E99 |
| SLCO3A1 | 1 | 1 | E62 |
| SLIT1 | 1 | 1 | E170 |
| SNRNP200 | 1 | 1 | E170 |
| SNRNP27 | 1 | 1 | E170 |
| SNX21 | 1 | 1 | E161 |
| SNX9 | 1 | 1 | E62 |
| SORL1 | 1 | 1 | E70 |
| SOX30 | 1 | 1 | E62 |
| SPANXN3 | 1 | 1 | E70 |
| SPTB | 1 | 1 | E62 |
| SRD5A3 | 1 | 1 | E62 |
| SRPX | 1 | 1 | E114 |
| SRRM2 | 1 | 1 | E70 |
| SSB | 1 | 1 | E114 |
| STAM2 | 1 | 1 | E170 |
| STK36 | 1 | 1 | E70 |
| STON2 | 1 | 1 | E28 |
| SUFU | 1 | 1 | E101 |
| SUPT6H | 1 | 1 | E170 |
| SUV39H1 | 1 | 1 | E82 |
| SYNE2 | 1 | 1 | E161 |
| SYNPO | 1 | 1 | E114 |
| SYT12 | 1 | 1 | E170 |
| SYTL5 | 1 | 1 | E70 |
| TAF1L | 1 | 1 | E114 |
| TCEAL3 | 1 | 1 | E114 |
| TCEB3B | 1 | 1 | E101 |
| TCF20 | 1 | 1 | E62 |
| TCHH | 2 | 2 | E82; E99 |
| TDG | 1 | 1 | E170 |
| TEKT5 | 1 | 1 | E114 |
| TGM7 | 1 | 1 | E161 |
| TICAM1 | 1 | 1 | E170 |
| TIGIT | 1 | 1 | E161 |
| TKT | 1 | 1 | E35 |
| TLN2 | 1 | 1 | E172 |
| TM6SF1 | 1 | 1 | E27 |
| TMEM146 | 1 | 1 | E170 |
| TMEM31 | 1 | 1 | E161 |
| TMEM48 | 1 | 1 | E27 |
| TMF1 | 1 | 1 | E28 |
| TMPRSS4 | 1 | 1 | E27 |
| TNFRSF10C | 1 | 2 | E172; E62 |
| TNN | 1 | 1 | E161 |
| TNPO1 | 1 | 1 | E170 |
| TP53 | 2 | 2 | E28; E114 |
| TPCN1 | 1 | 1 | E62 |
| TPR | 1 | 1 | E62 |
| TPTE | 1 | 1 | E170 |
| TRA2A | 1 | 1 | E28 |
| TRAF4 | 1 | 1 | E62 |
| TRAF5 | 1 | 1 | E161 |
| TRIM38 | 1 | 1 | E62 |
| TRIM48 | 1 | 1 | E114 |
| TRIP12 | 1 | 1 | E170 |
| TRPC5 | 1 | 1 | E62 |
| TRPS1 | 1 | 1 | E114 |
| TTL | 1 | 1 | E62 |
| TTLL5 | 2 | 2 | E62; E101 |
| UBR4 | 1 | 1 | E172 |
| UBXN10 | 1 | 1 | E62 |
| UGT2A3 | 1 | 1 | E82 |
| UMODL1 | 1 | 1 | E161 |
| URB2 | 1 | 1 | E161 |
| USH2A | 1 | 1 | E62 |
| USP20 | 1 | 1 | E99 |
| USP31 | 1 | 1 | E62 |
| VCAN | 1 | 1 | E70 |
| VPS13A | 1 | 1 | E70 |
| VPS13B | 1 | 1 | E62 |
| VPS53 | 1 | 1 | E62 |
| VSIG1 | 1 | 1 | E62 |
| WDR76 | 1 | 1 | E161 |
| WNT11 | 1 | 1 | E172 |
| WWOX | 1 | 1 | E62 |
| WWP2 | 1 | 1 | E170 |
| XPOT | 1 | 1 | E62 |
| YIPF5 | 1 | 1 | E70 |
| ZBED4 | 1 | 1 | E62 |
| ZBTB20 | 1 | 1 | E58 |
| ZFHX3 | 1 | 1 | E28 |
| ZHX2 | 1 | 1 | E170 |
| ZNF10 | 1 | 1 | E28 |
| ZNF101 | 1 | 1 | E28 |
| ZNF124 | 1 | 1 | E172 |
| ZNF14 | 1 | 1 | E170 |
| ZNF177 | 1 | 1 | E27 |
| ZNF184 | 1 | 1 | E62 |
| ZNF254 | 1 | 1 | E28 |
| ZNF285 | 2 | 2 | E27; E172 |
| ZNF395 | 1 | 1 | E62 |
| ZNF43 | 1 | 1 | E114 |
| ZNF443 | 1 | 1 | E161 |
| ZNF462 | 1 | 1 | E170 |
| ZNF512B | 1 | 1 | E170 |
| ZNF514 | 1 | 1 | E35 |
| ZNF536 | 1 | 1 | E172 |
| ZNF560 | 1 | 1 | E27 |
| ZNF609 | 1 | 1 | E170 |
| ZNF616 | 1 | 1 | E101 |
| ZNF77 | 1 | 1 | E99 |
| ZNF800 | 1 | 1 | E28 |
| ZNF828 | 1 | 1 | E82 |
| ZNF831 | 1 | 1 | E70 |
| ZNFX1 | 2 | 2 | E170; E161 |

Focusing on somatic mutations that potentially affect protein function, across the 13 tumor samples, 576 unique non-synonymous mutations and 24 non-sense mutations were identified in 566 genes (Table 3). The most perturbed biological pathways were integrin, angiopoietin, complement system and PTEN signaling (fisher exact test, P<2.8×10⁻³, false discovery rate [FDR]<0.05, Supplementary Table 3). Based on potential biological interest, we selected 97 mutation sites for Sequenom MASSarray validation and obtained a true positive rate of 81%, leading to 69 genes with validated mutations and, by applying the true positive rate to the remaining aberrations, a prediction of 487 non-synonymous mutations and 18 non-sense mutations in the 13 tumors (Table 4). To estimate the sensitivity of the mutation calling algorithm, we examined full-length sequences of nine genes with concurrent Sanger sequencing and Sequenom® MASSarray detection. The estimated sensitivity was 80%, with false negatives occurring primarily due to low coverage. These results indicate that the identified mutated gene set largely characterizes the landscape of somatic coding mutations in the 13 endometrial cancers assessed.

TABLE 4
Top biological pathways enriched in the mutated genes

| Canonical | | | | |
|---|---|---|---|---|
| Pathways | P-value | FDR | Ratio | Molecules |
| Integrin | 0.00017 | 0.023 | 0.076 | PIK3CA, PIK3R1, ITGA8, BCAR1, |
| Signaling | | | | ITGAL, PTEN, PTK2, MYLK, |
| | | | | TLN2, PIK3CG, CAPN9, PIK3R2, ITGA7, |
| | | | | CTTN, NEDD9, ACTN1 |
| Complement | 0.00028 | 0.023 | 0.18 | C9, C8B, C5, C6, C8A, CR2 |
| System | | | | |
| Angiopoietin | 0.00046 | 0.023 | 0.10 | PTK2, PIK3CA, ANGPT2, FOXO1, |
| Signaling | | | | DOK2, PIK3CG, |
| | | | | PIK3R1, PIK3R2 |
| Lymphotoxin | 0.00077 | 0.024 | 0.12 | PIK3CA, PIK3CG, PIK3R1, TRAF4, |
| β Receptor | | | | TRAF5, PIK3R2, EP300 |
| Signaling | | | | |
| FAK | 0.00078 | 0.024 | 0.091 | PTK2, PIK3CA, TLN2, PIK3CG, PIK3R1, |
| Signaling | | | | CAPN9, PIK3R2, BCAR1, PTEN |
| PTEN | 0.00095 | 0.024 | 0.083 | PTK2, PIK3CA, FOXO1, PIK3CG, PIK3R1, |
| Signaling | | | | PIK3R2, IGF2R, BCAR1, PDGFRB, PTEN |
| Crosstalk | 0.00098 | 0.024 | 0.098 | KIR3DL1, KIR3DL3/LOC100133046, |
| between | | | | KIR2DL3, TLN2, CD209, |
| Dendritic | | | | ITGAL, PVRL2, HLA-C, CAMK2G |
| Cells and | | | | |
| Natural | | | | |
| Killer Cells | | | | |
| NF-κB | 0.0010 | 0.024 | 0.10 | PIK3CA, RIPK1, PIK3CG, PIK3R1, |
| Activation | | | | MAP3K1, PIK3R2, ITGAL, CR2 |
| by Viruses | | | | |
| Sphingosine- | 0.0011 | 0.024 | 0.086 | PTK2, PIK3CA, PLCE1, PIK3CG, PIK3R1, |
| 1-phosphate | | | | ADCY1, PIK3R2, ADCY8, CASP5, PDGFRB |
| Signaling | | | | |
| Leptin | 0.0012 | 0.025 | 0.10 | PIK3CA, PLCE1, FOXO1, PIK3CG, PIK3R1, |
| Signaling in | | | | ADCY1, PIK3R2, ADCY8 |
| Obesity | | | | |
| SAPK/JNK | 0.0013 | 0.025 | 0.089 | MAP4K3, TP53, PIK3CA, RIPK1, DUSP10, |
| Signaling | | | | PIK3CG, PIK3R1, MAP3K1, PIK3R2 |
| TR/RXR | 0.0028 | 0.046 | 0.090 | PIK3CA, COL6A3, NCOA6, PIK3CG, PIK3R1, |
| Activation | | | | PIK3R2, SYT12, EP300 |
| Acute Phase | 0.0028 | 0.046 | 0.070 | PIK3CA, HPX, FN1, RIPK1, ITIH2, PIK3CG, |
| Response | | | | PIK3R1, MAP3K1, C9, C5, PIK3R2, AGT |
| Signaling | | | | |

### Example 2

### Novel Candidate Driver Cancer Genes Revealed by Ba/F3 Functional Assays

Several of the validated mutated genes have previously been shown to be targeted in endometrial cancers, including TP53, PTEN, CTNNB1, PIK3CA, PIK3R1 and PIK3R2 (Lax et al. 2000; Sun et al. 2001; Moreno-Bueno et al. 2002; Oda et al. 2005; Cheung et al. 2011). Their mutation profiles were also reported in a large collection of well-characterized endometrial tumors, demonstrating that aberrations, including frequent co-mutations, of the PI3K pathway occur in ˜80% of endometrial cancers (Cheung et al. 2011). For the remaining mutated genes, their functional roles in endometrial cancer are largely unknown. To identify potential driver cancer genes for further characterization, bioinformatic analyses were performed and 30 genes selected using the following criteria: (i) if the mutation in a gene is predicted to have a high impact on the protein function by CHASM (FDR of impact score<30%) (Carter et al. 2010) or MutAssessor (high impact) (Reva et al. 2011); or (ii) a gene contains multiple or recurrent mutations. Table 5 provides details on selection of candidates for further analysis. The functional impact of these select genes was then evaluated using murine Ba/F3 cells (Warmuth et al. 2007). This cell line is an immortalized murine bone marrow-derived pro-B-cell line that depends on interleukin-3 (IL-3) for growth and proliferation, but readily becomes IL3-independent in the presence of an oncogene or oncogenic event. Thus, the Ba/F3 cells represent a sensitive tool for measuring the effect of an introduced perturbation on cell proliferation and survival, not only for kinase genes but also non-kinase genes (Lutz et al. 2000; Warmuth et al. 2007; Yoda et al. 2010; Cheung et al. 2011).

TABLE 5
List of 30 mutated genes selected for Ba/F3 functional assays

| | | | | Cancer | | |
|---|---|---|---|---|---|---|
| Gene | Multihits | CHASM | FI score | role | Description | Family |
| GRM8 | 1 | | high | | glutamate receptor, metabotropic 8 | G-protein coupled receptor |
| AMDHD1 | 1 | | | | amidohydrolase domain containing 1 | enzyme |
| CHRM5 | 1 | | | | cholinergic receptor, muscarinic 5 | G-protein coupled receptor |
| EPHA2 | 1 | | | | EPH receptor A2 | kinase |
| KMO | 1 | | | | kynurenine 3-monooxygenase | enzyme |
| | | | | | (kynurenine 3-hydroxylase) | |
| NOS1AP | 1 | | | | nitric oxide synthase 1 (neuronal) | other |
| | | | | | adaptor protein | |
| PDE4DIP | 1 | | | | phosphodiesterase 4D interacting | enzyme |
| | | | | | protein | |
| ARID1A | 1 | | | | AT rich interactive domain 1A (SWI- | transcription regulator |
| | | | | | like) | |
| RPS6KC1 | 1 | | | | ribosomal protein S6 kinase | kinase |
| ACCN1 | | 1 | | | amiloride-sensitive cation channel 1, | other |
| | | | | | neuronal | |
| CDK17 | | 1 | | | cyclin-dependent kinase 17 | kinase |
| FCRLA | | 1 | | | Fc receptor-like A | other |
| INHBA | | 1 | | | inhibin, beta A | growth factor |
| LATS2 | | 1 | | | LATS, large tumor suppressor, homolog | kinase |
| | | | | | 2 (Drosophila) | |
| MAP3K1 | | 1 | | | mitogen-activated protein kinase kinase | kinase |
| | | | | | kinase 1 | |
| PHKA2 | | 1 | | | phosphorylase kinase, alpha 2 (liver) | kinase |
| PHKG1 | | 1 | | | phosphorylase kinase, gamma 1 | kinase |
| | | | | | (muscle) | |
| TTLL5 | | 1 | | | tubulin tyrosine ligase-like family, | enzyme |
| | | | | | member 5 | |
| WWP2 | | 1 | | | WW domain containing E3 ubiquitin | enzyme |
| | | | | | protein ligase 2 | |
| EP300 | | | high | | E1A binding protein p300 | transcription regulator |
| ERBB3 | | | high | | v-erb-b2 erythroblastic leukemia viral | kinase |
| | | | | | oncogene homolog 3 | |
| FLAD1 | | | high | | FAD1 flavin adenine dinucleotide | enzyme |
| | | | | | synthetase homolog | |
| PTK2 | | | high | | PTK2 protein tyrosine kinase 2 | kinase |
| TRPS1 | | | high | | trichorhinophalangeal syndrome I | transcription regulator |
| WNT11 | | | high | | wingless-type MMTV integration site | other |
| | | | | | family, member 11 | |
| AKTIP | | | | interacting | AKT interacting protein | other |
| | | | | with AKT | | |
| C1orf198 | | | | novel | chromosome 1 open reading frame 198 | other |
| IGFBP3 | | | | putative | insulin-like growth factor binding | other |
| | | | | tumor | protein 3 | |
| | | | | suppressor | | |
| MFHAS1 | | | | putative | malignant fibrous histiocytoma | other |
| | | | | oncogene | amplified sequence 1 | |
| MTUS1 | | | | putative | microtubule associated tumor | other |
| | | | | tumor | suppressor 1 | |
| | | | | suppressor | | |

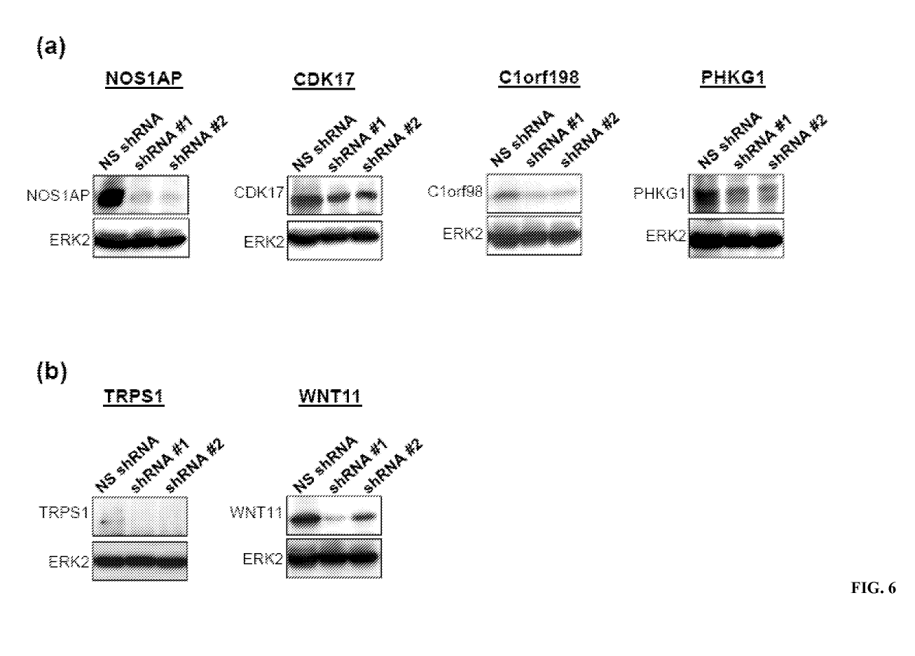

A “loss of function” analysis was first performed on the 30 candidate genes using short-hairpin RNA (shRNA) gene silencing. As shown in FIG. 5a, compared to parental cells and cells transfected with empty vector or non-specific shRNAs, inhibiting the expression of eight genes (INHBA, KMO, PHKA2, TTLL5, AKTIP, IGFBP3, GRM8, and ARID1A) with two independent shRNAs was sufficient to promote the IL3-independent survival of Ba/F3, compatible with these genes having tumor suppressor-like activity. In contrast, the introduction of ERBB3 and RPS6KC1 shRNAs remarkably inhibited IL3-independent cell survival, compatible with oncogene-like activity. These observations were confirmed by at least one of two additional independent shRNAs in a secondary screen. shRNA knockdown efficacy was confirmed by Western blotting for eight proteins where appropriate antibodies were available (FIG. 5c). Nine genes with efficient shRNA knockdown had no effect in the Ba/F3 cells with four (NOS1AP, CDK17, Clorf198 and PHKG1) shown in FIG. 5a and FIG. 6a. One of two shRNAs in the initial screen for two genes (TRPS1 and WNT11) promoted IL3-independent Ba/F3 survival. The activity was confirmed by two additional independent shRNAs (FIG. 5b and FIG. 6b). Significant knockdown was not observed or antibodies were unavailable for the remaining nine genes. Taken together, the shRNA-mediated knockdown screen revealed 12 potential driver cancer genes including 10 tumor suppressors and two oncogene candidates from the 30 genes assessed in the Ba/F3 survival assay.

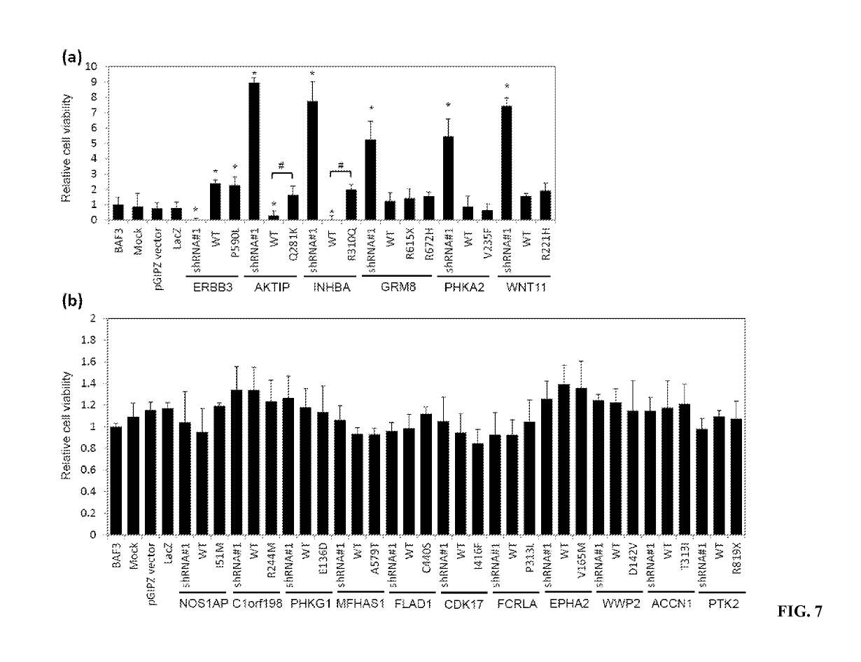

To complement the shRNA screen, a “gain of function” analysis was performed on 17 out of the 30 genes by overexpressing the wild-type or mutated gene in the Ba/F3 cells. Among the 17 genes examined, 6 genes were scored positive in the Ba/F3 shRNA screen. As shown in FIG. 7a, overexpression of wild-type ERBB3 significantly increased cell survival, consistent with its proposed role as an oncogne, but the P590L mutation showed no difference relative to the wild-type, suggesting that this mutation is not functional, albeit in terms of survival in the Ba/F3 assay. Compared with controls, overexpression of wild-type AKTIP and INHBA significantly reduced cell survival, consistent with their potential role as tumor suppressors. Interestingly, their corresponding mutations (Q281K in AKITP and R310Q in INHBA) significantly increased the Ba/F3 survival compared to the wild-type constructs (P<0.05), suggesting a critical inactivating effect of these mutations and strongly supporting AKTIP and INHBA as bonifide tumor suppressors. In contrast, overexpression of GRM8, PHK2, WNT11 or their mutants that scored positive in the shRNA screen showed no significant effect on survival of Ba/F3, suggesting the effects of these genes may be dosage-independent and are not evident in the presence of the wild-type gene in Ba/F3 cells. Concordant with the lack of effect in the Ba/F3 shRNA screen, there was no significant change in Ba/F3 survival with overexpression of the remaining 11 genes or their mutants (FIG. 7b).

### Example 3

### Effect of Candidate Driver Cancer Genes by siRNA Knockdown in Endometrial Cancer Cell Lines

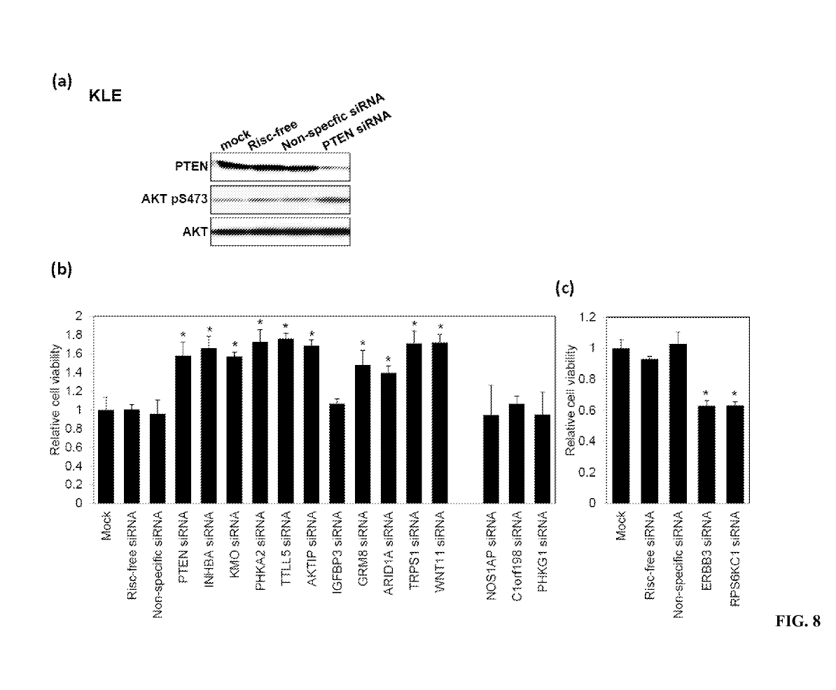

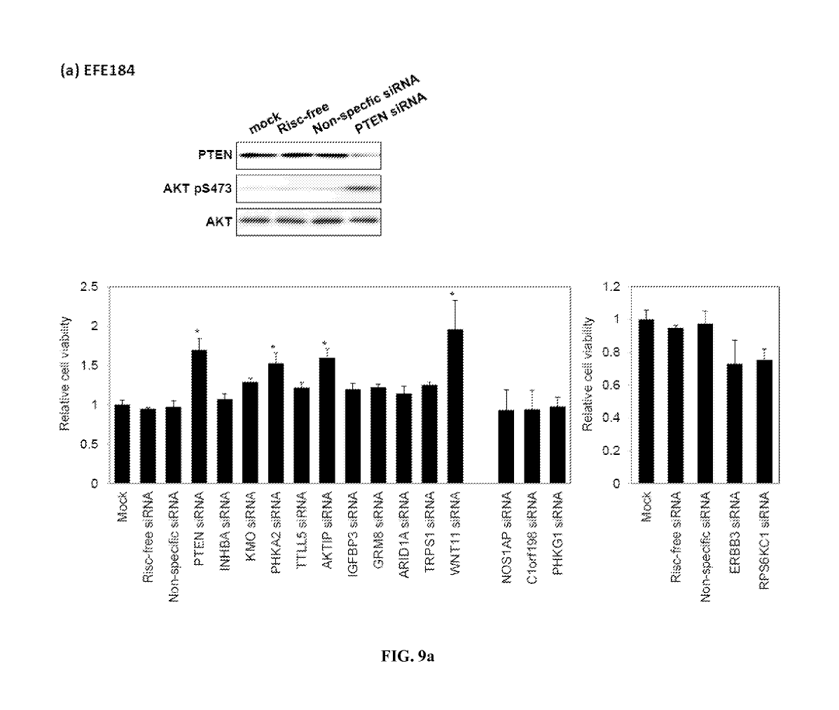

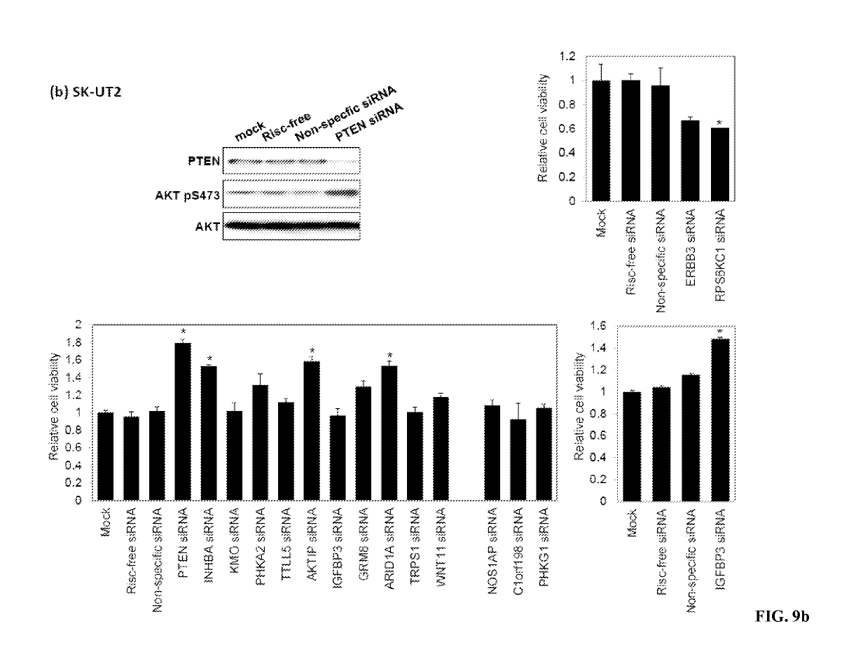

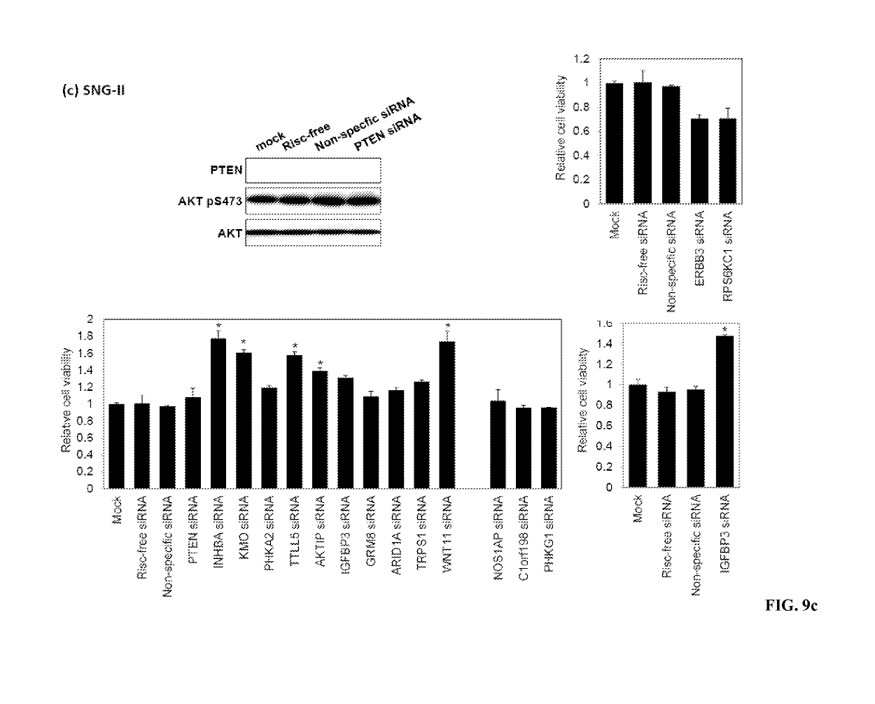

To extend the observations in Ba/F3 cells to human endometrial cancer cells, siRNAs targeting the 12 candidate driver cancer genes were introduced into four human endometrial cancer cell lines and assessed effects on cell viability. A siRNA was included targeting the established PTEN tumor suppressor gene to validate the approach. As shown in FIG. 8 and FIG. 9, decreased PTEN protein and increased AKT phosphorylation was accompanied by significant increased cell viability in PTEN siRNA-transfected cells carrying wild-type PTEN gene and expressing PTEN protein, but not in PTEN protein-negative cell lines, supporting the utility of the approach. Importantly, KLE, in which all genes assessed were wild-type, exhibited the same responses to the corresponding siRNA as Ba/F3 except for IGFBP3 siRNA (FIG. 8b). The responses of EFE184 (all genes tested are wild-type except ARID1A with undetectable protein expression), SK-UT-2 (ARID1A is mutated with detectable protein; mutation status of others is unknown) and SNG-II (ARID1A is mutated with undetectable protein; mutation status of others is unknown) were more variable (FIG. 9). ARID1A siRNA only demonstrated altered viability of KLE and SK-UT-2 where the ARID1A gene is wild type and the protein is expressed (FIG. 8b and FIG. 9). Importantly, AKTIP siRNA significantly increased viability in all cell lines, consistent with its potential tumor suppressor role suggested in the Ba/F3 system. The siRNAs for WNT11 and INHBA increased viability in three cell lines, whereas IGFBP3 siRNA increased cell viability in two cell lines. In contrast, ERBB3 and RPS6KC1 siRNAs inhibited cell viability in all cell lines at least at one time point. siRNAs targeting NOS1AP, PHKG1 and Clorf198, which had no effect in Ba/F3 survival, did not alter cell viability in all the endometrial cell lines examined providing additional support for the validity of Ba/F3 model and the pipeline of characterization of mutations. Collectively, the effect of silencing candidate driver genes in endometrial cancer cell lines largely recapitulated results obtained in Ba/F3 cells.

### Example 4

### Regulation of PI3K Pathway Activation by ARID1A

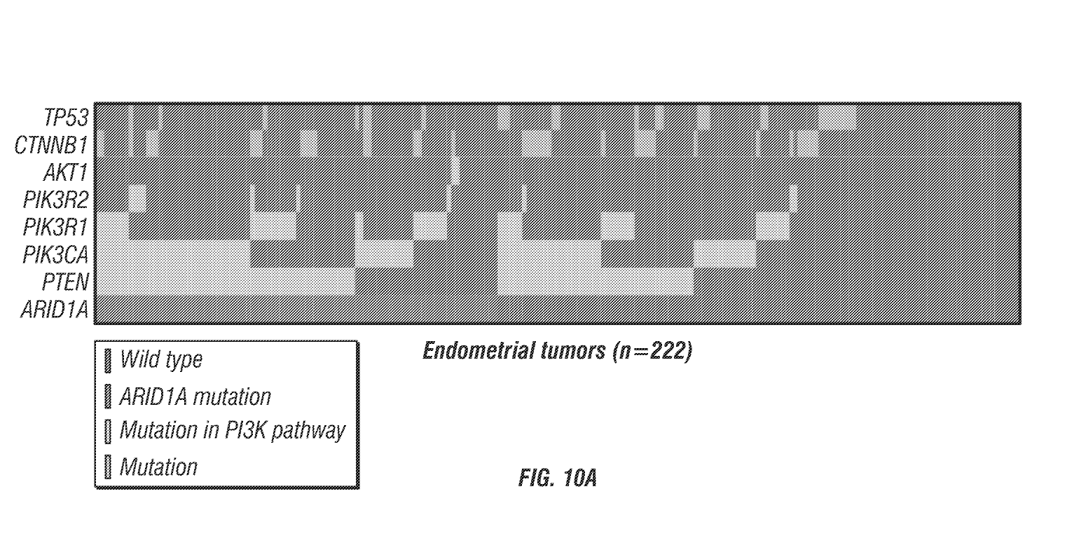

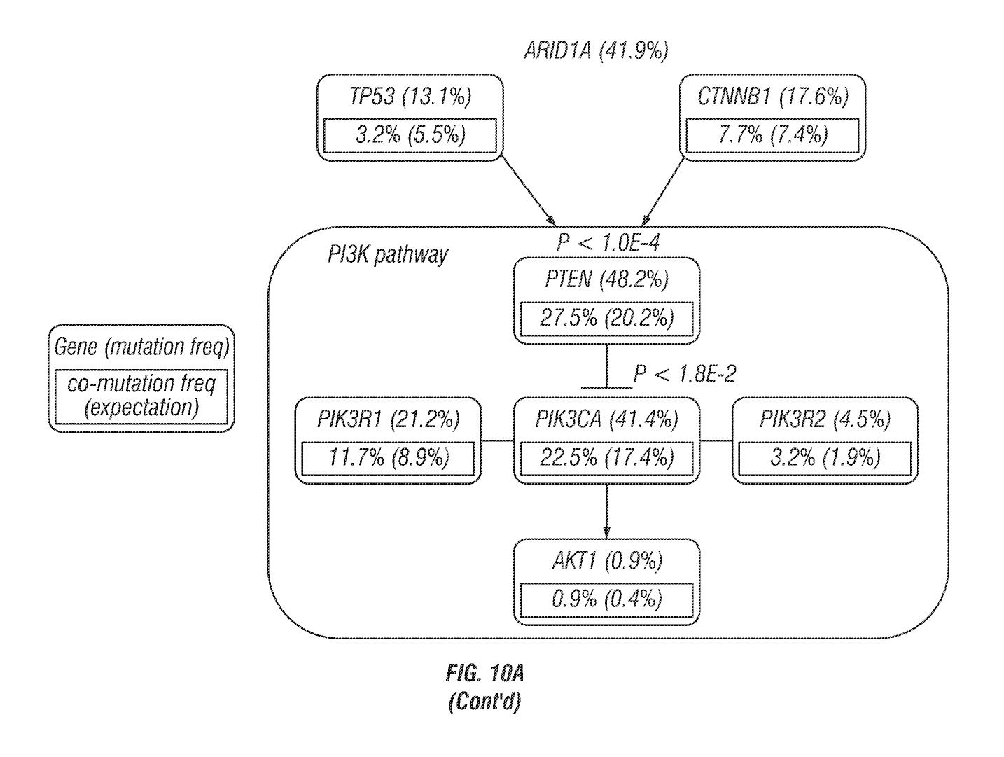

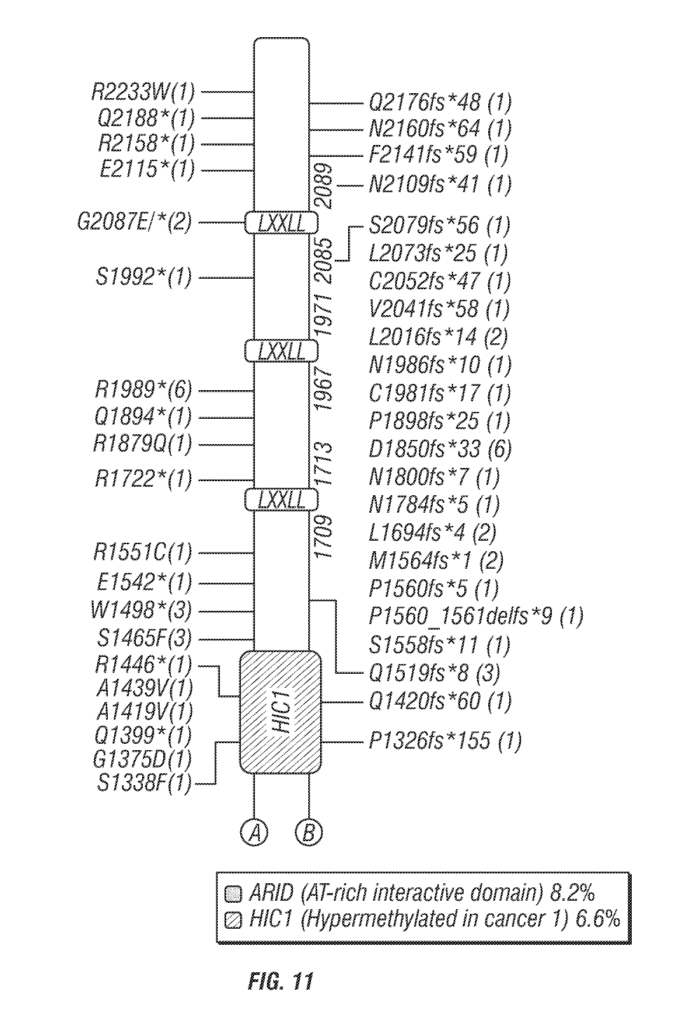

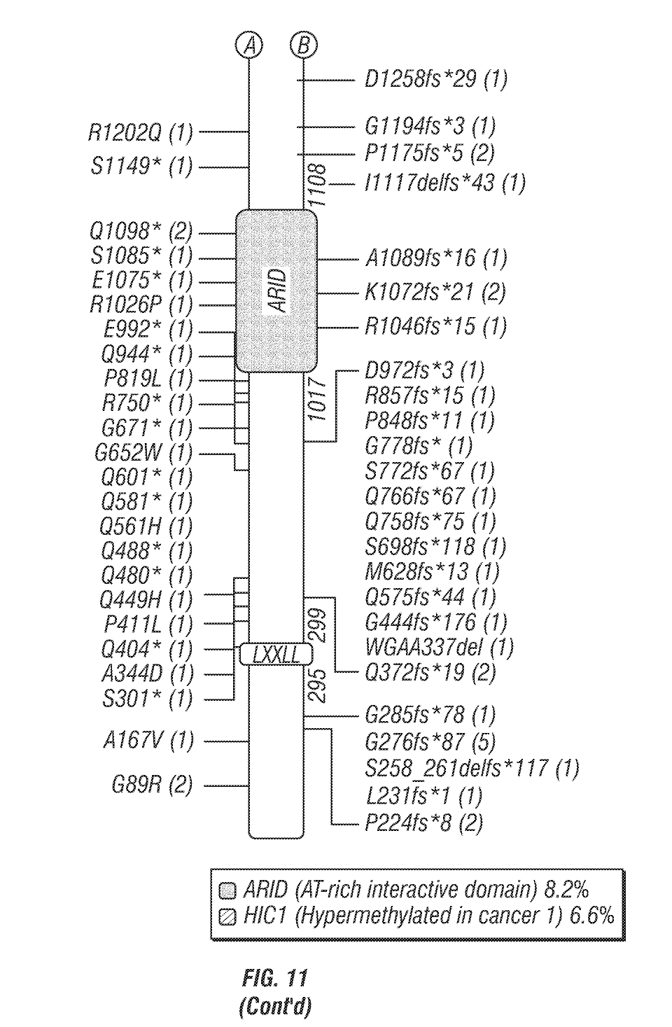

ARID1A (AT-rich interactive domain 1A, also known as BAF250) is a key member of the SWI/SNF chromatin modeling complex has recently been reported to be frequently mutated in a wide variety of cancer types (Jones et al. 2010; Wiegand et al. 2010; Guan et al. 2011b; Gui et al. 2011; Wang et al. 2011; Wiegand et al. 2011; Mamo et al. 2012). While previous studies and the present results suggest its potential role as a tumor suppressor, the molecular mechanism underlying its functional role in cancer is largely unknown. To explore how ARID1A contributes to the pathogenesis of endometrial cancer, the gene was first re-sequenced in the same cohort of tumor samples (n=222) as in the previous study (Cheung et al. 2011) and observed a (non-silent) mutation frequency of 41.6% (FIG. 10a and FIG. 11). Strikingly, ARID1A mutations co-occur with mutations in PTEN (Fisher exact test, P<1.2×10⁻⁵, Bonferroni corrected P<1.0×10⁻⁴) and PIK3CA (P<2.3×10⁻³, Bonferroni corrected P<1.8×10⁻²) (FIG. 6a and Table 6); as well as with overall PI3K pathway aberration (P<1.1×10⁻³, Bonferroni corrected P<8.6×10⁻³). Since coordinate PI3K pathway mutations are common in endometrial cancer relative to other cancer lineages, the concordance of aberrations in the PI3K pathway and ARID1A could represent coordinate targeting of the PI3K pathway or a mutually exclusive function for ARID1A and the PI3K pathway. Thus, the effect of ARID1A mutations on protein and phosphoprotein levels of core members in the PI3K pathway was examined using reverse-phase protein arrays and found that the phosphorylation of several downstream targets (PDK1, AKT, GSK3, TSC2, p70S6K and ACC) are significantly up-regulated in tumors with ARID1A mutations (two-sided t-test, P<0.05, FDR<0.1; FIG. 6b). Since (i) PTEN loss has a dominant effect on the activation of the PI3K pathway (Hollander et al. 2011) and (ii) ARID1A shows co-mutation patterns with PTEN and PIK3CA, studies were focused on a subset of tumor samples in which both PTEN and PIK3CA genes were wild-type, and also PTEN expression was retained (n=47). Strikingly, in these samples, phosphorylation of AKTPS473 and P70S6KPS371, two key PI3K pathway proteins, remain significantly upregulated in AR/D/A-mutated samples (FIG. 10c) as compared to samples where ARID1A, PTEN and PIK3CA are wild type, indicating that the activation of PI3K pathway by ARID1A mutations is not due to co-occurrence of ARID1A mutations with aberrations in PTEN or PIK3CA.

TABLE 6
Histology and mutation information of 222 tumor samples for ARID1A gene re-sequencing

| Sam- | | | | | | | | | |
|---|---|---|---|---|---|---|---|---|---|
| ple | | | | | | | | | |
| ID | Histology | PTEN | PIK3CA | PIK3R1 | PIK3R2 | AKT1 | CTNNB1 | TP53 | ARID1A |
| E1 | Endometrioid 3 | R233STP, | E542K | | | | | | |
| | | Het | | | | | | | |
| E10 | Endometrioid 3 | P95L, Het | | R465_E469del > | | | S37F, Hetero | | |
| | | | | K; | | | | | |
| | | | | Y657fs*6 | | | | | |
| E100 | Endometrioid 3 | E18STP, Het | | | | | | | P1560fs*5 |
| E101 | Endometrioid 2 | | | | | | | | |
| E102 | Mixed-Endo/ | R130G, Het | E545K | | | | | R248W, | |
| | CC | | (Hetero) | | | | | Hetero | |
| E103 | Endometrioid 2 | | Q546R | | | | | | E1075* |
| E104 | Endometrioid 2 | | | | | | | | |
| E106 | Mixed-Endo/ | | | | | | | | |
| | CC | | | | | | | | |
| E107 | Endometrioid 1 | | G118D | | | | | | Q1098* |
| | | | (Hetero) | | | | | | |
| E108 | Endometrioid 2 | P204fs*17, | H1047R/L | | | | | | S1558fs*11; |
| | | het | (Hetero) | | | | | | P1560_1561delfs*9 |
| E109 | Mixed- | | | | | | | | |
| | Endo/Serous | | | | | | | | |
| E11 | Endometrioid 2 | | | | | | | | |
| E110 | Endometrioid 2 | F215fs*6, | | E596fs*66, | | | | | |
| | | het; R130G, | | het; | | | | | |
| | | Het | | 1571fs*31 | | | | | |
| E11 | Endometrioid 2 | Y27N, Het; | E542K | | | | | | G2087* |
| | | R130G, Het | | | | | | | |
| E112 | Endometrioid 2 | | | | | | | | |
| E113 | Mixed- | R173C, Het; | R88Q | R348*, | | | | | R1989* |
| | Endo/CC/ | D92E, Het; | (Hetero) | Het | | | | | |
| | serous | F154L, Het | | | | | | | |
| E114 | Endometrioid 2 | | C420R | | | | | H193R, | |
| | | | | | | | | Homo | |
| E115 | Endometrioid 2 | | | K575fs*23; | | | | | |
| | | | | K382fs*14; | | | | | |
| | | | | K382fs*14 | | | | | |
| E116 | Endometrioid 2 | R173L, Het; | | H450del, | | | | | |
| | | W111STP, | | het | | | | | |
| | | Het | | | | | | | |
| E117 | Endometrioid 2 | Y16fs*28, | | Y467_T471del > | | | | | Q601* |
| | | het | | S; | | | | | |
| | | | | N410fs*8 | | | | | |
| E118 | Endometrioid 1 | K13T, Het; | R93W | R503W, | | | | R213STP, | |
| | | F341C, Het | (Hetero) | Het | | | | Hetero | |
| | | | E453K | | | | | | |
| E119 | Mixed- | | R38S | D578fs*23, | | | | | R2233W; |
| | Endo/CC/ | | (Hetero) | het; | | | | | G285fs*78 |
| | serous | | | D578fs*23, | | | | | |
| | | | | het | | | | | |
| E12 | Endometrioid 2 | | | | | | | | |
| E120 | Endometrioid 2 | | | | | | | | |
| E121 | Endometrioid 2 | | | | | | | | |
| E122 | Endometrioid 2 | | | D578fs*23, | | | | | I1117delfs*43 |
| | | | | het | | | | | |
| E123 | Endometrioid 2 | C71fs*28, | | R503fs*8; | | | S37F, Hetero | | S698fs*118 |
| | | het; | | L30F, | | | | | |
| | | A328fs*16, | | Het; | | | | | |
| | | het | | Q552 > KQ | | | | | |
| E124 | Endometrioid 2 | F341V, Het; | R88Q | E217K, | | | | | |
| | | R130P, Het | (Hetero) | Het; | | | | | |
| | | | D350G | R348STP, | | | | | |
| | | | (Hetero) | Het | | | | | |
| E125 | Endometrioid 2 | K267fs*9, | E39K | | | | S37F | | Q480* |
| | | het; F241S, | (Hetero) | | | | | | |
| | | Het | | | | | | | |
| E126 | Endometrioid 3 | | | | | | | | |
| E127 | Endometrioid 2 | S294fs*13, | H1047Y | | G373R, | | | | D1850fs*33 |
| | | het; G129R, | (Hetero) | | Hetero | | | | |
| | | Het | | | | | | | |
| E129 | Endometrioid 2 | | | | | | | | |
| E13 | Endometrioid 2 | R308fs*10, | | | | | D32Y | L145R, | |
| | | homo | | | | | | Homo | |
| E130 | Serous | | E545K | | | | | | G89R |
| | | | (Hetero) | | | | | | |
| E131 | Endometrioid 2 | K267fs*9, | | | | | | | W1498* |
| | | het | | | | | | | |
| E132 | Mixed- | | | | | | | | W1498* |
| | Endo/CC | | | | | | | | |
| E134 | Mixed 3 | N48del, Het; | E545K | | R35Q, | | SNP (T41A, | | G276fs*87 |
| | | N49S, Homo | (Hetero) | | Hetero | | Hetero+) | | |
| E135 | Mixed- | N323fs*2, | K944fs | | | | | | G1375D |
| | Endo/CC | het; | (Hetero) | | | | | | |
| | | V166fs*14, | C407Y | | | | | | |
| | | het | (Hetero), | | | | | | |
| | | | V344M | | | | | | |
| | | | (Hetero), | | | | | | |
| | | | D350G | | | | | | |
| | | | (Hetero) | | | | | | |
| E136 | clear cell | | | | | | | | G276fs*87 |
| E137 | Mixed- | | | R574del | | | | A189T, | W1498*; |
| | Endo/Serous | | | | | | | hetero | A1419V |
| E138 | Endometrioid 2 | | | | | | | | |
| E139 | Endometrioid 1 | F341C, Het | | | | | S37F | | |
| E14 | Endometrioid 2 | | | | | | | | |
| E141 | Mixed- | | L339I | | | | | | |
| | CC/Serous | | (Hetero) | | | | | | |
| E142 | Endometrioid 2 | | | | | | | | |
| E143 | Mixed- | | H1047R/L | | | | | G279E, | |
| | Endo/Serous | | (Hetero) | | | | | Hetero | |
| E144 | Endometrioid 2 | R130Q, Het | E542V | | | | | | |
| E145 | Endometrioid 2 | | | I442_Y452del | | | | | WGAA337del |
| E146 | Endometrioid 2 | R130G, Het | | | | | | | |
| E147 | Endometrioid 2 | E314STP, | | | | | G34E | | G2087E |
| | | Homo | | | | | | | |
| E148 | Endometrioid 2 | | | | | | | | |
| E149 | Mixed- | | | | | | | | |
| | Endo/Serous | | | | | | | | |
| E15 | Endometrioid 2 | K267fs*9, | Q546K | | | | | | D1850fs*33; |
| | | het; | (Hetero) | | | | | | M1564fs*1 |
| | | N323fs*2, | | | | | | | |
| | | het | | | | | | | |
| E150 | Endometrioid 3 | V170fs*13, | Q546R | | | | | E258STP, | |
| | | het | | | | | | Hetero | |
| E151 | Endometrioid 2 | | | Q579fs*3; | | | | | Q944* |
| | | | | L581fs*2 | | | | | |
| E152 | Endometrioid 2 | Q171R, Het | | K567E, | | | | | Q561H; |
| | | | | Het | | | | | K1072fs*21 |
| E153 | Endometrioid 2 | R233STP, | H1047R/L | | | | | | |
| | | Het; | (Hetero) | | | | | | |
| | | R335STP, | | | | | | | |
| | | Het | | | | | | | |
| E154 | Endometrioid 2 | E201STP, | E453del | | | | | | D1850fs*33 |
| | | Het | (Hetero) | | | | | | |
| E155 | Endometrioid 2 | | | | | | | M246R, | |
| | | | | | | | | Hetero | |
| E156 | Endometrioid 2 | L318fs*2, | | | | | | | S301* |
| | | het | | | | | | | |
| E157 | Mixed- | | | | | | | | |
| | Endo/Serous | | | | | | | | |
| E158 | clear cell | | | | | | | | S1465F |
| E159 | Endometrioid 3 | K267fs*9, | H1047R/L | E297K, | | | | | N1784fs*5 |
| | | het | (Homo); | Het | | | | | |
| | | | H701P | | | | | | |
| E16 | Endometrioid 2 | L247fs*3, | | | | | | | |
| | | het; C124S, | | | | | | | |
| | | Het | | | | | | | |
| E160 | Endometrioid 2 | G251V, | K884R | | | | S37F | | |
| | | Homo | (Hetero) | | | | | | |
| E161 | Endometrioid 2 | | | E451_Y452del | | | S37C | | Y2031* |
| E162 | Endometrioid 2 | R130G, Het; | | | | | SNP (S45F, | | S772fs*67 |
| | | S10del, het | | | | | Hetero+) | | |
| E163 | Endometrioid 2 | R130Q, Het | E542K | | N403I, | | | | Q581* |
| | | | | | Hetero | | | | |
| E164 | Endometrioid 3 | | | RD577del | | | | | A1439V; |
| | | | | | | | | | Q372fs*19 |
| E165 | Endometrioid 3 | E299STP, | | | | | | | S1992* |
| | | Het | | | | | | | |
| E166 | Endometrioid 2 | K267fs*9, | | | | | S37F | | |
| | | het | | | | | | | |
| E167 | Endometrioid 2 | R130G, Het | | H450del | | | | | Q766fs*67 |
| E168 | Endometrioid 2 | | | | | | | | |
| E169 | Mixed-Endo/ | T366fs*44 | | | | | | | V2041fs*58; |
| | CC | | | | | | | | P224fs*8 |
| E17 | Endometrioid 2 | | | | | | | | |
| E170 | Endometrioid 2 | | R88Q | | | | S37F, Hetero | A189T | S1465F, het; |
| | | | (Hetero) | | | | | | E2115*, het |
| E171 | Endometrioid 2 | | | | A171V, | | | | |
| | | | | | Hetero | | | | |
| E172 | Endometrioid 2 | | H1047R/L | | | | SNP (S33C, | | |
| | | | (Homo) | | | | Homo_) | | |
| E173 | Endometrioid 3 | R130G, Het | E110K | | | | | | P1326fs*155 |
| E174 | Mixed-Endo/ | | | | | | | | |
| | CC | | | | | | | | |
| E175 | Endometrioid 2 | N329fs*14, | R38S | | K376N, | | S37F, Hetero | | |
| | | het | (Hetero) | | Hetero | | | | |
| E176 | Endometrioid 3 | E299STP, | | R642STP, | | | | | R1879Q |
| | | Het | | Het; | | | | | |
| | | | | R348STP, | | | | | |
| | | | | Het | | | | | |
| E177 | Endometrioid 3 | V85G, Het | | | | | | R248W, | |
| | | | | | | | | Homo | |
| E178 | Endometrioid 2 | Y336STP, | E453del | E439del | | | | | K1072fs*21 |
| | | Het | (Hetero) | | | | | | |
| E179 | Endometrioid 1 | Y336STP, | R88Q | | | | | | R1989* |
| | | Het | (Hetero) | | | | | | |
| E18 | Endometrioid 3 | R130G, Het | R88Q | | | | | | Q488* |
| E180 | Endometrioid 2 | N323fs*2, | | K142fs*35 | | | SNP (T41I, | | D1850fs*33 |
| | | het; E91*, | | | | | Hetero+) | | |
| | | Het | | | | | | | |
| E181 | Endometrioid 2 | | | | | | | | L2016fs*14; |
| | | | | | | | | | L2073fs*25 |
| E182 | Mixed- | | | I338V, | | | | | |
| | Endo/Serous | | | Het | | | | | |
| E184 | Endometrioid 2 | Y188D, Het; | R357Q | R461STP, | | | | A189T, | |
| | | Y65D, Het; | (Hetero) | Het; | | | | Hetero | |
| | | F341V, Het | | E468STP, | | | | | |
| | | | | Het; | | | | | |
| | | | | R348STP, | | | | | |
| | | | | Het | | | | | |
| E185 | Endometrioid 2 | R130Q, Het | | D605fs*8, | A415T, | | SNP (G34R, | | D972fs*3 |
| | | | | het | Hetero | | Hetero+) | | |
| E186 | Endometrioid 2 | | | K382del; | | | | C135W, | |
| | | | | L380_I381del > F | | | | Hetero | |
| E187 | Mixed- | | M1043V | | | | | | |
| | Endo/Serous 3 | | (Hetero), | | | | | | |
| | | | H1047Y | | | | | | |
| | | | (Hetero) | | | | | | |
| E188 | Endometrioid 2 | R130fs*4, | | | | | | | |
| | | het | | | | | | | |
| E19 | Endometrioid 2 | R130G, Het | | K575fs*26 | | | | | |
| E191 | Endometrioid 2 | | E545K | | | | | | Q372fs*19 |
| | | | (Hetero) | | | | | | |
| E192 | Mixed-Endo/ | | | I442_Y452del | | | | | |
| | CC | | | | | | | | |
| E194 | Endometrioid 2 | L320STP, | R88Q | | | | | | |
| | | Het | (Hetero) | | | | | | |
| E195 | Endometrioid 2 | F37C, Het | H1047R | T576_D578del; | | | S37C | | R750*; |
| | | | | R577_Q579del | | | | | D1258fs*29 |
| E196 | Endometrioid 2 | | | | F118L, | | | | E992* |
| | | | | | Hetero | | | | |
| E197 | Endometrioid 3 | | | | | | | R280T, | |
| | | | | | | | | Homo | |
| E198 | Endometrioid 2 | D24H, Het; | Q546P | | | | | | G652W; |
| | | A72T, Het | (Hetero) | | | | | | G276fs*87 |
| E199 | Endometrioid 1 | | | M582fs*19 | | | Del (TTC, | | C1981fs*17 |
| | | | | | | | Hetero, 273 g) | | |
| E2 | Endometrioid 3 | | | | | | | R282P, | |
| | | | | | | | | Hetero | |
| E20 | Endometrioid 1 | | | | | | | | |
| E200 | Endometrioid 2 | | | | | | | | |
| E201 | Endometrioid 2 | R130G, | | | A36E, | | | | F2141fs*59 |
| | | Hetero | | | Hetero | | | | |
| E202 | Endometrioid 2 | Y16fs*28, | R88Q | | | | | | Q1894* |
| | | het | (Hetero) | | | | | | |
| E203 | Endometrioid 2 | R130Q, | | | | | | | |
| | | Homo | | | | | | | |
| E204 | Endometrioid 2 | | H1047L | | | | | | P224fs*8; |
| | | | (Hetero) | | | | | | Q2176fs*48 |
| E205 | Mixed- | | G118D | | | | | | P1898fs*25 |
| | Endo/Serous | | (Hetero) | | | | | | |
| E206 | Endometrioid 2 | | E545K | | | | | | |
| | | | (Hetero) | | | | | | |
| E207 | Endometrioid 3 | | H1047L | | | | | | Q2188*; |
| | | | (Hetero) | | | | | | S2079fs*56 |
| E208 | Endometrioid 3 | K197fs*2, | | | | | | | |
| | | het | | | | | | | |
| E209 | Endometrioid 2 | R130Q, | H1047? | | | | | | E1542* |
| | | Hetero | | | | | | | |
| E21 | Endometrioid 1 | | | | | | S37F, Hetero | | |
| E210 | Endometrioid | K183fs*7, | | F69L, | | | | | |
| | 1, 2 | het | | Hetero | | | | | |
| E211 | Endometrioid 2 | | | | | | | Del (CT, | |
| | | | | | | | | Hetero, | |
| | | | | | | | | 948) | |
| E213 | Mixed-Endo/ | R142W, | R88Q | D548Q, | | | | | |
| | CC3 | Hetero | (Hetero) | Hetero/ | | | | | |
| | | | | G353R, | | | | | |
| | | | | Hetero | | | | | |
| E214 | Endometrioid 2 | | | | | | | | |
| E215 | Endometrioid 2 | R130G, | R88Q | | | | | | G671* |
| | | Hetero | (Hetero) | | | | | | |
| | | | V344M | | | | | | |
| | | | (Hetero) | | | | | | |
| E216 | Endometrioid | | | | | | | | S1465F |
| | 1, 2 | | | | | | | | |
| E217 | Endometrioid 3 | | R88Q | R503W, | | | | R213STP, | S1338F |
| | | | (Homo) | Hetero/ | | | | Homo | |
| | | | F909L | R348STP, | | | | | |
| | | | (Hetero) | Hetero | | | | | |
| E218 | Endometrioid 2 | T319fs*6, | C420R | | | | | | R1046fs*15 |
| | | het | | | | | | | |
| E219 | Endometrioid 1 | N184fs*6, | E545K | | | | | | N1800fs*7; |
| | | het | (Hetero) | | | | | | L1694fs*4 |
| E22 | Endometrioid 1 | R233STP, | | | | | S33C, Hetero | | |
| | | Het; S33C, | | | | | | | |
| | | Het | | | | | | | |
| E220 | Endometrioid 3 | | E545K | | | | | R273C, | |
| | | | | | | | | Homo | |
| E221 | Endometrioid 2 | | | R639P, Hetero | | | | | |
| E222 | Endometrioid 2 | | | | | | | | |
| E223 | Endometrioid 1 | R130Q, | | | | | | R337H, | |
| | | Hetero | | | | | | Hetero | |
| E224 | Endometrioid 1 | T319fs*6, | | I571 > I | | | | | Q404* |
| | | het | | | | | | | |
| E225 | Endometrioid 1 | | | | | | | | |
| E226 | Endometrioid 1 | R130G | R88Q | T576del; | | | | | C2052fs*47 |
| | | Hetero/ | (Hetero) | T576del | | | | | |
| | | Q219STP, | | | | | | | |
| | | Hetero | | | | | | | |
| E227 | Endometrioid 1 | | | D549fs | | | G34V | | |
| E228 | Endometrioid 2 | | | | | | S45A | | |
| E229 | Endometrioid 2 | A34T, | E545K | | | | | R213STP, | L1694fs*4 |
| | | Hetero; | (Hetero) | | | | | Hetero | |
| | | E288fs*9 | | | | | | | |
| E23 | Endometrioid 1 | | | | | | | | |
| E230 | Endometrioid 2 | | | | | | | | |
| E231 | Endometrioid 2 | K330fs*14, | | | | | D32N | | L231fs*1 |
| | | het | | | | | | | |
| E232 | Endometrioid 2 | R233STP, | R88Q | | A533V, | | | | R1551C; |
| | | Hetero | (Hetero) | | Hetero/ | | | | R1722* |
| | | | H665Q | | S273C, | | | | |
| | | | (Hetero) | | Hetero | | | | |
| | | | R916C | | | | | | |
| | | | (Hetero) | | | | | | |
| | | | C378R | | | | | | |
| | | | (Hetero) | | | | | | |
| E233 | Mixed- | I67K, | R88Q | | P323S, | | T41A | R342STP, | R1989*; |
| | Endo/Serous 3 | Hetero/ | (Hetero) | | Hetero | | | Hetero | Q449H; |
| | | R130Q, | R357Q | | | | | | P1575S |
| | | Hetero | (Hetero) | | | | | | |
| E234 | Endometrioid 3 | | | | | | | | |
| E235 | Endometrioid 2 | | | | | | | | G778fs |
| E237 | Endometrioid 3 | | | | | | | | |
| E238 | Endometrioid 3 | N323fs*6, | | | | | | | D1850fs*33 |
| | | het; | | | | | | | |
| | | L146fs*1 | | | | | | | |
| E239 | Endometrioid 2 | | Q546K | | | | | | |
| | | | (Hetero) | | | | | | |
| E24 | Endometrioid 1 | R130G, Het | K111N | | | | S37F | | |
| E240 | Mixed- | | E110k | | | | S37A; D32Y | Del (T, | G276fs*87; |
| | Endo/Serous 3 | | Y165H | | | | | Hetero | P1175fs*5 |
| | | | (Hetero) | | | | | 693) | |
| | | | I406V | | | | | | |
| | | | (Hetero) | | | | | | |
| E241 | Endometrioid 1 | R130Q, | E545A | | | | D32Y; S37A | | G276fs*87 |
| | | Hetero | (Hetero) | | | | | | |
| E242 | Endometrioid 2 | D92V, | H1047L | | | | D32Y | | |
| | | Hetero | (Hetero) | | | | | | |
| E243 | Endometrioid 1 | L318fs*2, | | | | | D32Y; G34V | | |
| E244 | Endometrioid 1 | R130Q, | R88Q | | | | | | R1989* |
| | | Hetero | (Hetero) | | | | | | |
| E245 | Endometrioid 2 | | | | | | | | |
| E246 | Endometrioid 1 | R130G, | V146I | | | | G34E | | |
| | | Hetero/ | (Hetero) | | | | | | |
| | | Q245STP, | H1047L | | | | | | |
| | | Hetero | (Hetero) | | | | | | |
| E247 | Endometrioid 1 | | | | | | S37C | | |
| E248 | Endometrioid 3 | | | | | | | | |
| E249 | Endometrioid 1 | L318fs*2, | | | | | | | |
| | | homo; | | | | | | | |
| | | T319fs*2, | | | | | | | |
| | | homo; | | | | | | | |
| | | T319fs*2 | | | | | | | |
| E25 | Mixed- | | | | | | | S183STP, | |
| | Endo/Serous | | | | | | | Homo | |
| E250 | Endometrioid 1 | | E545Q | | | | | | |
| E26 | Endometrioid 2 | | | | | | SNP (T41I, | | |
| | | | | | | | Hetero+) | | |
| E27 | Mixed- | | | | | | | | |
| | Endo/Serous | | | | | | | | |
| E28 | Endometrioid 3 | | | I82F, | | | | M237I, | |
| | | | | Hetero | | | | Hetero | |
| E29 | Endometrioid 2 | K13T, Het | R88Q | R724STP, | | | | | R1989*; |
| | | | (Hetero) | Het | | | | | R1202Q |
| | | | M1043I | | | | | | |
| | | | (Hetero) | | | | | | |
| E3 | Endometrioid 3 | F37C, Het | R88Q | I177N, | | | SNP (D32V, | | P411L; |
| | | | | Het | | | Hetero+) | | P819L; |
| | | | | | | | | | R2158* |
| E34 | Serous | | | | | | | | |
| E35 | Mixed- | | | | | | | | A344D |
| | Endo/Serous | | | | | | | | |
| E37 | Serous | | | | | | | Y163H, | |
| | | | | | | | | Homo | |
| E38 | Endometrioid 1 | | | | | E17K | | | G444fs*176 |
| E39 | Endometrioid 2 | | E545K | | | | | | |
| E4 | Endometrioid 3 | R15fs*29, | E110K | | | | | | A167V; |
| | | homo | | | | | | | Q1420fs*60; |
| | | | | | | | | | R857fs*15 |
| E40 | Endometrioid 2 | | | | | | | | |
| E41 | Endometrioid 1 | N323fs*2, | | | | | | | |
| | | het | | | | | | | |
| E42 | Endometrioid 2 | F90fs*9, het; | | N453del, | | | | | |
| | | A126T, Het | | het | | | | | |
| E43 | Endometrioid 2 | | E545K | | | | | | R1026P; |
| | | | (Hetero) | | | | | | Q1399* |
| E49 | clear cell | | | | | | | D281E, | |
| | | | | | | | | Hetero | |
| E51 | Endometrioid 2 | | | | | | D32G | | |
| E52 | Serous (3) | | V344M | | | | | | |
| | | | (Hetero) | | | | | | |
| E55 | Endometrioid 1 | S10fs*1, het | G364R | | | | SNP (T41I, | | G89R; |
| | | | (Hetero), | | | | Hetero+) | | S1085* |
| | | | E365K | | | | | | |
| | | | (Hetero) | | | | | | |
| E58 | Endometrioid 2 | | | | N561D | | S37C | | |
| E6 | Endometrioid 3 | P38S, Het; | R88Q | | | | | | R1446*; |
| | | S59P, Het; | (Hetero) | | | | | | Q758fs*75 |
| | | C136R, Het | | | | | | | |
| E60 | Serous | | | | | | | | |
| E61 | Endometrioid 2 | | | | | | | | |
| E62 | Endometrioid 2 | N323fs*2, | P471L | L449I | | | | | |
| | | het | (Hetero) | | | | | | |
| E63 | Endometrioid 2 | | | | | | | | |
| E64 | Endometrioid 3 | R233STP, | Q546R/P | | | | | | |
| | | Homo | (Hetero) | | | | | | |
| E65 | Endometrioid 2 | R130Q, Het | H1047R | | | | | | |
| | | | (Hetero) | | | | | | |
| E66 | Endometrioid 2 | I101fs*6, het | | | | | | | N1986fs*10 |
| E67 | Endometrioid 2 | N323fs*2, | H1047L | | | | | | Q575fs*44 |
| | | het | | | | | | | |
| E68 | Endometrioid 2 | | | | | | | Y234C, | |
| | | | | | | | | Hetero | |
| E69 | Endometrioid 2 | | | | | | | | N2160fs*64 |
| E7 | Endometrioid 3 | D24G, Het | | | | | SNP (T41I, | | D1850fs*33; |
| | | | | | | | Hetero+) | | A1089fs*16 |
| E70 | Endometrioid 2 | | M1043V | | | | | | |
| | | | (Hetero) | | | | | | |
| | | | E365K | | | | | | |
| | | | (Hetero) | | | | | | |
| E71 | Endometrioid 2 | | | I566 > I, | | | | | |
| | | | | het | | | | | |
| E72 | Endometrioid 2 | | | | | | | | |
| E73 | Endometrioid 2 | R130fs*4, | C420R | | | | S37F, Hetero | | |
| | | het | | | | | | | |
| E74 | Endometrioid 2 | | G118D | | | | | | |
| | | | (Hetero) | | | | | | |
| E75 | Mixed- | | | | | | | F134L, | |
| | Endo/Serous | | | | | | | Homo | |
| E76 | Endometrioid 2 | R130G, Het | E545K | | | | SNP (T41I, | | |
| | | | (Hetero) | | | | Hetero+) | | |
| E77 | Endometrioid 3 | | | | | | | | |
| E79 | Mixed- | | E542V | | | | | | |
| | Endo/Serous | | | | | | | | |
| E8 | Endometrioid 3 | R130G, Het | | H450_E451del | | | | | |
| E80 | Endometrioid 2 | | E545K | | | | | | |
| E81 | Endometrioid 2 | | | | | | | | S1149*; |
| | | | | | | | | | Q1519fs*8 |
| E82 | Endometrioid 2 | | E545K | | | | | | Q1098* |
| E83 | Endometrioid 3 | R15fs*29, | E545K | | | | | | Q1098* |
| | | het; | | | | | | | |
| | | P204fs*17, | | | | | | | |
| | | het | | | | | | | |
| E84 | Endometrioid 2 | | E545K | | | | | | P848fs*11 |
| | | | (Hetero) | | | | | | |
| E85 | Endometrioid 2 | F271C, | | P568 > ALI | | | | | G1194fs*3 |
| | | Homo | | QLRKTR | | | | | |
| | | | | DQYLM | | | | | |
| | | | | KP | | | | | |
| E86 | Endometrioid 2 | A3fs*4, het | H1047L | | | | | | |
| | | | (Hetero) | | | | | | |
| E87 | Endometrioid 2 | N22Y, Het; | | E443_Y452del > | | | | C135W, | S258_261delfs*117 |
| | | L146STP, | | D, | | | | Homo | |
| | | Het | | het | | | | | |
| E88 | Endometrioid 2 | Q245fs*9, | E545K | | | | | | |
| | | het; | | | | | | | |
| | | Y177fs*6 | | | | | | | |
| E89 | Mixed- | E7STP, Het; | D300Y | R557*, | | | | L194F, | |
| | Endo/Serous | D162Y, Het | (Hetero) | Het; | | | | Hetero | |
| | | | R818C | I521M, | | | | | |
| | | | (Hetero) | Het; | | | | | |
| | | | | E458*, | | | | | |
| | | | | Het | | | | | |
| E9 | Endometrioid 3 | “L181_A192delfs*9, | | | | | | | |
| | | het | | | | | | | |
| ″ | G118D | | | | | | | | |
| | (Hetero) | | | | | | | | |
| E90 | Endometrioid 2 | Y225STP, | E545K/A | | | | | | |
| | | Het | (Hetero) | | | | | | |
| E91 | Endometrioid 2 | R15fs*29, | R88Q | S608*, | | | | | R1989* |
| | | het; R173C, | (Hetero), | Het; | | | | | |
| | | Het K163T, | R93W | E160*, | | | | | |
| | | Het; *404L; | (Hetero) | Het | | | | | |
| | | het | | | | | | | |
| E92 | Endometrioid 2 | F215fs*1, | | | | | | | L2016fs*14 |
| | | het; Q214H, | | | | | | | |
| | | Homo | | | | | | | |
| E93 | Endometrioid 2 | | | K567E, | | | | | N2109fs*41 |
| | | | | Het | | | | | |
| E94 | Endometrioid 2 | | | | | | | | |
| E95 | Endometrioid 2 | K267fs*9, | | L449del, | | | | | Q1519fs*8 |
| | | het | | het | | | | | |
| E96 | Endometrioid 3 | L318fs*1, | | E443_Y452del > | | | | | |
| | | het | | D, | | | | | |
| | | | | het | | | | | |
| E97 | Endometrioid 3 | Q17STP, Het | G118D | | | | | | M1564fs*1 |
| | | | (Hetero) | | | | | | |
| | | | D926N | | | | | | |
| | | | (Hetero) | | | | | | |
| E98 | Endometrioid 2 | “K128del, | | | | | | | |
| | | het | | | | | | | |

It was next determined whether ARID1A regulated PI3K pathway activity in endometrial cancer cell lines. Consistent with RPPA analysis from the large endometrial cancer sample cohort, knockdown of ARID1A significantly elevated AKT phosphorylation levels in three cell lines (KLE, ESS1 and MFE280) expressing wild-type ARID1A (FIG. 10d). In contrast, upregulation of AKT phosphorylation was not observed in the EFE184 cell line in which ARID1A protein is not present (FIG. 10d). These results are consistent with inhibition of the PI3K pathway contributing to the tumor suppressor activity of ARID1A.

### DISCUSSION

The foregoing studies represent the first unbiased view of somatic mutations in endometrial cancer, which strongly complements previous gene- and pathway-focused studies. More importantly, the results provide substantial functional evidence for a diversity of novel candidate drivers, suggesting key insights into the pathogenesis of endometrial cancer with implications for the development of targeted therapy.

Next-generation sequencing technology has facilitated characterization of the full spectrum of aberrations in cancer genomes in a cost-effective and timely manner (Mardis 2011). However, there is still a great gap between creating a catalog of mutations and alterations and identifying a short list of “actionable” elements (Chin et al. 2011). Methods described here provide a systems-biology approach to filling this gap: computation-prediction based prioritization was combined with functional screening in a highly sensitive cell viability assay. This approach allows the identification of a large number of candidate driver cancer genes in an efficient way. Focusing on a candidate driver gene of high interest, the underlying molecular mechanisms were further examined through an integrative analysis of mutation profile and protein expression on a large, well-characterized sample set and “hypothesis-driven” functional studies in endometrial cancer cell lines. These methods and the related experimental systems can be readily applied to other cancer types.

Through the combination of gene silencing and overexpression strategies in Ba/F3 and endometrial cancer cell lines, 12 potential driver cancer genes in endometrial cancer were revealed. Among these genes, ARID1A has attracted wide interest recently. Frequent mutations throughout the gene sequence including multiple truncation mutations have been reported in ovarian (Jones et al. 2010; Wiegand et al. 2010), gynecologic (Guan et al. 2011b), bladder (Gui et al. 2011), gastric (Wang et al. 2011), breast (Mamo et al. 2012) and endometrial cancers (Guan et al. 2011a; Wiegand et al. 2011), suggesting a role as a tumor suppressor. ARID1A encodes BAF250a, a nuclear protein and a key component of the SWI/SNF chromatin remodeling complex that functions as a regulator of gene expression and chromatin dynamics (Wu et al. 2009). However, to date, the mechanisms by which loss of ARID1A function contributes to cancer pathophysiology remains poorly understood. ARID1A has been suggested to suppress cell proliferation of ovarian and endometrial cancer cell lines through physically interacting with p53 to coordinately regulate the transcription of cell cycle-related genes (Guan et al. 2011b), linking its function to nuclear localization. Here we not only confirmed a high mutation frequency in a large cohort of endometrial tumors, but also for the first time, demonstrated that ARID1A can regulate PI3K pathway activity, consistent with inhibition of the PI3K pathway contributing to ARID1A tumor suppressor activity. Since the PI3K pathway represents a promising target for therapy (Hennessy et al. 2005), these results have direct implications for clinical translation.

Among the other candidate tumor suppressors identified, AKTIP, INHBA and WNT11, in particular are of great interest. AKTIP and INHBA are particularly likely to represent tumor suppressors due to knockdown and overexpression demonstrating the opposite effects in Ba/F3 and critically, patient derived mutations abrogated the effects of the wild type expression construct (FIGS. 5 and 7). AKTIP was first identified as an AKT binding partner (Remy and Michnick 2004). Exogenous overexpression of AKTIP enhanced AKT phosphorylation but this activation induced apoptosis for unknown reasons (Remy and Michnick 2004). Based on data from the Cancer Genome Atlas, homologous deletion of AKTIP occurs in multiple cancer lineages including breast (2%), ovary (0.9%) and prostate (1%) consistent with a tumor suppressor role. However, the functional role of AKTIP could be tumor type-specific, since it has been proposed as a putative oncoprotein in cervical cancer (Cinghu et al. 2011; Notaridou et al. 2011). INHBA encodes inhibin beta A, a subunit of both the activin and inhibin receptors of the transforming growth factor (TGF-β) superfamily (Risbridger et al. 2001) Inhibin beta A, like TGF-β, can inhibit or stimulate cell growth dependent on the cellular context. For example, INHBA substantially inhibited tumor growth and angiogenesis in in vivo gastric cancer and neuroblastoma models (Schramm et al. 2005; Kaneda et al. 2011). Meanwhile, INHBA mRNA was upregulated in lung cancer and may result in the promotion of cell proliferation (Seder et al. 2009). In endometrial cancer cell lines, the role of INHBA is controversial (Di Simone et al. 2002; Tanaka et al. 2003). It is possible that the contribution of INHBA to the tumorigenesis is determined by the relative expression levels of other receptor subunits and their interacting partners. WNT11 is a key member of the Wingless-type (Wnt) signaling pathway whose de-regulation has been implicated in endometrioid endometrial tumors as evidence by β-catenin mutations and aberrant nuclear accumulation (Ikeda et al. 2000; Moreno-Bueno et al. 2002). The canonical Wnt cascade is mediated by nuclear β-catenin binding to T-cell factor transcription factors to activate genes relevant to tumorigenesis; while non-canonical Wnt signaling is β-catenin independent (Bejsovec 2005). Interestingly, WNT11 is downregulated in hepatocellular carcinoma and it can modulate both canonical and non-canonical Wnt pathways to execute its tumor suppressor actions (Toyama et al. 2010). Thus, the role of WNT11 in Wnt signaling in endometrial cancer warrants further investigation.

ERBB3 and RPS6KC1 are potential oncogenes identified in our study. ERBB3 (HER3) belongs to the epidermal growth factor receptor family and has been implicated in cancer, previously. ERBB2, a partner of ERBB3, as well as ERBB3 itself is often amplified and overexpressed in breast, ovarian, prostate and lung cancers (reviewed in (Sithanandam and Anderson 2008). Overexpression of ERBB3 promotes tumorigenesis through multiple mechanisms including cell cycle progression, stimulation of cell migration and invasion primarily via activation of PI3K pathway (Sithanandam and Anderson 2008). ERBB3 is highly expressed in endometrial cancer but the functional role remains unclear (Srinivasan et al. 1999; Ejskjaer et al. 2007). RPS6KC1 (encoding ribosomal protein S6 kinase polypeptide 1) is another potential oncogene. RPS6KC1 mutations have been previously found in breast, ovary and lung cancers (Davies et al. 2005; Stephens et al. 2005; TCGA 2011). Two independent studies show that RPS6KC1 does not have kinase activity (Hayashi et al. 2002; Liu et al. 2005); but instead, likely functions as an adaptor molecule to recruit binding partners (sphingosine kinase-1 and peroxiredoxin-3) to early endosomes (Hayashi et al. 2002; Liu et al. 2005). These trafficking pathways within the endosomal system play a fundamental role in regulating protein degradation, recycling, secretion and compartmentalization; and indeed defective vesicular trafficking is a hallmark of malignant transformation (Mosesson et al. 2008).

### Example 5

### Identification of PIK3R1 Driver Mutations

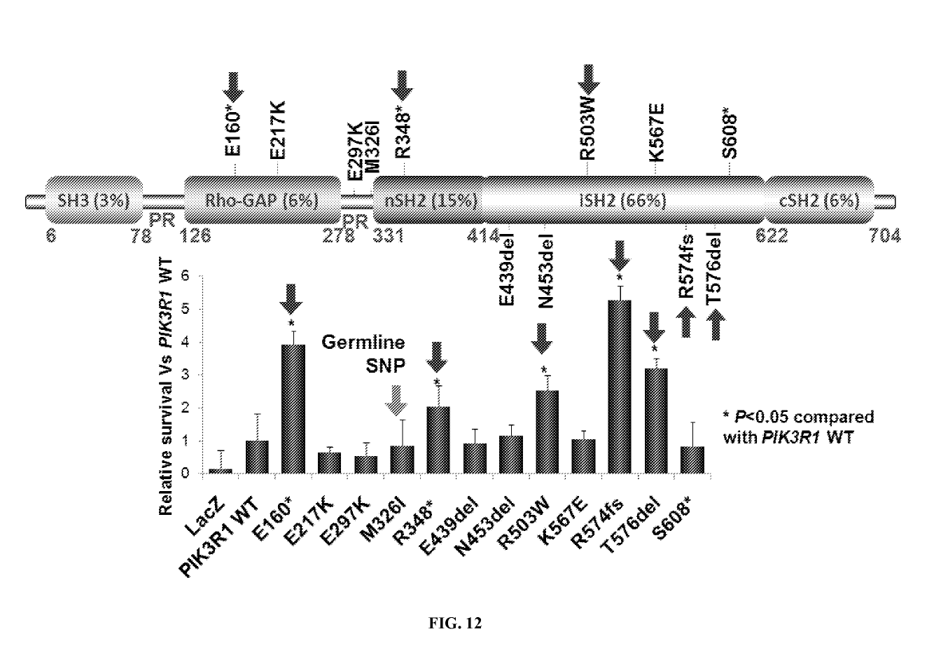

Further tumors were identified with various mutations in PIK3R1. As outlined above mutant coding sequence were cloned into vectors and expressed in Ba/F3 cells to asses which of the mutations may be a driver transformation. As illustrated in FIG. 12, 40% of the mutations in PIK3R1 (the p85 component of phosphatidylinositol 3′kinase (PI3K)) in endometrial cancers were active in the Ba/F3 sensor system. Remarkably, two stop codon mutants, E160* and R348*, that are unable to bind to the p100 catalytic domain of PI3K, increased viability and proliferation of Ba/F3 cells. Further studies indicated that E160* disrupted the formation of p85 homodimers that stabilize PTEN.

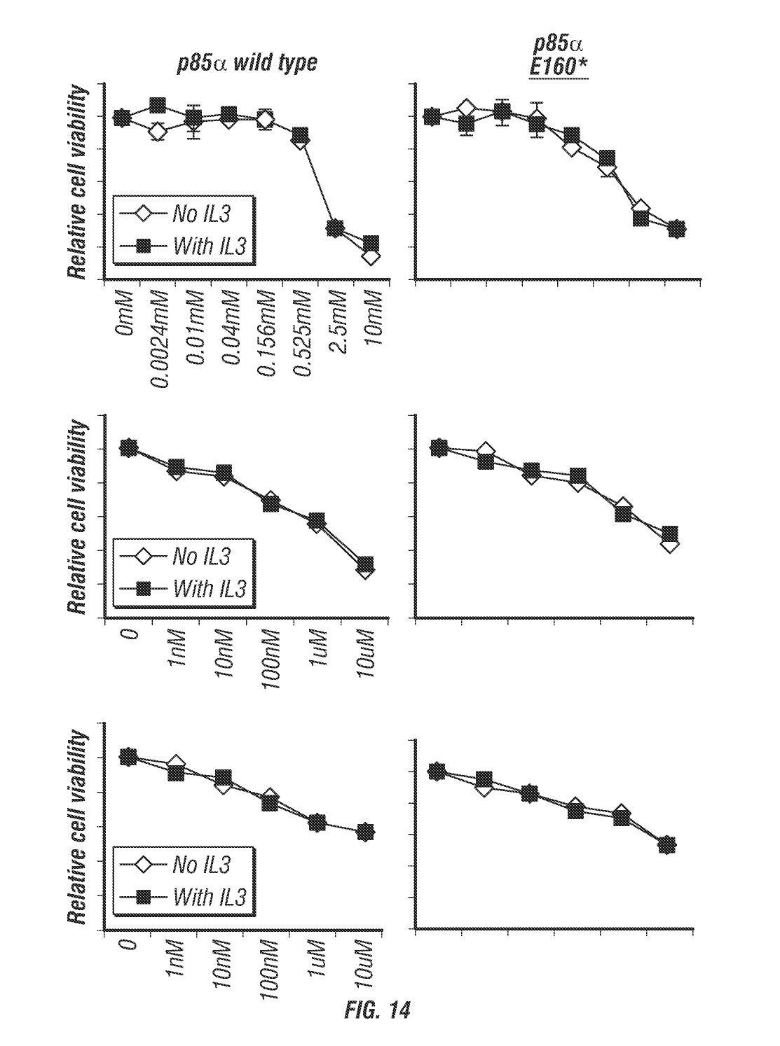

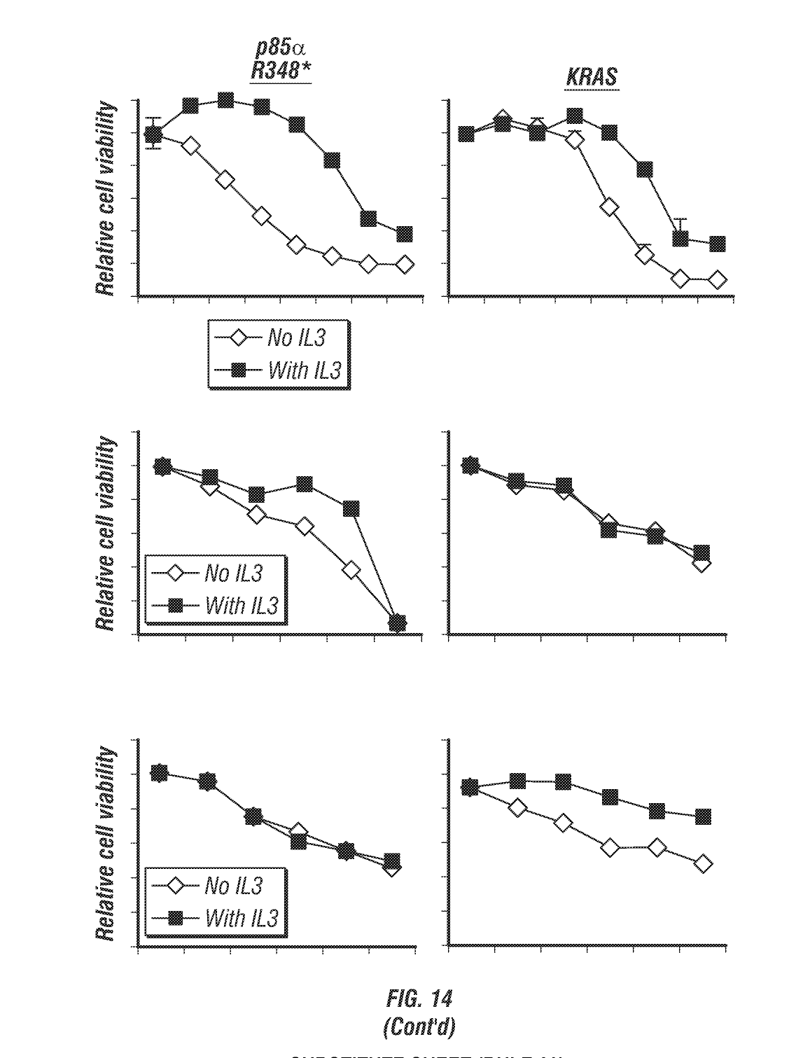

Next, studies were undertaken to determine whether mutations in PIK3R1 or Ras rendered cells any more susceptible to drugs targeting various metabolic pathways. Remarkably, in Ba/F3 cells, R348* p85 as well as oncogenic KRAS were sensitive to 4 independent MEK inhibitors present in the tested drug library (FIG. 14). Surprisingly, p85 R348* was sensitive to 3 independent JNK inhibitors but not p38MAPK inhibitors. In contrast, KRAS demonstrated increased sensitivity to 3 p38MAPK inhibitors but not JNK inhibitors. The use of multiple drugs increases the likelihood that drug actions are on target. This suggested that KRAS induced viability was dependent on activation of erk and p38 but also suggested, surprisingly, that R348* induced viability was dependent on activation of erk, and JNK but not p38. This was completely unexpected as p85 does not normally impinge on the RAS/MAPK pathway.

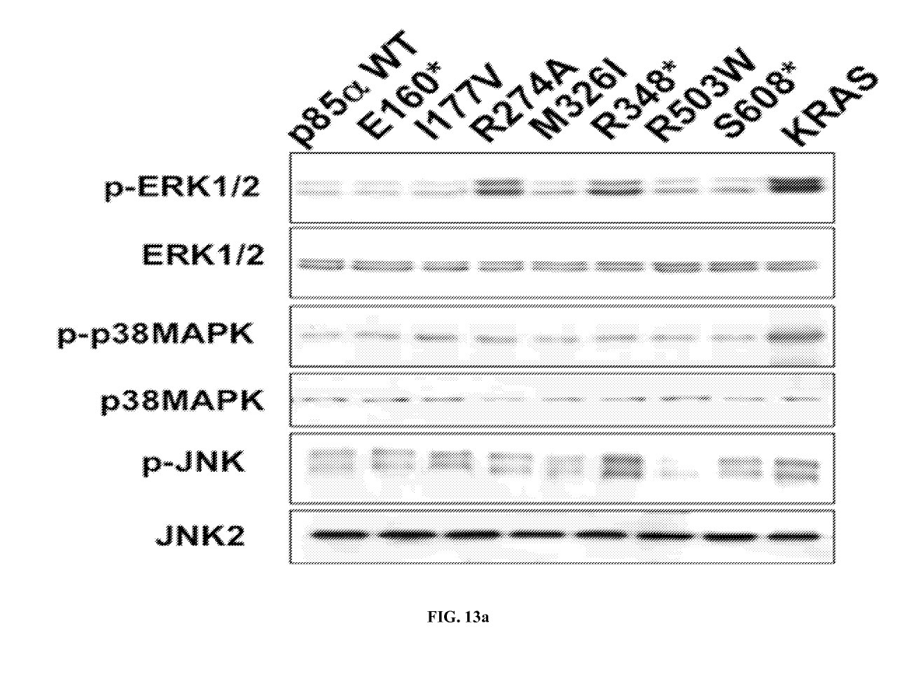

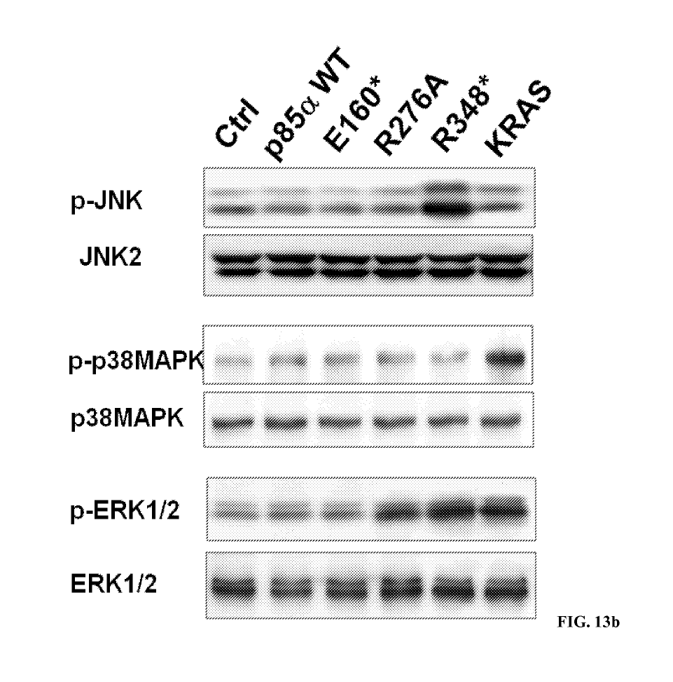

Further studies were completed to confirm the relevant signaling pathways activated by the driver mutations. Such studies could also identify novel metabolic pathways as therapeutic targets for treatment of cancer harboring the mutations. Functional proteomics analyses of the driver aberrations were conducted using Western blot (see, FIG. 13a) and RPPA analysis. These studies demonstrated that KRAS increased MEK, p38 and erk phosphorylation but not JNK phosphorylation as predicted by the drug sensitivity. In complete support of the drug sensitivity data, p85R348* increased phosphorylation of MEK, erk and JNK but not p38. Neither wild type p85 nor multiple other p85 mutations increased ERK, p38 or JNK phosphorylation. These results were recapitulated in SKUT2 endometrial cancer cells (FIG. 13b). Based on these results a detailed mechanism by which p85R348* activates JNK has been elucidated. Importantly, the studies from drug screening and functional proteomics in Ba/F3 are highly concordant and could be recapitulated in epithelial cancer cell lines validating the new pipeline.

### Example 6

### Materials & Methods

### Sample Collection

All studies of 222 patients diagnosed at the University of Texas MD Anderson Cancer Center (Houston, Tex., USA) from 1998 to 2009 were approved by the Institutional Review Board. Tumor content (≧80%), histological classification, grade and stage were reviewed by two independent pathologists. Genomic DNA from frozen tumor resections was extracted by the MD Anderson Bioextraction core using QIAamp DNA Mini Kit (Qiagen); and normal DNA was extracted from peripheral blood leukocytes using QIAamp Blood kit (Qiagen).

### Exome Sequencing

Agilent SureSelect™ Human All Exon Kit (Agilent P/N G3362A) for human exon enrichment and SOLiD fragment library construction kit (Applied Biosystems, P/N 4443471) were used for sequencing library preparation following manufacturers' protocols. The SureSelect™ Human All Exon kit design covers 1.22% of human genomic regions, approximately 38 Mb corresponding to the NCBI Consensus CDS database (CCDS). In brief, 14 endometrial cancer genomic DNA and their paired normal samples were quantitated and qualified individually by nanodrop-1000 (Nanodrop Technologies) and Agilent Bioanalyzer 2100 (Agilent Technologies). Two μg of intact genomic DNA of each sample in 100 μl low TE buffer was fragmented by Covaris S2 into target peak size of 100-150 base pairs (bp). The purification of fragmented genomic DNA was performed using Purelink PCR purification kit (Invitrogen). The fragmented genomic DNA was end repaired using both T4 DNA polymerase and Klenow DNA polymerase at room temperature for 30 min. After purification from end repair mixes, the DNA fragments were ligated with P1 and P2 adaptors on both ends at room temperature for 15 min. Then, a size selection of approximate 200 bp DNA fragments was performed by electrophoresis using E-gel SizeSelect 2% gel (Invitrogen, P/N G661002). The size-selected DNA library (150-200 bp) was amplified by nick translation performed on PCR 9700 thermocycler in 12 cycles using SureSelect pre-capture primers. The PCR products were quantified by Agilent bioanalyzer 2100 DNA 1000 assay. The amplified library demonstrated a peak of size around 200-250 bp.

In the study, 500 ng of each individual library was hybridized with exome capture library for 24 h at 65° C. according to the manufacturer's instructions (Agilent SureSelect Target Enrichment system V1.0). After hybridization, captured targets were enriched by pulling down the biotinylated probe/target hybrids with streptavidin-coated magnetic beads (Dynal magnetic beads, Invitrogen) and purifying the targets with Qiagen MinElute PCR purification columns. Finally, the enriched targeted-DNA libraries were further amplified by post hybridization PCR in 12 cycles and purified by PureLink PCR purification kit. The final library was quantitated by Agilent Bioanalyzer 2100 high sensitivity DNA assay.

The captured exome library was emulsified and amplified individually by emulsion PCR. Full-length template beads were enriched and modified for deposition. The 14 endometrial tumors and paired normal libraries were sequenced in 50 nucleotide (nt) single tags in quad chambers of SOLiD™ V3.0. The tumor samples were sequenced in two quads per sample; and the matched normal samples were sequenced in one quad per sample.

### Detection of Somatic Alternations

FIG. 2 shows the overall computational pipeline for detecting somatic mutations. For each sample, the sequenced reads of 50 nt in color-space from SOLiD™ fragment were primarily analyzed with Applied Biosystems SOLiD™ System Bioscope (version 1.21) software package. The reads were first mapped to the unmasked human reference genome (hg19, genome.ucsc.edu) with default parameters. For targeted regions, single nucleotide variations (SNVs) in tumor samples were called using diBayes module with medium stringency. As an initial quality evaluation, the percentage of inferred SNVs already present in dbSNP (132) was used as an index of SNV calling accuracy. Among the 14 tumor samples, that percentage in one sample (86.1%) was substantially lower than that in the other 13 samples (from 91.1% to 93.9%, a typical range in exome-sequencing literature (Berger et al. 2010). Further analysis on tumor purity based on the contrast of reference allele and novel allele frequency at somatic mutation and polymorphism positions suggested that this sample had the lowest tumor content. Therefore, the tumor sample was excluded from subsequent analysis. SNVs in normal samples were called using diBayes module with low stringency. To reduce false positives in final somatic mutations, additional filters were applied to tumor SNVs: (i) coverage≧15×; (ii) mapping quality of novel alleles (QV)≧11; (iii) the number of novel allele starts≧8×; and (iv) P-value for SNV calling=0. The cut-off value for each parameter was selected based on its effect on the fraction of tumor SNVs that were also called as SNVs in a normal sample, as shown in Supplementary FIG. 2. Accordingly, nucleotide positions with coverage of ≧15× in a tumor and ≧8× in the matched normal were defined as callable positions. To detect somatic mutations, tumor SNVs were filtered by removing those: (i) also called as SNVs in any normal sample; (ii) already present in dbSNP (132)(Sherry et al. 2001) and (iii) common SNPs in the 1000 Genomes Project (1000 Genome Project Consortium 2010). Any tumor SNVs already in the COSMIC database (Bamford et al. 2004) were retained. Small indels (deletions up to 11 bp and insertions up to 3 bp) were called with Find Small Indels Tools in Bioscope with default parameters. Somatic small indels were identified if a tumor indel (i) had a coverage of ≧8× in the matched normal; (ii) not called as an indel in any normal sample; and (iii) not present in dbSNP (132). The functional annotation of somatic mutations or indels was performed with ANNOVAR (Wang et al. 2010).

### Precision and Sensitivity Estimation of Mutation Calling

To estimate the accuracy of the mutation calling method, 97 non-silent somatic mutation sites were selected based on their potential biological interest for Sequenom MASSarray validation. Sequenom assays were designed with the AssayDesign software (version 3.0). The assay design was successful for 95 of the 97 sites. Primers were purchased from Sigma (Houston, Tex., USA) and pooled as indicted by the AssayDesign software. The genomic DNA around the putative SNV was amplified in a multiplex PCR reactions using Qiagen Hotstar Mastermix supplemented with 1.5 mM MgCl₂. Unincorporated primers were removed by Shrimp Alkaline Phosphatase (SAP) digestion at 37C for 40 min, then the SAP was heat inactivated at 85° C. for 5 min. Thermosequenase (Sequenom) was used for primer extension reactions according to Sequenom's standard protocol. All samples were run in duplicate and visually inspected using the Sequenom Typer 3.4 and Typer 4.0 software. In-house software was used to determine whether the putative SNV was confirmed (i.e., the base change was present in the tumor but not the matched normal sample). Of the 95 sites tested, 77 mutations were validated (13 SNPs and 5 wild type), yielding an estimated true positive rate of 81%. Sensitivity is more challenging to assess than precision, owing to the lack of a set of “ground truth” mutations. Re-sequencing of nine genes (CTNNB1, PIK3CA, ARID1A, FRAP1, PIK3CG, PIK3R5, RAB11F1P5, RPS6KC1 and TYRO3) in the tumor samples was performed with Sanger sequencing at the Human Genome Sequencing Center at the Baylor College of Medicine (Houston, Tex.) in order to search for potentially missed somatic mutations. Sequenom analysis was performed for hot spot mutations in PIK3CA to provide additional confidence. Each novel (non-silent) tumor SNV in these genes was further genotyped by Sequenom in both tumor and the matched normal samples to determine whether it was a “true” somatic mutation. With this approach, there were 15 somatic mutations identified in the callable positions of these genes in total, and 12 were also detected by SOLiD, yielding an estimated sensitivity of 80%.

### Mutational Analysis

Mutation data of ARID1A in 222 endometrial tumors were obtained as previously described (Cheung et al. 2011). Two-sided fisher exact test was used to assess whether (non-silent) mutations in ARID1A tend to co-occur with mutations in other genes (or PI3K pathway aberrations) in the tumor samples. PI3K pathway aberrations were defined as PTEN loss or mutations in any pathway genes (including PTEN, PIK3CA, PIK3R1, PIK3R2 and AKT1). Bonferroni correction was used to control multiple testing.

### RPPA Data Analysis

To examine the effect of ARID1A mutations on PI3K pathway, high-throughput RPPA data for 24 PI3K-pathway-related proteins in the 222 samples were obtained from our previous study (Cheung et al. 2011). RPPA data was processed and normalized as previously described (Park et al. 2010). Two-sided t-test was used to test whether the expression of a given protein in ARID1A wild-type samples was significantly different from that in ARID1A mutated samples, and false discovery rate (Benjamini and Hochberg 1995) was used to control multiple testing.

### Bioinformatics Analysis

Pathway enrichment analysis was performed with Ingenuity Pathways Analysis (IPA) (version 8.0). The status of PTEN was determined as in our previous study (Cheung et al. 2011). Two-sided Fisher exact test was used to assess whether ARID1A mutations were correlated with the status of PTEN (loss or retained). To select mutated genes for shRNA studies, functional effects of somatic mutations were predicted with two programs: (i) CHASM (Carter et al. 2010), mutations with FDR<30% were selected; (ii) MutAssessor (Reva et al. 2011), mutations with high functional effect were selected.

### Ba/F3 Viability Assays

The interleukin-3 (IL-3)-dependent prolymphoid cell line Ba/F3 was maintained in RPMI1640 medium containing 10% fetal bovine serum supplemented with 5 ng/ml IL-3 at 37° C. in a 5% CO₂ atmosphere. Constructs containing wild-type genes or their corresponding mutants cloned into pLenti6.3 (Invitrogen) and shRNAs in pGIPZ vector (Open Biosystems) were transfected into Ba/F3 cells using the Neon electroporation system according to manufacturer's instruction (Invitrogen). At 96 h posttransfection, the cells were resuspended in medium without IL-3. Cells (5×10³) were plated in 96-well plate and were cultured for 4 weeks. Cell viability was evaluated using Cell Titre Blue (Promega) for mitochondrial dehydrogenase activity. Statistical analysis was performed using ANOVA followed by Tukey's post hoc test (GraphPad Software, San Diego, Calif.). P<0.05 was considered statistically significant.

siRNA Transfection in Endometrial Cancer Cell Lines

Endometrial cell lines EFE-184, ESS-1 and MFE280 were purchased from DSMZ-German Collection of Microorganisms and Cell Cultures (Braunschweig, Germany). KLE was provided by Dr. Russell Broaddus (MD Anderson Cancer Center, Houston, Tex., USA), and SK-UT-2 was kindly provided by Dr. Bo R. Rueda (Massachusetts General Hospital, Boston, Mass., USA). All cell lines were cultured in media according to the suppliers' instructions at 37° C. in a 5% CO₂ atmosphere. Cells were transfected with 20 nM siRNAs (Dharmacon) using Lipofectamine RNAiMAX reagent (Invitrogen) according to the manufacturers' instructions. The cells were harvested at time points as indicated in the figure legends and processed for either cell viability assay using Cell Titre Blue (Promega) or Western blot analysis.

### Western Blotting

Whole cell lysates were extracted with RIPA lysis buffer (1% NP40, 5 mM EDTA, 1 mM sodium orthovanadate, 1% phenylmethylsulphonyl fluoride and complete protease inhibitor cocktail). The protein concentrations were determined with DC Protein Assay Reagent (Bio-Rad Laboratories). Cell lysates (25 μg) were loaded onto SDS/PAGE and transferred to Hybond-ECL nitrocellulose membrane (Amersham Biosciences). The membrane was blocked with 5% nonfat milk and incubated with primary antibody at 4° C. overnight. Protein expression was visualized with ECL plus kit (Amersham Biosciences).

### Example 7

### Identification of RNA Editing Sites

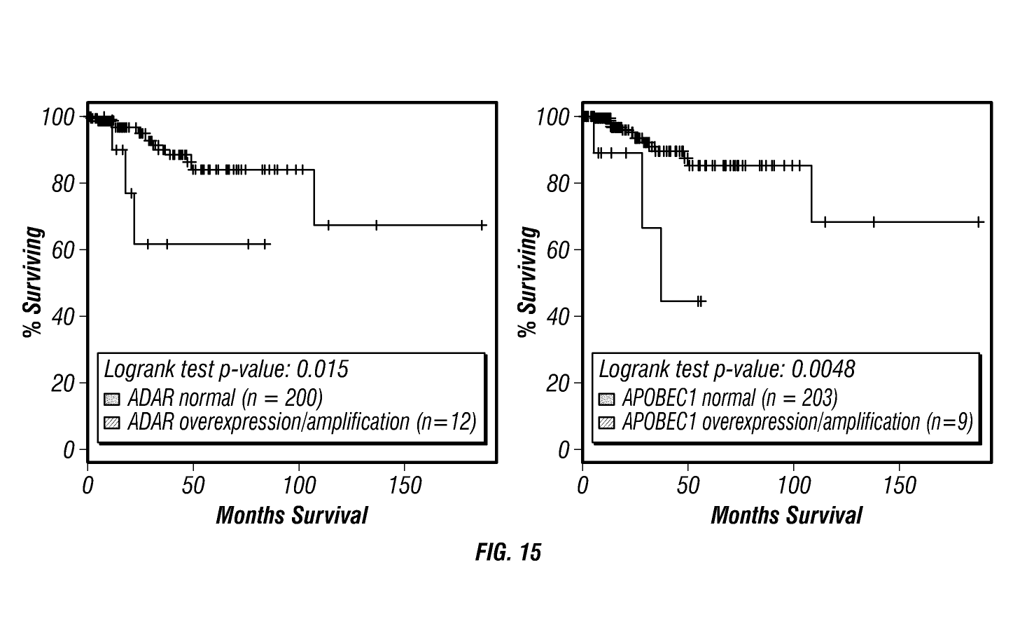

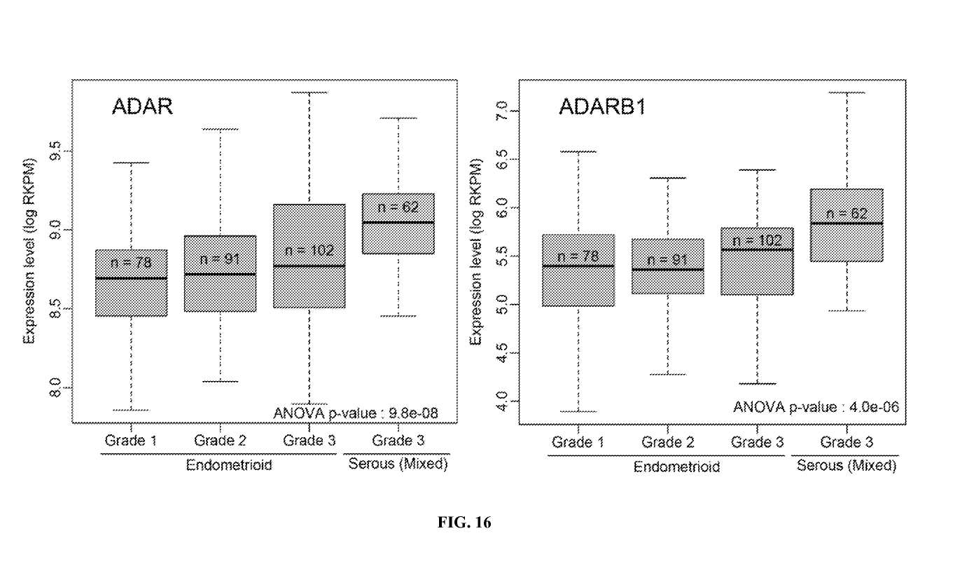

FIG. 15 shows Kaplan-Meier survival curves illustrating the correlation between RNA editing enzymes and endometrial cancer patient survival. Both ADAR overexpression/amplification and APOBEC1 overexpression/amplification correlated with a significant decrease in survival. RNA editing enzymes also show differential expression among endometrial tumor subtypes (FIG. 16). ADAR and ADARB1 were found to be expressed at a higher level in serous Grade 3 tumors that represent the most aggressive endometrial cancers.

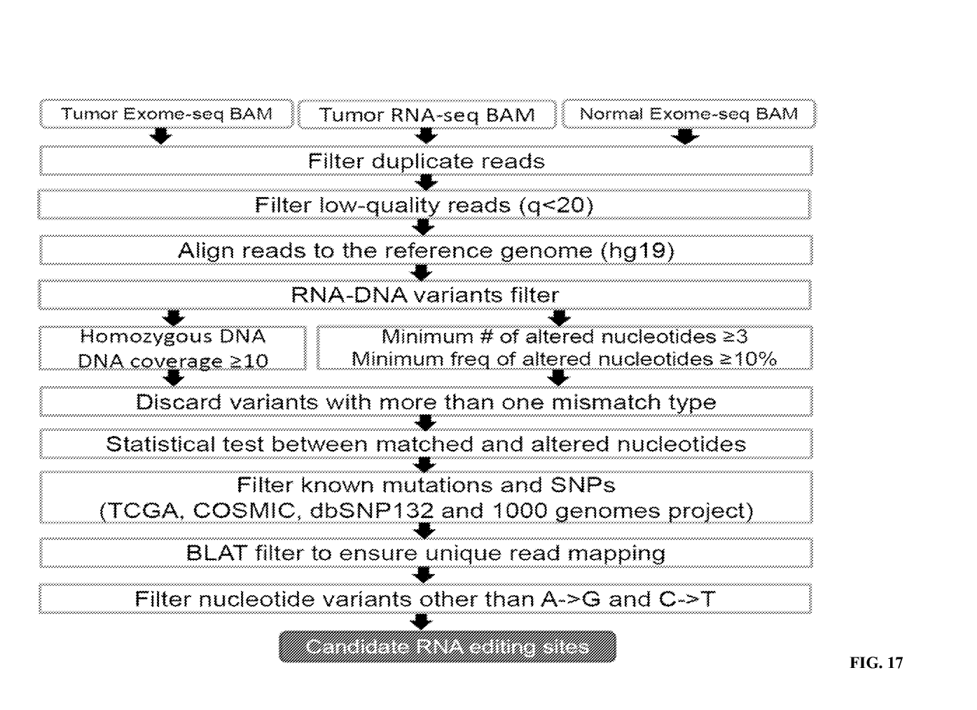

The inventors built a computational pipeline to identify RNA editing events from sequencing data (FIG. 17). The inventors applied this pipeline to 161 TCGA endometrial tumors using both RNA-seq and exome-seq data. Sixty-four highly recurrent RNA editing sites were identified in endometrial cancer. Sequenome validation on an independent sample set yielded a validation rate greater than 70%.

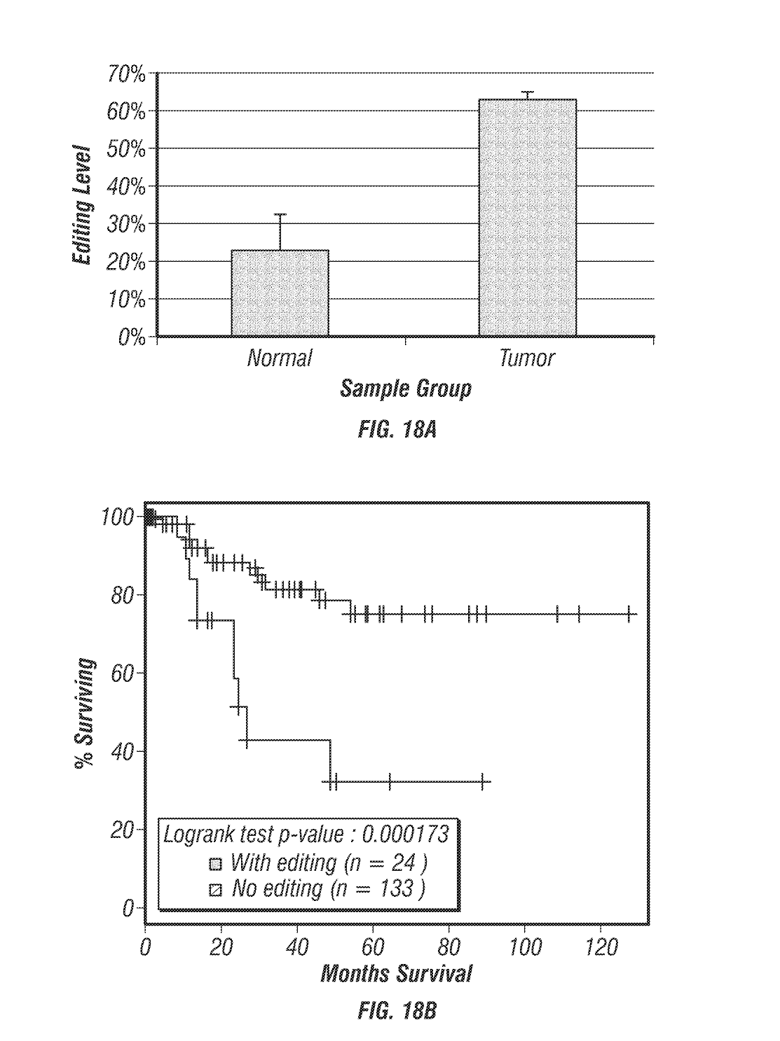

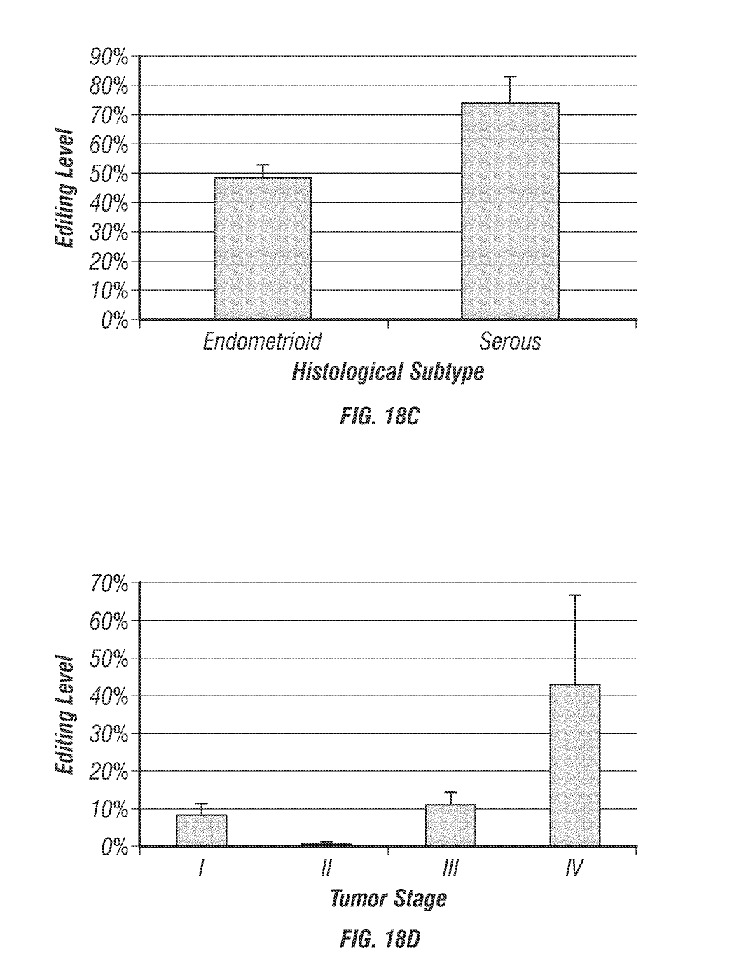

Representative examples of validated RNA editing sites include A-to-I editing at chr17_55478740 in MSI2 and at chr4_15881294 in GRIA2. The inventors analyzed the editing level of representative RNA editing sites with a potential functional role (FIG. 18). The editing level was elevated in the tumor sample group relative to the normal sample group (FIG. 18A). Survival was decreased in the population with editing relative to the population without editing (FIG. 18B). The editing level was elevated in the serous histological subtype relative to the endometrioid subtype (FIG. 18C). Finally, the editing level was elevated in stage IV tumors relative to stage I-III tumors (FIG. 18D).

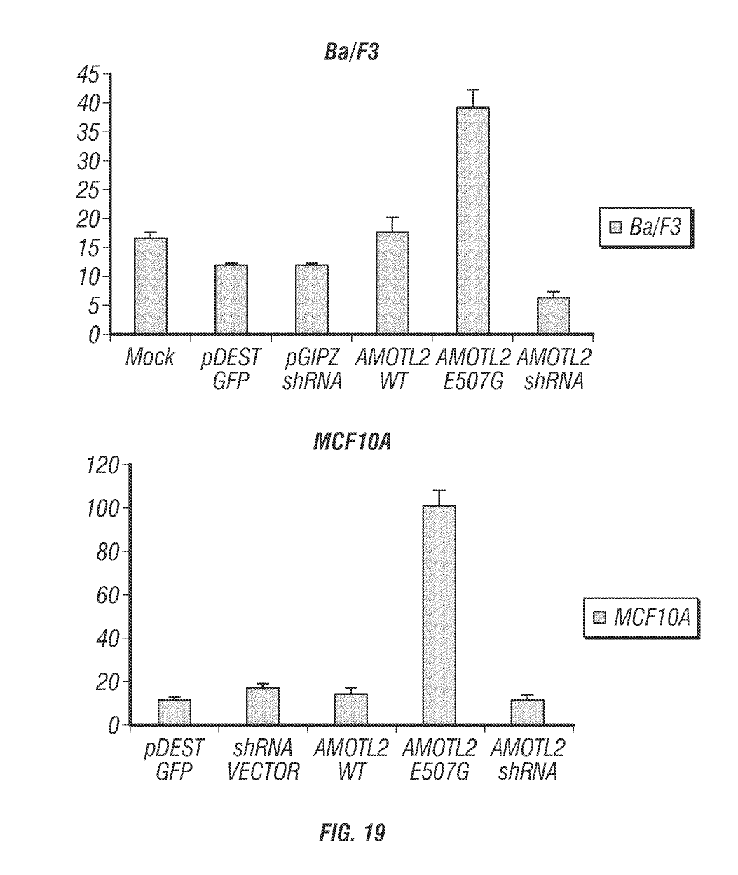

In view of the potential role of RNA editing in cell transformation additional studies were undertaken using sensor cell lines to determine if RNA editing events could result in the generation of driver mutations in effected genes. In this study AMOTL2 edited variant E507G was expressed along with multiple controls in two sensor cell lines Ba/F3 and MCF10A. As shown in FIG. 19, AMOTL2 E507G (RNA edited variant) increased proliferation in both sensor cell lines. The control constructs (for Ba/F3 Mock, pDest GFP and pGIPZ shRNA) had a low background which was similar to AMOTL2 WT (normal AMOTL2 sequence) and AMOTL2 shRNA. In MCF10A, the control constructs (pDEST GFP and shRNA vector) were similar to AMOTL2 WT and AMOTL2 shRNA. Thus, the studies identify AMOTL2 E507G is a driver mutation resulting from elevated editing activity.

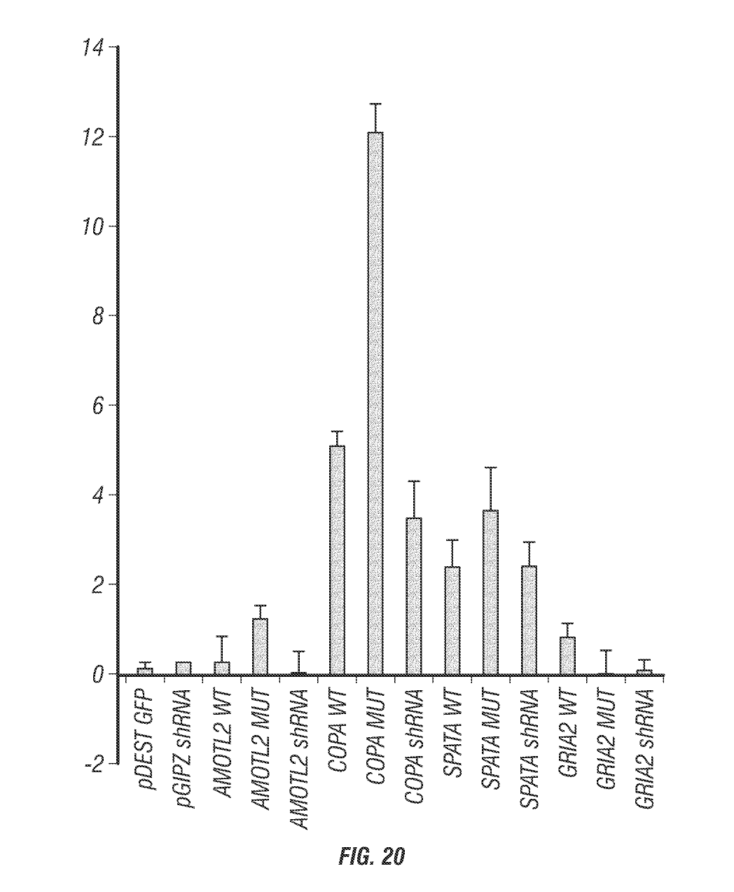

Additional mutations resulting from RNA editing were also studies using the Ba/F3 sensor cells. The FIG. 20 represents a further typical screen of editing events in the Ba/F3 cells. The first two lanes (pDest GFP and pGIPZ shRNA) are controls representing the low signal in the assay. The remaining constructs are in groups of three with the normal wild type (WT) sequence, the RNA edited sequence (MUT) and a shRNA knockdown control. As examples AMOTL2 (AMOTL2 E507G) and COPA (CopA G164V) mutant have a higher level of proliferation than WT or shRNA indicating that these represent driver mutations. The SPATA MUT exhibited only slightly higher activity than WT. In contrast the GRIA2 MUT has a lower proliferation than Wild type and similar to GRIA2 shRNA indicating that the edited variant (MUT) is likely acting as a dominant negative or tumor suppressor and inhibiting cell function. Corresponding studies were undertaken with an edited variant of ANKRD10. In this case, the ANKRD10 D152G edited construct increases the proliferation of the Ba/F3 sensor line and is thus an edited RNA driver.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Bamford, S., Dawson, E., Forbes, S., Clements, J., Pettett, R., Dogan, A., Flanagan, A., Teague, J., Futreal, P. A., Stratton, M. R. et al. 2004. The COSMIC (Catalogue of Somatic Mutations in Cancer) database and website. Br J Cancer 91: 355-358.
Berger, M. F., Levin, J. Z., Vijayendran, K., Sivachenko, A., Adiconis, X., Maguire, J., Johnson, L. A., Robinson, J., Verhaak, R. G., Sougnez, C. et al. 2010. Integrative analysis of the melanoma transcriptome. Genome Res 20: 413-427.
1000 Genome Project Consortium. 2010. A map of human genome variation from population-scale sequencing. Nature 467: 1061-1073.
Sherry, S. T., Ward, M. H., Kholodov, M., Baker, J., Phan, L., Smigielski, E. M., and Sirotkin, K. 2001. dbSNP: the NCBI database of genetic variation. Nucleic Acids Res 29: 308-311.
Wang, K., Li, M., and Hakonarson, H. 2010. ANNOVAR: functional annotation of genetic variants from high-throughput sequencing data. Nucleic Acids Res 38: e164.
Bejsovec A. 2005. Wnt pathway activation: new relations and locations. Cell 120(1): 11-14.
Benjamini Y, Hochberg Y. 1995. Controlling the False Discovery Rate—a Practical and Powerful Approach to Multiple Testing. Journal of the Royal Statistical Society Series B-Methodological 57(1): 289-300.
Byron S A, Gartside M G, Wellens C L, Mallon M A, Keenan J B, Powell M A, Goodfellow P J, Pollock P M. 2008 Inhibition of activated fibroblast growth factor receptor 2 in endometrial cancer cells induces cell death despite PTEN abrogation. Cancer research 68(17): 6902-6907.
Carter H, Samayoa J, Hruban R H, Karchin R. 2010. Prioritization of driver mutations in pancreatic cancer using cancer-specific high-throughput annotation of somatic mutations (CHASM). Cancer Biol Ther 10(6): 582-587.
Catasus L, Gallardo A, Cuatrecasas M, Prat J. 2008. PIK3CA mutations in the kinase domain (exon 20) of uterine endometrial adenocarcinomas are associated with adverse prognostic parameters. Modern Pathol 21(2): 131-139.
2009. Concomitant PI3K-AKT and p53 alterations in endometrial carcinomas are associated with poor prognosis. Modern Pathol 22(4): 522-529.
Chapman M A, Lawrence M S, Keats J J, Cibulskis K, Sougnez C, Schinzel A C, Harview C L, Brunet J P, Ahmann G J, Adli M et al. 2011. Initial genome sequencing and analysis of multiple myeloma. Nature 471(7339): 467-472.
Cheung L W, Hennessy B T, Li J, Yu S, Myers A P, Djordjevic B, Lu Y, Stemke-Hale K, Zhang F, Ju Z et al. 2011. High Frequency of PIK3R1 and PIK3R2 Mutations in Endometrial Cancer Elucidates a Novel Mechanism for Regulation of PTEN Protein Stability. Cancer Discov 1(2): 170-185.
Chin L, Hahn W C, Getz G, Meyerson M. 2011. Making sense of cancer genomic data. Genes Dev 25(6): 534-555.
Cinghu S, Anandharaj A, Lee H C, Yu J R, Park W Y. 2011. FTS (fused toes homolog) a novel oncoprotein involved in uterine cervical carcinogenesis and a potential diagnostic marker for cervical cancer. J Cell Physiol 226(6): 1564-1572.
Davies H, Hunter C, Smith R, Stephens P, Greenman C, Bignell G, Teague J, Butler A, Edkins S, Stevens C et al. 2005. Somatic mutations of the protein kinase gene family in human lung cancer. Cancer research 65(17): 7591-7595.
Dedes K J, Wetterskog D, Ashworth A, Kaye S B, Reis-Filho J S. 2011. Emerging therapeutic targets in endometrial cancer. Nature reviews Clinical oncology 8(5): 261-271.
Di Simone N, Schneyer A L, Caliandro D, Castellani R, Caruso A. 2002. Regulation of endometrial adenocarcinoma cell proliferation by Activin-A and its modulation by 17beta-estradiol. Molecular and cellular endocrinology 192(1-2): 187-195.
Ding L, Ellis M J, Li S, Larson D E, Chen K, Wallis J W, Harris C C, McLellan M D, Fulton R S, Fulton L L et al. 2010. Genome remodelling in a basal-like breast cancer metastasis and xenograft. Nature 464(7291): 999-1005.
Ejskjaer K, Sorensen B S, Poulsen S S, Forman A, Nexo E, Mogensen O. 2007. Expression of the epidermal growth factor system in endometrioid endometrial cancer. Gynecol Oncol 104(1): 158-167.
Ferlay Jea. 2010. GLOBOCAN 2008, cancer incidence and mortality worldwide. in CancerBase. International Agency for Research on Cancer Lyon.
Guan B, Mao T L, Panuganti P K, Kuhn E, Kurman R J, Maeda D, Chen E, Jeng Y M, Wang T L, Shih I M. 2011a. Mutation and Loss of Expression of ARID1A in Uterine Low-grade Endometrioid Carcinoma. Am J Surg Pathol 35(5): 625-632.
Guan B, Wang T L, Shih I M. 2011b. ARID1A, a Factor That Promotes Formation of SWI/SNF-Mediated Chromatin Remodeling, Is a Tumor Suppressor in Gynecologic Cancers. Cancer research 71(21): 6718-6727.
Gui Y, Guo G, Huang Y, Hu X, Tang A, Gao S, Wu R, Chen C, Li X, Zhou L et al. 2011. Frequent mutations of chromatin remodeling genes in transitional cell carcinoma of the bladder. Nat Genet 43(9): 875-878.
Hayashi S, Okada T, Igarashi N, Fujita T, Jahangeer S, Nakamura S. 2002. Identification and characterization of RPK118, a novel sphingosine kinase-1-binding protein. The Journal of biological chemistry 277(36): 33319-33324.
Hayes M P, Douglas W, Ellenson L H. 2009. Molecular alterations of EGFR and PIK3CA in uterine serous carcinoma. Gynecol Oncol 113(3): 370-373.
Hennessy B T, Smith D L, Ram P T, Lu Y, Mills G B. 2005. Exploiting the PI3K/AKT pathway for cancer drug discovery. Nat Rev Drug Discov 4(12): 988-1004.
Hollander M C, Blumenthal G M, Dennis P A. 2011. PTEN loss in the continuum of common cancers, rare syndromes and mouse models. Nat Rev Cancer 11(4): 289-301.
Ikeda T, Yoshinaga K, Semba S, Kondo E, Ohmori H, Horii A. 2000. Mutational analysis of the CTNNB1 (beta-catenin) gene in human endometrial cancer: Frequent mutations at codon 34 that cause nuclear accumulation. Oncol Rep 7(2): 323-326.
Jia L, Liu Y J, Yi X F, Miron A, Crum C P, Kong B, Zheng W X. 2008. Endometrial glandular dysplasia with frequent p53 gene mutation: A genetic evidence supporting its precancer nature for endometrial serous carcinoma. Clin Cancer Res 14(8): 2263-2269.
Jones S, Wang T L, Shih Ie M, Mao T L, Nakayama K, Roden R, Glas R, Slamon D, Diaz L A, Jr., Vogelstein B et al. 2010. Frequent mutations of chromatin remodeling gene ARID1A in ovarian clear cell carcinoma. Science 330(6001): 228-231.
Kaneda H, Arao T, Matsumoto K, De Velasco M A, Tamura D, Aomatsu K, Kudo K, Sakai K, Nagai T, Fujita Y et al. 2011. Activin A inhibits vascular endothelial cell growth and suppresses tumour angiogenesis in gastric cancer. Br J Cancer 105(8): 1210-1217.
Lax S F, Kendall B, Tashiro H, Slebos R J, Hedrick L. 2000. The frequency of p53, K-ras mutations, and microsatellite instability differs in uterine endometrioid and serous carcinoma: evidence of distinct molecular genetic pathways. Cancer 88(4): 814-824.
Liu L L, Yang C Y, Yuan J, Chen X J, Xu J N, Wei Y H, Yang J C, Lin G, Yu L. 2005. RPK118, a PX domain-containing protein, interacts with peroxiredoxin-3 through pseudo-kinase domains. Mol Cells 19(1): 39-45.
Lutz P G, Moog-Lutz C, Coumau-Gatbois E, Kobari L, Di Gioia Y, Cayre Y E. 2000. Myeloblastin is a granulocyte colony-stimulating factor-responsive gene conferring factor-independent growth to hematopoietic cells. P Natl Acad Sci USA 97(4): 1601-1606.
Mamo A, Cavallone L, Tuzmen S, Chabot C, Ferrario C, Hassan S, Edgren H, Kallioniemi O, Aleynikova O, Przybytkowski E et al. 2012. An integrated genomic approach identifies ARID1A as a candidate tumor-suppressor gene in breast cancer. Oncogene 31(16): 2090-2100.
Mardis E R. 2011. A decade's perspective on DNA sequencing technology. Nature 470(7333): 198-203.
Miyake T, Yoshino K, Enomoto T, Takata T, Ugaki H, Kim A, Fujiwara K, Miyatake T, Fujita M, Kimura T. 2008. PIK3CA gene mutations and amplifications in uterine cancers, identified by methods that avoid confounding by PIK3CA pseudogene sequences. Cancer Left 261(1): 120-126.
Moreno-Bueno G, Hardisson D, Sanchez C, Sarrio D, Cassia R, Garcia-Rostan G, Prat J, Guo M, Herman J G, Matias-Guiu X et al. 2002. Abnormalities of the APC/beta-catenin pathway in endometrial cancer. Oncogene 21(52): 7981-7990.
Mosesson Y, Mills G B, Yarden Y. 2008. Derailed endocytosis: an emerging feature of cancer. Nature Reviews Cancer 8(11): 835-850.
Mutter G L, Lin M C, Fitzgerald J T, Kum J B, Baak J P A, Lees J A, Weng L P, Eng C. 2000. Altered PTEN expression as a diagnostic marker for the earliest endometrial precancers. J Natl Cancer I 92(11): 924-931.
Notaridou M, Quaye L, Dafou D, Jones C, Song H, Hogdall E, Kjaer S K, Christensen L, Hogdall C, Blaakaer J et al. 2011. Common alleles in candidate susceptibility genes associated with risk and development of epithelial ovarian cancer. International journal of cancer Journal international du cancer 128(9): 2063-2074.
Oda K, Stokoe D, Taketani Y, McCormick F. 2005. High frequency of coexistent mutations of PIK3CA and PTEN genes in endometrial carcinoma. Cancer research 65(23): 10669-10673.
Park E S, Rabinovsky R, Carey M, Hennessy B T, Agarwal R, Liu W, Ju Z, Deng W, Lu Y, Woo H G et al. 2010. Integrative analysis of proteomic signatures, mutations, and drug responsiveness in the NCI 60 cancer cell line set. Mol Cancer Ther 9(2): 257-267.
Puente X S, Pinyol M, Quesada V, Conde L, Ordonez G R, Villamor N, Escaramis G, Jares P, Bea S, Gonzalez-Diaz M et al. 2011. Whole-genome sequencing identifies recurrent mutations in chronic lymphocytic leukaemia. Nature 475(7354): 101-105.
Remy I, Michnick S W. 2004. Regulation of apoptosis by the Ft1 protein, a new modulator of protein kinase B/Akt. Molecular and cellular biology 24(4): 1493-1504.
Reva B, Antipin Y, Sander C. 2011. Predicting the functional impact of protein mutations: application to cancer genomics. Nucleic Acids Res.
Risbridger G P, Schmitt J F, Robertson D M. 2001. Activins and inhibins in endocrine and other tumors. Endocrine reviews 22(6): 836-858.
Risinger J I, Hayes K, Maxwell G L, Carney M E, Dodge R K, Barrett J C, Berchuck A. 1998. PTEN mutation in endometrial cancers is associated with favorable clinical and pathologic characteristics. Clin Cancer Res 4(12): 3005-3010.
Salvesen H B, Stefansson I, Kretzschmar E I, Gruber P, MacDonald N D, Ryan A, Jacobs I J, Akslen L A, Das S. 2004. Significance of PTEN alterations in endometrial carcinoma: a population-based study of mutations, promoter methylation and PTEN protein expression. International journal of oncology 25(6): 1615-1623.
Schlosshauer P W, Pirog E C, Levine R L, Ellenson L H. 2000. Mutational analysis of the CTNNB1 and APC genes in uterine endometrioid carcinoma. Modern pathology: an official journal of the United States and Canadian Academy of Pathology, Inc 13(10): 1066-1071.
Schramm A, von Schuetz V, Christiansen H, Havers W, Papoutsi M, Wilting J, Schweigerer L. 2005. High activin A-expression in human neuroblastoma: suppression of malignant potential and correlation with favourable clinical outcome. Oncogene 24(4): 680-687.
Seder C W, Hartojo W, Lin L, Silvers A L, Wang Z W, Thomas D G, Giordano T J, Chen G A, Chang A C, Orringer M B et al. 2009. Upregulated INHBA Expression May Promote Cell Proliferation and Is Associated with Poor Survival in Lung Adenocarcinoma. Neoplasia 11(4): 388-396.
Shoji K, Oda K, Nakagawa S, Hosokawa S, Nagae G, Uehara Y, Sone K, Miyamoto Y, Hiraike H, Hiraike-Wada O et al. 2009. The oncogenic mutation in the pleckstrin homology domain of AKT1 in endometrial carcinomas. Br J Cancer 101(1): 145-148.
Sithanandam G, Anderson L M. 2008. The ERBB3 receptor in cancer and cancer gene therapy. Cancer gene therapy 15(7): 413-448.
Srinivasan R, Benton E, McCormick F, Thomas H, Gullick W J. 1999. Expression of the c-erbB-3/HER-3 and c-erbB-4/HER-4 growth factor receptors and their ligands, neuregulin-1 alpha, neuregulin-1 beta, and betacellulin, in normal endometrium and endometrial cancer. Clin Cancer Res 5(10): 2877-2883.
Stefansson I M, Salvesen H B, Akslen L A. 2004. Prognostic impact of alterations in P-cadherin expression and related cell adhesion markers in endometrial cancer. J Clin Oncol 22(7): 1242-1252.
Stephens P, Edkins S, Davies H, Greenman C, Cox C, Hunter C, Bignell G, Teague J, Smith R, Stevens C et al. 2005. A screen of the complete protein kinase gene family identifies diverse patterns of somatic mutations in human breast cancer. Nat Genet 37(6): 590-592.
Sun H, Enomoto T, Fujita M, Wada H, Yoshino K, Ozaki K, Nakamura T, Murata Y. 2001. Mutational analysis of the PTEN gene in endometrial carcinoma and hyperplasia. Am J Clin Pathol 115(1): 32-38.
Tanaka T, Toujima S, Otani T, Minami S, Yamoto M, Umesaki N. 2003. Expression and function of activin receptors in human endometrial adenocarcinoma cells. International journal of oncology 23(3): 657-663.
TCGA Network. 2008. Comprehensive genomic characterization defines human glioblastoma genes and core pathways. Nature 455: 1061-1068.
TCGA Network. 2011. Integrated genomic analyses of ovarian carcinoma. Nature 474: 609-615.
Toyama T, Lee H C, Koga H, Wands J R, Kim M. 2010. Noncanonical Wnt11 inhibits hepatocellular carcinoma cell proliferation and migration. Molecular cancer research: MCR 8(2): 254-265.
Velasco A, Bussaglia E, Pallares J, Dolcet X, Llobet D, Encinas M, Llecha N, Palacios J, Prat J, Matias-Guiu X. 2006. PIK3CA gene mutations in endometrial carcinoma: correlation with PTEN and K-RAS alterations. Human pathology 37(11): 1465-1472.
Wang K, Kan J, Yuen S T, Shi S T, Chu K M, Law S, Chan T L, Kan Z, Chan A S, Tsui W Y et al. 2011. Exome sequencing identifies frequent mutation of ARID1A in molecular subtypes of gastric cancer. Nat Genet.
Warmuth M, Kim S, Gu X J, Xia G, Adrian F. 2007. Ba/F3 cells and their use in kinase drug discovery. Curr Opin Oncol 19(1): 55-60.
Wiegand K C, Lee A F, Al-Agha O M, Chow C, Kalloger S E, Scott D W, Steidl C, Wiseman S M, Gascoyne R D, Gilks B et al. 2011. Loss of BAF250a (ARID1A) is frequent in high-grade endometrial carcinomas. J Pathol 224(3): 328-333.
Wiegand K C, Shah S P, Al-Agha O M, Zhao Y, Tse K, Zeng T, Senz J, McConechy M K, Anglesio M S, Kalloger S E et al. 2010. ARID1A mutations in endometriosis-associated ovarian carcinomas. N Engl J Med 363(16): 1532-1543.
Wu J I, Lessard J, Crabtree G R. 2009. Understanding the Words of Chromatin Regulation. Cell 136(2): 200-206.
Yoda A, Yoda Y, Chiaretti S, Bar-Natan M, Mani K, Rodig S J, West N, Xiao Y, Brown J R, Mitsiades C et al. 2010. Functional screening identifies CRLF2 in precursor B-cell acute lymphoblastic leukemia. P Natl Acad Sci USA 107(1): 252-257.

1-2. (canceled)

3. The method of claim 31, wherein the analyzing of step (iii) comprises:
(A) expressing said plurality of genes, or inhibitory nucleic acid targeted to said plurality of genes, in cells; and
(B) analyzing the effect the expression in the cells to determine the presence of one or more genes having an oncogenic biomarker.

4-5. (canceled)

6. The method of claim 31, wherein selecting one or more candidate agents comprises
screening for an agent effective against cells that express a gene having an oncogenic biomarker or express an inhibitory nucleic acid targeted to a gene having an oncogenic biomarker.

7. The method of claim 31, wherein selecting one or more candidate agents comprises
determining changes in signaling pathway activation in cells that express a gene having an oncogenic biomarker or express an inhibitory nucleic acid targeted to a gene having an oncogenic biomarker; and
selecting one or more candidate agents to treat the patient based on the changes in signaling pathway activation.

8. The method of claim 7, wherein determining changes in signaling pathway activation comprises performing a test using an RNA expression array, RNASeq or reverse-phase protein array.

9. The method of claim 3, wherein analyzing the effect of expression in the cells comprises determining a change in gene expression, cell proliferation, or growth factor dependence upon expression.

10. The method of claim 3, wherein the cells are tissue culture cells.

11. The method of claim 3, wherein the cells are from a patient sample.

12. The method of claim 3, wherein the cells are endometrial cancer cells.

13. The method of claim 4, wherein the tissue culture cells are IL-3 dependent myeloid cells.

14. The method of claim 13, wherein analyzing the effect the expression in the cells comprises determining one or more genes that, upon expression, allow the myeloid cells to proliferate in the absence of IL-3.

15. The method of claim 6, wherein screening for an agent effective against cells that express a gene having an oncogenic biomarker or express an inhibitory nucleic acid targeted to a gene having an oncogenic biomarker comprises determining a change in gene expression, cell proliferation, or growth factor dependence upon expression.

16. The method of claim 31, wherein the candidate agent is an antibody or small molecule.

17. The method of claim 3, wherein the plurality of genes comprising mutations comprise polypeptide coding sequences or miRNA coding sequence.

18. The method of claim 3, wherein the plurality of genes comprising mutations comprise a gene having a nucleotide substitution, deletion or insertion relative to a wild type sequence.

19. The method of claim 31, wherein obtaining genomic sequences of the patient's cancer comprises obtaining expressed RNA or exon sequences of the patient's cancer.

20-23. (canceled)

24. The method of claim 31, wherein the cancer patient has an oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer.

25-28. (canceled)

29. The method of claim 3, wherein step (B) further comprises expressing an inhibitory nucleic acid targeted to said plurality of genes in the tissue culture cells.

30. (canceled)

31. A method of treating a patient having a cancer, the method comprising:
(a) obtaining the results of an analysis comprising:
(i) obtaining genomic sequences of the patient's cancer;
(ii) identifying from said sequence a plurality of genes that are mutated in the patient's cancer; and
(ii) analyzing the plurality of mutant genes to determine the presence of one or more genes having an oncogenic biomarker;
(iv) selecting one or more candidate agents to treat the patient on the basis of said analyzing; and
(b) causing the patient to be treated with the one or more agents so selected.

32-36. (canceled)

37. An in vitro method of selecting a drug to treat a patient having a cancer, the method comprising:
(a) obtaining genomic sequences from DNA or expressed RNA of the patient's cancer;
(b) identifying a plurality of genes that are mutated in the patient's cancer;
(c) expressing said plurality of genes, or inhibitory nucleic acids targeted to said plurality of genes, in cells;
(d) analyzing the effect the expression in the cells to determine the presence of one or more genes having an oncogenic biomarker;
(e) screening for a candidate agent effective against cells that express a gene having an oncogenic biomarker or express an inhibitory nucleic acid targeted to a gene having an oncogenic biomarker;
(f) determining changes in signaling pathway activation in cells that express a gene having an oncogenic biomarker or express an inhibitory nucleic acid targeted to a gene having an oncogenic biomarker; and
(g) selecting one or more candidate agents to treat the patient based on the changes in signaling pathway activation or based on the screening.

38-48. (canceled)

49. A method for treating a cancer patient, wherein it was determined that cancer cells in the patient comprise a mutation in a PIK3R1 that truncates the PIK3R1 open reading frame (ORF), the method comprising administering a MEK or JNK inhibitor therapy to the patient.

50-59. (canceled)

60. A method for treating a cancer patient, wherein it was determined the cancer cells in the patient comprise a KRAS oncogene, the method comprising administering a MEK or p38MAPK inhibitor therapy to the patient.

61-100. (canceled)
